(19) United States
(12) Patent Application Publication
(10) Pub. No.: US 2024/0091226 A1
(43) Pub. Date: Mar. 21, 2024

(54) Forms and Formulations Of A Tyrosine Kinase Non-Receptor 1 (TNK1) Inhibitor

(71) Applicant: Sumitomo Pharma Oncology, inc., Marlborough, MA (US)

(72) Inventors: J. Micah Wilcox, Layton, UT (US); Jason Marc Foulks, Sandy, UT (US); Steven L. Warner, Draper, UT (US)

(21) Appl. No.: 18/260,170

(22) PCT Filed: Jan. 4, 2022
(86) PCT No.: PCT/US2022/070007

§ 371 Date: Jun. 30, 2023

Related U.S. Application Data
(60) Provisional application No. 63/133,983, filed on Jan. 5, 2021.

(51) Int. Cl. A61K 31/506; A61K 9/48; A61K 45/06; A61P 35/00; C07D 401/14

(52) U.S. Cl. CPC A61K 31/506; A61K 9/4858; A61K 9/4866; A61K 45/06; A61P 35/00; C07D 401/14; C07B 2200/13

(57) ABSTRACT

Provided herein are compositions of matter, e.g. solid forms, pharmaceutical compositions, pharmaceutical combinations and unit dosage forms, of a compound of the following structural formula: (I) or a pharmaceutically acceptable salt thereof, or a hydrate of either of the foregoing. The compositions of matter described herein can be used to treat tyrosine kinase non-receptor 1 (TNK1)-mediated diseases, disorders and/or conditions.

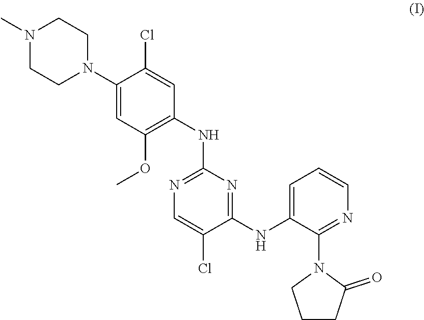 [SMILES: COc1cc(N2CCN(C)CC2)c(Cl)cc1Nc1ncc(Cl)c(Nc2cccnc2N2CCCC2=O)n1]

### RELATED APPLICATION

This application claims the benefit of U.S. Provisional Application No. 63/133,983, filed on Jan. 5, 2021. The entire teachings of this application are incorporated herein by reference.

### BACKGROUND

Tyrosine kinase non-receptor 1 (TNK1) is a member of the ACK family of non-receptor tyrosine kinases, and its dysregulation has been linked to disorders such as cancer.

Accordingly, there is a need for new treatments and therapies for TNK1-mediated diseases, disorders and conditions.

### SUMMARY

Provided herein are compositions of matter, e.g., solid forms, such as crystalline and polymorphic forms, pharmaceutical compositions, pharmaceutical combinations and unit dosage forms, of a compound that inhibits TNK1. The compositions of matter described herein can be used in methods of treating TNK1-mediated diseases, disorders and/or conditions (e.g., disorders or diseases), e.g., as described herein.

One aspect is a solid form (e.g., crystalline form, polymorphic form) of a mesylate salt of a compound of the following structural formula:

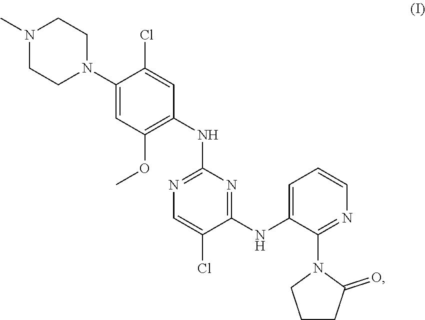 [SMILES: COc1cc(N2CCN(C)CC2)c(Cl)cc1Nc1ncc(Cl)c(Nc2cccnc2N2CCCC2=O)n1]

or a hydrate thereof.

Another aspect is a pharmaceutical composition comprising a solid form (e.g., crystalline form, polymorphic form) described herein and a pharmaceutically acceptable carrier.

Another aspect is a pharmaceutical combination comprising a solid form (e.g., crystalline form, polymorphic form) described herein and one or more additional therapeutic agents.

Yet another aspect is a pharmaceutical composition comprising a compound of structural formula I, or a pharmaceutically acceptable salt thereof, or a hydrate of the foregoing; and silicified microcrystalline cellulose, croscarmellose sodium or sodium stearyl fumarate.

Another aspect is a unit dosage form comprising a pharmaceutical composition described herein.

Another aspect is a method of treating a disease, disorder or condition described herein (e.g., a TNK1-mediated disease, disorder or condition; cancer; inflammatory disorder; tissue injury; a disease, disorder or condition that would benefit from improved intestinal barrier function; splenomegaly) in a subject, such as a subject in need thereof, comprising administering to the subject (e.g., a therapeutically effective amount of) a solid form (e.g., crystalline form, polymorphic form), pharmaceutical composition, pharmaceutical combination or unit dosage form described herein. Another aspect is a solid form (e.g., crystalline form, polymorphic form), pharmaceutical composition, pharmaceutical combination or unit dosage form described herein for use in treating a disease, disorder or condition described herein (e.g., a TNK1-mediated disease, disorder or condition; cancer; inflammatory disorder; tissue injury; a disease, disorder or condition that would benefit from improved intestinal barrier function; splenomegaly), e.g., in a subject, such as a subject in need thereof. Another aspect is use of a solid form (e.g., crystalline form, polymorphic form), pharmaceutical composition, pharmaceutical combination or unit dosage form described herein for the manufacture of a medicament for treating a disease, disorder or condition described herein (e.g., a TNK1-mediated disease, disorder or condition; cancer; inflammatory disorder; tissue injury; a disease, disorder or condition that would benefit from improved intestinal barrier function; splenomegaly), e.g., in a subject, such as a subject in need thereof.

Another aspect is a method of treating a TNK1-mediated disease, disorder or condition in a subject carrying a TNK1 mutation, comprising determining whether the subject carries a TNK1 mutation; and administering to the subject (e.g., a therapeutically effective amount of) a solid form (e.g., crystalline form, polymorphic form), pharmaceutical composition, pharmaceutical combination or unit dosage form described herein if it is determined that the subject carries the TNK1 mutation.

Another aspect is a method of reducing inflammation in a subject, such as a subject in need thereof, comprising administering to the subject (e.g., a therapeutically effective amount of) a solid form (e.g., crystalline form, polymorphic form), pharmaceutical composition, pharmaceutical combination or unit dosage form described herein. Another aspect is a solid form (e.g., crystalline form, polymorphic form), pharmaceutical composition, pharmaceutical combination or unit dosage form described herein for use in reducing inflammation, e.g., in a subject, such as a subject in need thereof. Another aspect is use of a solid form (e.g., crystalline form, polymorphic form), pharmaceutical composition, pharmaceutical combination or unit dosage form described herein for the manufacture of a medicament for reducing inflammation, e.g., in a subject, such as a subject in need thereof.

Another aspect is a method of improving intestinal barrier function in a subject, such as a subject in need thereof, comprising administering to the subject (e.g., a therapeutically effective amount of) a solid form (e.g., crystalline form, polymorphic form), pharmaceutical composition, pharmaceutical combination or unit dosage form described herein. Another aspect is a solid form (e.g., crystalline form, polymorphic form), pharmaceutical composition, pharmaceutical combination or unit dosage form described herein for use in improving intestinal barrier function, e.g., in a subject, such as a subject in need thereof. Another aspect is use of a solid form (e.g., crystalline form, polymorphic form), pharmaceutical composition, pharmaceutical combination or unit dosage form described herein for the manufacture of a medicament for improving intestinal barrier function, e.g., in a subject, such as a subject in need thereof.

Another aspect is a method of mediating apoptosis and/or reducing inflammation and/or inhibiting TNK1 activity in a cell, comprising contacting the cell with a solid form (e.g., crystalline form, polymorphic form), pharmaceutical composition, pharmaceutical combination or unit dosage form described herein.

Yet another aspect is a method of inhibiting TNK1 activity in a subject, such as a subject in need thereof, comprising administering to the subject (e.g., a therapeutically effective amount of) a solid form (e.g., crystalline form, polymorphic form), pharmaceutical composition, pharmaceutical combination or unit dosage form described herein. Another aspect is a solid form (e.g., crystalline form, polymorphic form), pharmaceutical composition, pharmaceutical combination or unit dosage form described herein for use in inhibiting TNK1 activity, e.g., in a subject, such as a subject in need thereof. Another aspect is use of a solid form (e.g., crystalline form, polymorphic form), pharmaceutical composition, pharmaceutical combination or unit dosage form described herein for the manufacture of a medicament for inhibiting TNK1 activity, e.g., in a subject, such as a subject in need thereof.

Another aspect is a method of making a mesylate salt of a compound of structural formula I, or a hydrate thereof, comprising contacting the compound of structural formula I with methanesulfonic acid in a solvent, thereby making the mesylate salt of a compound of structural formula I, or a hydrate thereof.

### BRIEF DESCRIPTION OF THE DRAWINGS

The foregoing will be apparent from the following more particular description of example embodiments.

FIG. 1 is an x-ray powder diffraction (XRPD) spectrum of Mesylate Type A stored for six months at 25° C. and 60% relative humidity in a closed container.

FIG. 2 shows thermogravimetric analysis (TGA) and differential scanning calorimetry (DSC) curves of Mesylate Type A (820105-01-D7) prepared in accordance with the procedure described in Table 1.4.

FIG. 3 is an overlay of XRPD spectra of L-Malate Type B batches.

FIG. 4 is an overlay of XRPD spectra of Succinate Type B batches.

FIG. 5 is an overlay of VT-XRPD spectra of Mesylate Type A subject to the indicated conditions.

FIG. 6 is an overlay of XRPD spectra of freebase forms of the compound of structural formula I.

FIG. 7 is an XRPD spectrum of Mesylate Type A stored for six months at 40° C. and 75% relative humidity in a closed container.

FIG. 8A shows body weight growth curves for female Apc^{Min+/-} mice in the ApcMin mouse model of Example 6.

FIG. 8B shows compound of structural formula I inhibits polyp growth in the ApcMin mouse model of Example 6.

FIG. 8C shows splenomegaly and inflammation is attenuated in mice treated with compound of structural formula I in the ApcMin mouse model of Example 6.

### DETAILED DESCRIPTION

A description of example embodiments follows.

### Definitions

For purposes of interpreting this specification, the following definitions will apply, and whenever appropriate, terms used in the singular will also include the plural. Terms used in the specification have the following meanings unless the context clearly indicates otherwise.

The steps of all methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the present disclosure otherwise claimed.

The terms “a,” “an,” “the” and similar terms used in the context of the present disclosure (especially in the context of the claims) are to be construed to cover both the singular and plural unless otherwise indicated herein or clearly contradicted by the context.

The term “about” means within an acceptable error range for the particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of ±20%, ±15%, ±10%, ±5%, ±4%, 3%, ±2% or 1% of a given value. In some embodiments, “about” is ±20%, e.g., ±15%, 10%, ±5%, 4%, ±3%, ±2% or 1%, of a given value. It is to be understood that the term “about” can precede any particular value specified herein, except for particular values used in the Figures and Examples herein.

As a person of ordinary skill in the art would understand, for example, a ketone (—C(H)C(O)) group in a molecule may tautomerize to its enol form (—C═C(OH)). This disclosure is intended to cover all possible tautomers even when a structure depicts only one of them.

The phrase “pharmaceutically acceptable” means that the substance or composition the phrase modifies must be, within the scope of sound medical judgment, suitable for use in contact with the tissues of humans and lower animals without undue toxicity, irritation, allergic response and the like, and are commensurate with a reasonable benefit/risk ratio. If a substance is part of a composition or formulation, the substance must also be compatible chemically and/or toxicologically with the other ingredients in the composition or formulation.

Unless specified otherwise, the term “compounds of the present disclosure” refers to a compound of any structural formula depicted herein (e.g., a compound of structural formula I), or a salt thereof, as well as isomers, such as stereoisomers (including diastereoisomers, enantiomers and racemates), geometrical isomers, conformational isomers (including rotamers and astropisomers) and tautomers, isotopically labeled compounds (including deuterium substitutions), and inherently formed moieties (e.g., polymorphs and/or solvates, such as hydrates) of the foregoing.

Pharmaceutically acceptable salts are preferred. However, other salts may be useful, e.g., in isolation or purification steps which may be employed during preparation, and thus, are contemplated to be within the scope of the present disclosure.

As used herein, “pharmaceutically acceptable salts” refers to salts derived from suitable inorganic and organic acids and bases that are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable acid addition salts include, but are not limited to, acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethanedisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate and xinafoate salts.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, or copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Examples of organic amines include, but are not limited to, isopropylamine, benzathine, cholinate, diethanolamine, diethylamine, lysine, meglumine, piperazine and tromethamine.

The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Allen, L. V., Jr., ed., Remington: The Science and Practice of Pharmacy, 22nd Edition, Pharmaceutical Press, London, UK (2012), the relevant disclosure of which is hereby incorporated by reference in its entirety.

When a compound of the present disclosure has an asymmetric center, chiral axis, and/or chiral plane (e.g., as described in: E. L. Eliel and S. H. Wilen, Stereo-chemistry of Carbon Compounds, John Wiley & Sons, New York, 1994, pages 1119-1190), such compound may occur as a racemic mixture, individual isomer (e.g., diastereomer, enantiomer, geometrical isomer, conformational isomer (including rotamers and atropisomers), tautomer) and/or intermediate mixture, with all possible isomers and mixtures thereof being included herein.

As used herein, the term “isomers” refers to different compounds that have the same molecular formula but differ in arrangement and configuration of the atoms.

“Enantiomers” are a pair of stereoisomers that are non-superimposable mirror images of each other. A 1:1 mixture of a pair of enantiomers is a “racemic” mixture. “Racemate” or “racemic” is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g., (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Geometric isomers may occur when a compound contains a double bond or some other feature that gives the molecule a certain amount of structural rigidity. If the compound contains a double bond, the double bond may be E- or Z-configuration. If the compound contains a disubstituted cycloalkyl, the cycloalkyl substituent may have a cis- or trans-configuration.

Conformational isomers (or conformers) are isomers that can differ by rotations about one or more bonds. Rotamers are conformers that differ by rotation about only a single bond.

The term “atropisomer,” as used herein, refers to a structural isomer based on axial or planar chirality resulting from restricted rotation in the molecule.

Optically active (R)- and (S)-isomers may be prepared using chiral synthons or chiral reagents, or resolved using conventional techniques (e.g., separated on chiral SFC or HPLC chromatography columns, such as CHIRALPAK® and CHIRALCEL® columns available from DAICEL Corp. or other equivalent columns, using the appropriate solvent or mixture of solvents to achieve suitable separation).

Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization.

Depending on the process conditions, the end products of the present disclosure are obtained either in free (neutral) or salt form. Both the free form and the salts of these end products are within the scope of the present disclosure. If so desired, one form of a compound may be converted into another form. A free base or acid may be converted into a salt; a salt may be converted into the free compound or another salt; a mixture of isomeric compounds of the present disclosure may be separated into the individual isomers.

Compounds of the present disclosure that contain groups capable of acting as donors and/or acceptors for hydrogen bonds may be capable of forming co-crystals with suitable co-crystal formers. These co-crystals may be prepared from compounds of the present disclosure by known co-crystal forming procedures. Such procedures include grinding, heating, co-subliming, co-melting, or contacting in solution compounds of the present disclosure with the co-crystal former under crystallization conditions and isolating co-crystals thereby formed. Suitable co-crystal formers include those described in WO 2004/078163. Hence, the present disclosure further provides co-crystals comprising a compound of the present disclosure and a co-crystal former.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorous, fluorine, chlorine and iodine, such as ²H, ³H, ¹¹C, ¹³C, ¹⁴C, ¹⁵N ¹⁸F, ³¹P, ³²P, ³⁵S, ³⁶Cl, ¹²³I, ¹²⁴I and ¹²⁵I, respectively. The present disclosure includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound of the present disclosure. The concentration of such a heavier isotope, specifically deuterium, may be defined by the isotopic enrichment factor. The term “isotopic enrichment factor,” as used herein, means the ratio between the isotopic abundance and the natural abundance of a specified isotope. If a substituent in a compound of this present disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically labeled compounds of the present disclosure can generally be prepared by conventional techniques known to those skilled in the art or by processes disclosed in the schemes or in the examples and preparations described below (or analogous processes to those described hereinbelow), by substituting an appropriate or readily available isotopically labeled reagent for a non-isotopically labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

The term “solvate” means a physical association of a compound of the present disclosure with one or more solvent molecules, whether organic or inorganic. This physical association includes hydrogen bonding. In certain instances, the solvate will be capable of isolation, for example, when one or more solvent molecules are incorporated in the crystal lattice of a crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution phase and isolable solvates. Examples of solvates include, but are not limited to, hydrates (a solvate wherein the solvent molecule is water), ethanolates, methanolates, and isopropanolates. Methods of solvation are generally known in the art.

Compounds of the present disclosure can be provided in solid form, e.g., as amorphous solids or crystalline solids. Lyophilization, for example, can be employed to provide the compounds of the present disclosure in solid form.

“Crystalline,” as used herein, refers to a homogeneous solid formed by a repeating, three-dimensional pattern of atoms, ions or molecules having fixed distances between constituent parts. The unit cell is the simplest repeating unit in this pattern. Notwithstanding the homogenous nature of an ideal crystal, a perfect crystal rarely, if ever, exists. “Crystalline,” as used herein, encompasses crystalline forms that include crystalline defects, for example, crystalline defects commonly formed by manipulating (e.g., preparing, purifying) the crystalline forms described herein. A person skilled in the art is capable of determining whether a sample of a compound is crystalline notwithstanding the presence of such defects.

As used herein, “polymorph(s)” or “polymorphic form(s)” refers to crystalline form(s) having the same chemical structure/composition but different spatial arrangements of the molecules and/or ions forming the crystals. Polymorphs can be characterized by analytical methods such as x-ray powder diffraction (XRPD), differential scanning calorimetry (DSC) and thermogravimetric analysis.

The solid forms (e.g., crystalline forms and/or polymorphs) described herein can be substantially pure. As used herein, “substantially pure,” used without further qualification, means the indicated compound has a purity greater than 90 weight percent, for example, greater than 90, 91, 92, 93, 94, 95, 96, 97, 98, or 99 weight percent, and also including a purity equal to about 100 weight percent, based on the weight of the compound. The remaining material comprises, e.g., other form(s) of the compound, and/or reaction impurities and/or processing impurities arising from its preparation. Purity can be assessed using techniques known in the art, for example, using an HPLC assay described herein. “Substantially pure” can also be qualified as in “substantially pure of other physical forms of the compound of structural formula I, or a pharmaceutically acceptable salt thereof, or a hydrate of the foregoing.” When qualified thus, “substantially pure” means that the indicated compound contains less than 10%, preferably less than 5%, more preferably less than 3%, most preferably, less than 1% by weight of the indicated impurity.

An XRPD pattern or DSC thermogram that is “substantially in accordance” with one or more figures herein showing an XRPD pattern or diffractogram or DSC thermogram, respectively, is one that would be considered by one skilled in the art to represent the same single crystalline form of the compound of structural formula I, or a pharmaceutically acceptable salt thereof, or a hydrate of the foregoing, as the sample of the compound of the compound of structural formula I, or a pharmaceutically acceptable salt thereof, or a hydrate of the foregoing, that provided the pattern or diffractogram or thermogram of one or more figures provided herein. Thus, an XRPD pattern or DSC thermogram that is substantially in accordance may be identical to that of one of the figures or, more likely, may be somewhat different from one or more of the figures. For example, an XRPD pattern that is somewhat different from one or more of the figures may not necessarily show each of the lines of the diffraction pattern presented herein and/or may show a slight change in appearance or intensity of the lines or a shift in the position of the lines. These differences typically result from differences in the conditions involved in obtaining the data or differences in the purity of the sample used to obtain the data. A person skilled in the art is capable of determining if a sample of a crystalline compound is of the same form as or a different form from a form disclosed herein by comparison of the XRPD pattern or DSC thermogram of the sample and the corresponding XRPD pattern or DSC thermogram disclosed herein.

It is to be understood that, unless otherwise indicated, any 2θ angle specified herein, with the exception of the 2θ angles in the Figures or Examples, means the specified value±0.2°. For example, when an embodiment or a claim specifies a 2θ angle of 7.5°, without more, this is to be understood to mean 7.5° 0.2°, that is a 2θ angle of from 7.3° to 7.7°. In preferred embodiments, a 2θ angle is the specified value±0.10, in more preferred embodiments, the specified value±0.05°.

The crystalline forms provided herein can also be identified on the basis of differential scanning calorimetry (DSC) and/or thermogravimetric analysis (TGA). DSC is a thermoanalytical technique in which the difference in the amount of heat required to increase the temperature of a sample is measured as a function of temperature. DSC can be used to detect physical transformations, such as phase transitions, of a sample. For example, DSC can be used to detect the temperature(s) at which a sample undergoes crystallization, melting or glass transition. It is to be understood that any temperature associated with DSC specified herein, with the exception of the DSC temperatures in the Figures or Examples, means the specified value ±5° C. or less. For example, when an embodiment or a claim specifies an endothermic peak at 264° C., this is to be understood to mean 264° C.±5° C. or less, that is a temperature of from 259° C. to 269° C. In preferred embodiments, a DSC is the specified value±3° C. or less, in more preferred embodiments, +2° C. or less.

The terms “tyrosine kinase non-receptor inhibitor 1-mediated disease, disorder or condition” and “TNK1-mediated disease, disorder or condition,” as used herein, refer to any disease, disorder or condition which is directly or indirectly regulated by TNK1. Non-limiting examples of a TNK1-mediated disease, disorder or condition include cancer, a gastrointestinal disorder, an inflammatory disorder, tissue injury, multi-organ dysfunction syndrome (MODS), sepsis, an autoimmune disorder, a disease, disorder or condition of the microbiome or a disease, disorder or condition resulting from a trauma and/or intestinal injury.

The terms “malignancy” and “cancer” are used interchangeably herein, and refer to diseases in which abnormal cells divide without control and can invade nearby tissues. Malignant cells can also spread to other parts of the body through the blood and lymph systems. There are several main types of malignancy. Carcinoma is a malignancy that begins in the skin or in tissues that line or cover internal organs. Sarcoma is a malignancy that begins in bone, cartilage, fat, muscle, blood vessels, or other connective or supportive tissue. Leukemia is a malignancy that starts in blood-forming tissue, such as the bone marrow, and causes large numbers of abnormal blood cells to be produced and enter the blood. Lymphoma and multiple myeloma are malignancies that begin in the cells of the immune system. Central nervous system cancers are malignancies that begin in the tissues of the brain and spinal cord.

The term “solid tumor,” as used herein, refers to malignancies/cancers formed of abnormal masses of tissue that usually do not contain cysts or liquid areas. Solid tumors are named/classified according to the tissue/cells of origin. Examples include, but are not limited to, sarcomas and carcinomas.

The term “leukemia,” as used herein, refers to hematologic or blood cell malignancies/cancers that begin in blood-forming tissue, such as the bone marrow. Examples include, but are not limited to, chronic leukemia, acute leukemia, acute myeloid leukemia (AML), chronic myeloid leukemia (CML), acute lymphocytic leukemia (ALL), acute lymphoblastic leukemia (e.g., B-cell, T-cell) and chronic lymphocytic leukemia (CLL).

The term “lymphoma,” as used herein, refers to lymphatic cell malignancies/cancers that begin in the cells of the immune system. Examples include, but are not limited to, Hodgkin's lymphoma, non-Hodgkin's lymphoma and multiple myeloma.

As used herein, the term “subject” refers to an animal. Typically, the animal is a mammal. A subject also refers to, for example, primates (e.g., humans), cows, sheep, goats, horses, dogs, cats, rabbits, rats, mice, fish, birds and the like. In certain embodiments, the subject is a primate. In yet other embodiments, the subject is a human.

As used herein, a subject (e.g., a human) is “in need of” a treatment if such subject would benefit biologically, medically or in quality of life from such treatment.

“Treat,” “treating” and “treatment,” as used herein, refer to the administration of a medication or medical care to a subject, such as a human, having a disease or condition of interest, e.g., a cancer, and includes: (i) preventing the disease or condition from occurring in a subject, in particular, when such subject is predisposed to the condition but has not yet been diagnosed as having it; (ii) inhibiting the disease or condition, e.g., arresting its development; (iii) relieving the disease or condition, e.g., causing regression of the disease or condition; or (iv) relieving the symptoms resulting from the disease or condition (e.g., pain, weight loss, cough, fatigue, weakness, etc.).

The term “a therapeutically effective amount,” as used herein, refers to an amount of a therapeutic agent, such as a compound of the present disclosure, that, when administered to a subject, such as a human, is sufficient to effect treatment. The amount of a therapeutic agent that constitutes an “effective amount” will vary depending on the therapeutic agent, the condition being treated and its severity, the manner of administration, the duration of treatment, or the subject to be treated (e.g., age, weight, fitness of the subject), but can be determined routinely by one of ordinary skill in the art based on his own knowledge and this disclosure. In embodiments, an “effective amount” effects treatment as measured by a statistically significant change in one or more indications, symptoms, signs, diagnostic tests, vital signs, and the like. In other embodiments, an “effective amount” manages or prevents a condition as measured by a lack of a statistically significant change in one or more indications, symptoms, signs, diagnostic tests, vital signs, and the like.

The regimen of administration can affect what constitutes a therapeutically effective amount. A compound of the present disclosure can be administered to the subject either prior to or after the onset of a TNK1-mediated condition. Further, several divided dosages, as well as staggered dosages, can be administered daily or sequentially, or the dose can be continuously infused, or can be a bolus injection. Further, the dosages of the compound(s) of the present disclosure can be proportionally increased or decreased as indicated by the exigencies of the therapeutic or prophylactic situation.

### Solid Forms

It has been found that the compound of structural formula I, or a pharmaceutically acceptable salt thereof, or a hydrate of the foregoing, can exist in various solid, crystalline and polymorphic forms.

In one aspect, provided herein is a mesylate salt of a compound of the following structural formula:

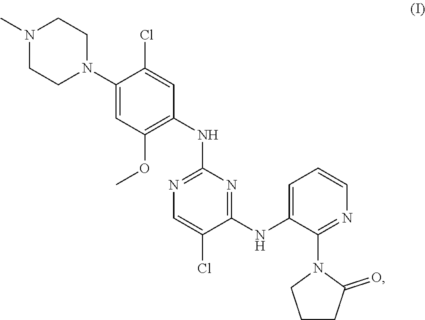 [SMILES: COc1cc(N2CCN(C)CC2)c(Cl)cc1Nc1ncc(Cl)c(Nc2cccnc2N2CCCC2=O)n1]

or a hydrate thereof (e.g., a mesylate salt of a compound of structural formula I). In some aspects, the mesylate salt is in solid form. In some aspects, the mesylate salt is in crystalline form. In some aspects, a solid and/or crystalline form of a mesylate salt of a compound of structural formula I, or a hydrate thereof (e.g., a mesylate salt of a compound of structural formula I), comprises Type A. In some aspects, a solid and/or crystalline form of a mesylate salt of a compound of structural formula I, or a hydrate thereof (e.g., a mesylate salt of a compound of structural formula I), consists essentially of Type A. In some aspects, a solid and/or crystalline form of a mesylate salt of a compound of structural formula I, or a hydrate thereof (e.g., a mesylate salt of a compound of structural formula I), consists of Type A. In a further aspect of any of the foregoing aspects, the mesylate salt is substantially pure.

Type A corresponds to a mesylate salt of the compound of structural formula I and is characterized, in some aspects, by an XRPD pattern comprising at least three peaks (e.g., three peaks, at least four peaks, four peaks or five peaks) at 2-theta angles selected from the group consisting of 7.5±0.2°, 8.8±0.2°, 18.7±0.2°, 20.2±0.2° and 25.2±0.2°. In some aspects, Type A is characterized by an XRPD pattern comprising peaks at the following 2-theta angles: 7.5±0.2°, 8.8±0.2° and 18.7±0.2°. In some aspects, Type A is characterized by an XRPD pattern comprising peaks at the following 2-theta angles: 7.5±0.2°, 8.8±0.2°, 18.7±0.2° and 20.2±0.2°. In further aspects of any of the foregoing aspects, Type A is characterized by an x-ray powder diffraction pattern further comprising a peak at the following 2-theta angle: 16.9±0.2°. In further aspects of any of the foregoing aspects, Type A is characterized by an x-ray powder diffraction pattern further comprising a peak at the following 2-theta angle: 12.4±0.2°. In further aspects of any of the foregoing aspects, Type A is characterized by an x-ray powder diffraction pattern further comprising a peak at the following 2-theta angle: 20.6±0.2°. In further aspects of any of the foregoing aspects, Type A is characterized by an x-ray powder diffraction pattern further comprising a peak at the following 2-theta angle: 15.2±0.2°. In further aspects of any of the foregoing aspects, Type A is characterized by an XRPD pattern further comprising a peak at any one or more of the following 2-theta angles: 4.4±0.2°, 10.7±0.2°, 14.5±0.2°, 21.9±0.2° and 24.5±0.2°.

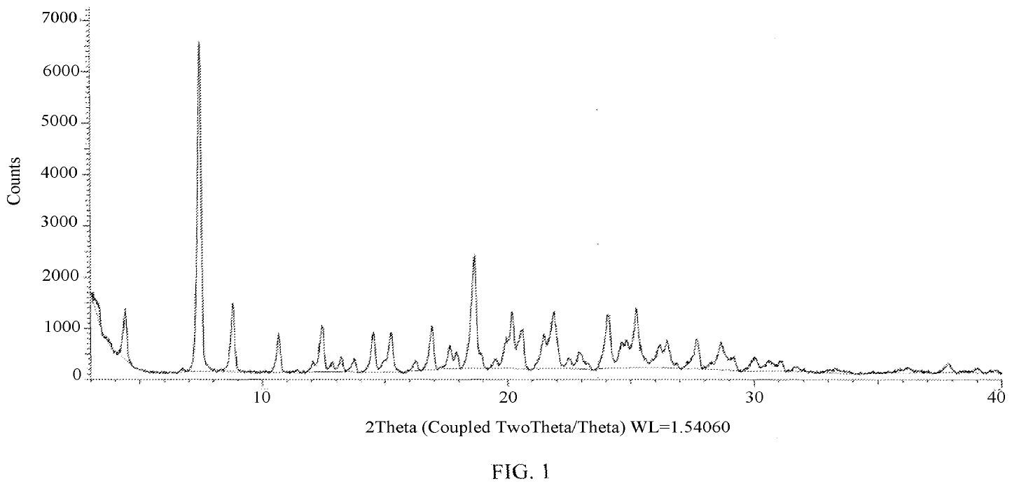

In some aspects, Type A has an XRPD pattern substantially in accordance with that depicted in FIG. 1. In some aspects, the XRPD pattern of Type A is substantially in accordance with that depicted in FIG. 1 after storage for six months at about 25° C. and about 60% relative humidity in a closed container.

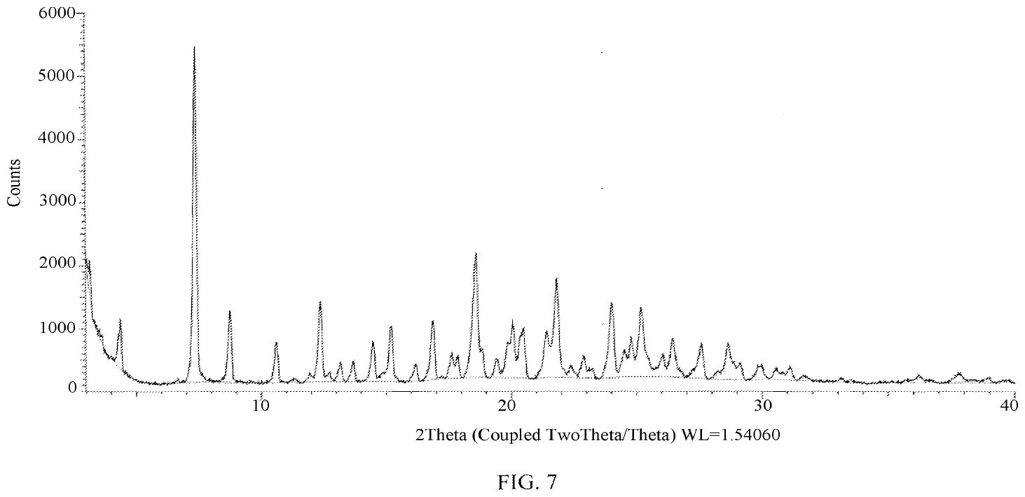

In some aspects, Type A has an XRPD pattern substantially in accordance with that depicted in FIG. 7. In some aspects, the XRPD pattern of Type A is substantially in accordance with that depicted in FIG. 7 after storage for six months at about 40° C. and about 75% relative humidity in a closed container.

In any of the foregoing aspects, the XRPD pattern is as measured by XRPD using an x-ray wavelength of 1.5406 Å.

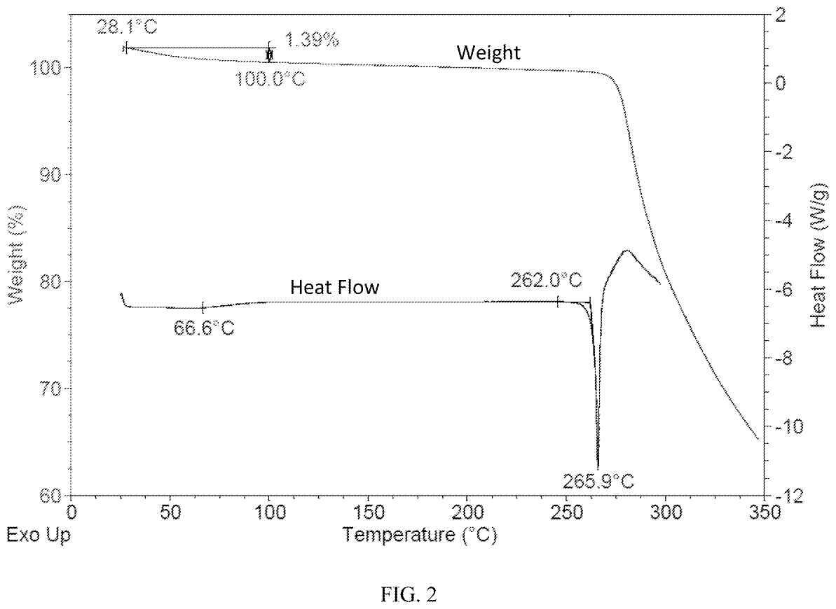

In some aspects, Type A is characterized by a DSC thermogram comprising an endothermic peak at 266° C. In some aspects, Type A is characterized or further characterized by a DSC thermogram comprising a thermal signal at 67° C. In some aspects, Type A has a DSC thermogram substantially in accordance with that depicted in FIG. 2. In any of the foregoing aspects involving DSC, the DSC thermogram is as measured by differential scanning calorimetry over a range of 25° C. to 300° C. using a scanning rate of 10° C./minute.

In some aspects, Type A is characterized by a melting temperature of 262° C., e.g., as measured by DSC.

In some aspects, Type A is characterized by a TGA thermal curve with about 1.4% weight loss over the range of from about 25° C. to about 100° C. In some aspects, Type A has a TGA thermal curve substantially in accordance with that shown in FIG. 2. In any of the foregoing aspects, the TGA thermal curve is as measured using a heating rate of 10° C./minute.

A method of making a compound of structural formula I, or a pharmaceutically acceptable salt thereof, or a hydrate of the foregoing, is disclosed in International Appln. No. PCT/US2020/040737, the entire content of which is incorporated herein by reference, and described in Example 5 herein. Methods of transforming the free base of a compound of structural formula I into a pharmaceutically acceptable salt thereof, or hydrate of the foregoing, are described in Example 1 herein.

Also provided herein is a method of making a mesylate salt of a compound of structural formula I, or a hydrate thereof (e.g., a mesylate salt of a compound of structural formula I). The method comprises contacting the compound of structural formula I with methanesulfonic acid in a solvent (e.g., an organic solvent, or an aqueous mixture thereof), thereby making the mesylate salt of a compound of structural formula I, or a hydrate thereof. In some aspects, the method comprises forming a mixture (e.g., a solution, a suspension) of the compound of structural formula I in a solvent (e.g., an organic solvent, or an aqueous mixture thereof), and contacting the mixture with methanesulfonic acid, thereby making the mesylate salt of a compound of structural formula I, or a hydrate thereof. In some aspects, the method further comprises filtering the mixture to form a filtrate, and contacting the filtrate with methanesulfonic acid.

In some aspects, e.g., to make a solid and/or crystalline form of the mesylate salt of a compound of structural formula I, such as Type A, the method further comprises coupling a compound of the following structural formula:

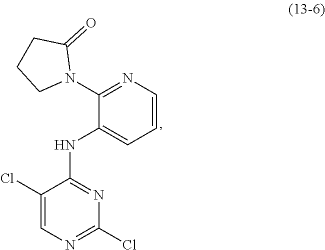 [SMILES: O=C1CCCN1c1ncccc1Nc1nc(Cl)ncc1Cl]

or a salt thereof, to a compound of the following structural formula:

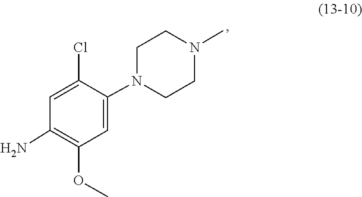 [SMILES: COc1cc(N2CCN(C)CC2)c(Cl)cc1N]

or a salt thereof (e.g., in the presence of a solvent, such as a polar protic solvent, such as tert-butanol, and an acid, such as trifluoroacetic acid), thereby making the compound of structural formula I, or a salt thereof (e.g., the compound of structural formula I). In some further aspects, the method further comprises coupling a compound of the following structural formula:

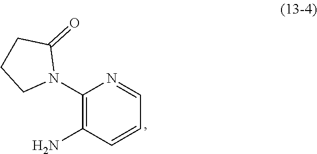 [SMILES: Nc1cccnc1N1CCCC1=O]

or a salt thereof, to a compound of the following structural formula:

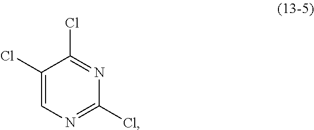

or a salt thereof (e.g., in the presence of a solvent, such as a polar aprotic solvent, such as dimethylformamide, and a base, such as an amine base, such as diisopropylethylamine), thereby making the compound of structural formula 13-6, or a salt thereof.

In a particular aspect, the method of making a mesylate salt of a compound of structural formula I, or a hydrate thereof (e.g., a mesylate salt of a compound of structural formula I), comprises coupling a compound of structural formula (13-4), or a salt thereof, to a compound of structural formula (13-5), or a salt thereof (e.g., in the presence of a solvent, such as a polar aprotic solvent, such as dimethylformamide, and a base, such as an amine base, such as diisopropylethylamine), thereby making a compound of structural formula 13-6, or a salt thereof, coupling the compound of structural formula (13-6), or a salt thereof, to a compound of structural formula (13-10), or a salt thereof (e.g., in the presence of a solvent, such as a polar protic solvent, such as tert-butanol, and an acid, such as trifluoroacetic acid), thereby making a compound of structural formula I; and forming a mixture (e.g., a solution, a suspension) of the compound of structural formula I in a solvent (e.g., an organic solvent, or an aqueous mixture thereof), and contacting the mixture with methanesulfonic acid, thereby making the mesylate salt of a compound of structural formula I, or a hydrate thereof. In some aspects, the method further comprises filtering the mixture to form a filtrate, and contacting the filtrate with methanesulfonic acid.

In some aspects, e.g., to make a solid and/or crystalline form of the mesylate salt of a compound of structural formula I, such as Type A, the method further comprises precipitating the mesylate salt of a compound of structural formula I, or a hydrate thereof (e.g., from a solvent or mixture). In some aspects, the precipitated mesylate salt of a compound of structural formula I, or a hydrate thereof, is isolated, e.g., by filtration and/or centrifugation and vacuum drying (e.g., at about 50° C.). It will be appreciated that solid and/or crystalline forms can form spontaneously, and/or can be induced to form, such as by crystal seeding or altering the conditions to which a mixture is exposed (as when a mixture is cooled, for example). “Precipitating,” as used herein, is intended to include both the situation when formation of a solid and/or crystalline form is induced and the situation when formation of a solid and/or crystalline form occurs spontaneously.

As used herein, “solvent” refers to a liquid that serves as a medium for a chemical reaction or other procedure in which compounds are being manipulated (e.g., crystallization). Typically, the solvent in the methods disclosed herein is an organic solvent or water, or a combination thereof. Examples of organic solvents include polar, protic solvents (e.g., an alcohol such as methanol, ethanol, butanol, such as tert-butanol), polar aprotic solvents (e.g., acetonitrile, dimethylformamide, tetrahydrofuran, ethyl acetate, acetone, methyl ethyl ketone) or nonpolar solvents (e.g., diethyl ether). In some embodiments, the solvent is tetrahydrofuran, a mixture (e.g., about 19:1 mixture) of acetonitrile and water, acetone or ethyl acetate.

### Pharmaceutical Compositions and Combinations

Compounds of the present disclosure are typically used in a pharmaceutical composition (e.g., a pharmaceutical composition comprising a compound of the present disclosure, or a pharmaceutically acceptable salt thereof, or a hydrate of the foregoing, and one or more pharmaceutically acceptable carriers). A “pharmaceutically acceptable carrier” refers to media generally accepted in the art for the delivery of biologically active agents to animals, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like, and combinations thereof, as would be known to those skilled in the art (see, for example, Allen, L. V., Jr. et al., Remington: The Science and Practice of Pharmacy (2 Volumes), 22nd Edition, Pharmaceutical Press (2012).

In one aspect, provided herein is a pharmaceutical composition comprising a compound of the present disclosure (e.g., a compound of structural formula I, or a pharmaceutically acceptable salt thereof, or a hydrate of the foregoing) (e.g., a therapeutically effective amount of a compound of the present disclosure), and a pharmaceutically acceptable carrier. In a further embodiment, the composition comprises at least two pharmaceutically acceptable carriers, such as those described herein. For purposes of the present disclosure, unless designated otherwise, solvates are generally considered compositions. Preferably, pharmaceutically acceptable carriers are sterile.

A pharmaceutical composition can be formulated for particular routes of administration such as oral administration, parenteral administration (e.g., intravenous administration) and rectal administration, etc. In addition, the pharmaceutical compositions of the present disclosure can be made up in a solid form (including, without limitation, capsules, tablets, pills, granules, powders or suppositories), or in a liquid form (including, without limitation, solutions, suspensions or emulsions). The pharmaceutical compositions can be subjected to conventional pharmaceutical operations, such as sterilization, and/or can contain conventional inert diluents, lubricating agents, or buffering agents, as well as adjuvants, such as preservatives, stabilizers, wetting agents, emulsifiers and buffers, etc. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol;
c) binders, e.g., magnesium aluminum silicate, starch paste, gelatin, tragacanth, methylcellulose, sodium carboxymethylcellulose and/or polyvinylpyrrolidone;
d) disintegrants, e.g., starches, agar, alginic acid or its sodium salt, or effervescent mixtures; and
e) absorbents, colorants, flavors and sweeteners.
   Tablets may be either film-coated or enteric-coated according to methods known in the art.

In some aspects, the composition or unit dosage form is formulated for oral administration, e.g., in the form of a capsule.

Suitable compositions for oral administration include a compound of the present disclosure (e.g., a compound of structural formula I, or a pharmaceutically acceptable salt thereof, or a hydrate of the foregoing) in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions comprise a compound of the present disclosure (e.g., a compound of structural formula I, or a pharmaceutically acceptable salt thereof, or a hydrate of the foregoing) in the form of an aqueous isotonic solution or suspension, and certain suppositories comprising a compound of the present disclosure (e.g., a compound of structural formula I, or a pharmaceutically acceptable salt thereof, or a hydrate of the foregoing) are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include a compound of the present disclosure (e.g., a compound of structural formula I, or a pharmaceutically acceptable salt thereof, or a hydrate of the foregoing) with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Suitable compositions comprising a compound of the present disclosure (e.g., a compound of structural formula I, or a pharmaceutically acceptable salt thereof, or a hydrate of the foregoing) for topical application, e.g., to the skin and eyes, include aqueous solutions, suspensions, ointments, creams, gels or sprayable formulations, e.g., for delivery by aerosol or the like. Such topical delivery systems will, in particular, be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein, a topical application may also pertain to an inhalation or to an intranasal application. A composition suitable for inhalation or intranasal administration may be conveniently delivered in the form of a dry powder (either alone, as a mixture, for example a dry blend with lactose, or a mixed component particle, for example, with phospholipids) from a dry powder inhaler, or an aerosol spray presentation from a pressurised container, pump, spray, atomizer or nebuliser, with or without the use of a suitable propellant.

In some particular aspects, provided is a pharmaceutical composition comprising a compound of the present disclosure; and silicified microcrystalline cellulose; or croscarmellose sodium; or sodium stearyl fumarate. For example, in some aspects, the pharmaceutical composition comprises a compound of the present disclosure; silicified microcrystalline cellulose; croscarmellose sodium; and sodium stearyl fumarate. In some aspects, a pharmaceutical composition comprises from about 5% to about 50% (e.g., about 35%) by weight of a compound of the present disclosure, based on the molecular weight of the compound of structural formula I as a free base.

When a weight (or percent by weight) is “based on the molecular weight of the compound of structural formula I as a free base,” the indicated weight (or percent by weight) refers to the corresponding weight (or percent by weight) of the compound of structural formula I, calculated as a free base, regardless of whether the compound present is in its free base form or other form, e.g., as a pharmaceutically acceptable salt, such as a mesylate salt. It will be appreciated that if the compound of structural formula I is present in its free base form, the indicated weight of the compound of structural formula I will correspond to the actual weight of the compound of structural formula present. If, however, the compound of structural formula I is present as a mesylate salt, the indicated weight of the compound of structural formula I is the weight calculated from the actual weight of the compound of structural formula I present by dividing the actual weight of the compound of structural formula I present by its molecular weight and multiplying the resulting quotient by the molecular weight of the compound of structural form as a free base, taking into account the molar ratio of the compound of structural formula I present to the compound of structural formula I as a free base. The molecular weight of the compound of structural formula I as a free base is 543.4 g/mol.

In some aspects of a pharmaceutical composition comprising silicified microcrystalline cellulose, the pharmaceutical composition comprises from about 25% to about 50% (e.g., about 30%) by weight silicified microcrystalline cellulose. In aspects of any of the foregoing aspects of a pharmaceutical composition comprising silicified microcrystalline cellulose, the silicified microcrystalline cellulose has an average particle size by laser diffraction of about 125 μm. In aspects of any of the foregoing aspects of a pharmaceutical composition comprising silicified microcrystalline cellulose, the silicified microcrystalline cellulose has a bulk density of from about 0.25 to about 0.37 g/mL.

In some aspects of a pharmaceutical composition comprising croscarmellose sodium, the pharmaceutical composition comprises from about 1% to about 10% (e.g., about 5%) by weight croscarmellose sodium.

In some aspects of a pharmaceutical composition comprising sodium stearyl fumarate, the pharmaceutical composition comprises from about 0.1% to about 5% (e.g., about 0.5%) by weight sodium stearyl fumarate.

In some aspects, a pharmaceutical composition comprises or further comprises mannitol (e.g., D-mannitol), or a pharmaceutically acceptable salt thereof, e.g., from about 25% to about 50% (e.g., about 30%) by weight mannitol (e.g., D-mannitol), or a pharmaceutically acceptable salt thereof. In aspects of the foregoing aspects of a pharmaceutical composition comprising mannitol, or a pharmaceutically acceptable salt thereof, the mannitol, or a pharmaceutically acceptable salt thereof, has a d₁₀ of about 40 μm, a d₅₀ of about 130 μm and a d₉₀ of about 200 μm.

A compound of the present disclosure (e.g., a compound of Formula I, or a subformula thereof, or a pharmaceutically acceptable salt of the foregoing) is typically formulated into pharmaceutical dosage forms to provide an easily controllable dosage of the drug and to give the patient an elegant and easily handleable product. Accordingly, some aspects provide a unit dosage form comprising a pharmaceutical composition or combination described herein. In some aspects, the unit dosage form comprises from about 5 mg to about 250 mg, e.g., about 7 mg, about 28 mg, about 50 mg, about 100 mg, about 200 mg, of the compound of structural formula I, or a pharmaceutically acceptable salt thereof, or hydrate of the foregoing, based on the molecular weight of the compound of structural formula I as a free base. In a particular aspect, the unit dosage form comprises about 50 mg of the compound of structural formula I, or a pharmaceutically acceptable salt thereof, or hydrate of the foregoing, based on the molecular weight of the compound of structural formula I as a free base. In another particular aspect, the unit dosage form comprises about 100 mg of the compound of structural formula I, or a pharmaceutically acceptable salt thereof, or hydrate of the foregoing, based on the molecular weight of the compound of structural formula I as a free base. In yet another particular aspect, the unit dosage form comprises about 200 mg of the compound of structural formula I, or a pharmaceutically acceptable salt thereof, or hydrate of the foregoing, based on the molecular weight of the compound of structural formula I as a free base.

The dosage regimen for the compounds of the present disclosure will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration; the renal and hepatic function of the patient; and the effect desired. Compounds of the present disclosure (e.g., a compound of structural formula I, or a pharmaceutically acceptable salt thereof, or a hydrate of the foregoing) may be administered in a single daily dose, or the total daily dosage may be administered in divided doses, e.g., two, three, or four times daily.

Also provided herein are pharmaceutical compositions and dosage forms having certain stability characteristics. Thus, in some aspects, a pharmaceutical composition or dosage form described herein contains less than 3% by weight water, as measured by Karl Fischer titration after four weeks at about 40° C. and about 75% relative humidity in a closed container. In addition, or alternatively, in some aspects, a pharmaceutical composition described herein has a purity of at least 95%, as measured by high-performance liquid chromatography (HPLC) after four weeks at about 40° C. and about 75% relative humidity in a closed container. In addition, or alternatively, in some aspects, a pharmaceutical composition described herein contains total impurities of less than 1% (e.g., less than 0.75%), as measured by HPLC after four weeks at about 40° C. and about 75% relative humidity in a closed container.

The present disclosure further provides anhydrous pharmaceutical compositions and dosage forms comprising a compound of the present disclosure (e.g., a compound of structural formula I, or a pharmaceutically acceptable salt thereof, or a hydrate of the foregoing), since water may facilitate the degradation of certain compounds. Anhydrous pharmaceutical compositions and dosage forms of the disclosure can be prepared using anhydrous or low moisture-containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The present disclosure further provides pharmaceutical compositions and dosage forms that comprise one or more agents that reduce the rate by which a compound of the present disclosure (e.g., a compound of structural formula I, or a pharmaceutically acceptable salt thereof, or a hydrate of the foregoing) as an active ingredient will decompose. Such agents, which are referred to herein as “stabilizers,” include, but are not limited to, antioxidants such as ascorbic acid, pH buffers, or salt buffers, etc.

In certain instances, it may be advantageous to administer a compound of the present disclosure (e.g., a compound of structural formula I, or a pharmaceutically acceptable salt thereof, or a hydrate of the foregoing) in combination with one or more additional therapeutically active agents. For example, it may be advantageous to administer a compound of the present disclosure (e.g., a compound of structural formula I, or a pharmaceutically acceptable salt thereof, or a hydrate of the foregoing) in combination with one or more additional therapeutically active agents independently selected from an anti-cancer agent (e.g., chemotherapeutic agent), anti-allergic agent, anti-emetic, pain reliever, immunomodulator or cytoprotective agent to treat cancer. In some embodiments, a compound of the present disclosure is administered in combination with one or more therapeutically active agents to treat a TNK1-mediated disease, disorder or condition, e.g., a gastrointestinal disorder, an inflammatory disorder, tissue injury, multi-organ dysfunction syndrome (MODS), sepsis, an autoimmune disorder, a disease, disorder or condition of the microbiome or a disease, disorder or condition resulting from a trauma and/or intestinal injury.

The term “combination therapy” refers to the administration of two or more therapeutically active agents to treat a disease, disorder or condition described herein. Such administration encompasses co-administration of the therapeutically active agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Such administration also encompasses use of each type of therapeutically active agent in a sequential manner, either at approximately the same time or at different times. A compound of the present disclosure (e.g., a compound of structural formula I, or a pharmaceutically acceptable salt thereof, or a hydrate of the foregoing) and an additional therapeutically active agent(s) can be administered via the same administration route or via different administration routes. Powders and/or liquids may be reconstituted or diluted to a desired dose prior to administration. Typically, the treatment regimen will provide beneficial effects of the drug combination in treating the diseases, conditions or disorders described herein.

In some embodiments, the methods for combination therapies described herein provide a therapeutically active agent known to modulate other pathways, or other components of the same pathway, or even overlapping sets of target enzymes than the pathways, components or sets modulated by a compound of the present disclosure. In one aspect, such therapy includes, but is not limited to, combinations of a compound of the present disclosure and a chemotherapeutic agent(s), therapeutic antibody(ies), and/or radiation treatment that provide a synergistic or additive therapeutic effect.

Compositions for use in combination therapies will either be formulated together as a pharmaceutical combination, or provided for separate administration (e.g., associated in a kit). Accordingly, a further embodiment is a pharmaceutical combination comprising a compound of the present disclosure (e.g., a compound of structural formula I, or a pharmaceutically acceptable salt thereof, or a hydrate of the foregoing) (e.g., a therapeutically effective amount of a compound of the present disclosure), and one or more additional therapeutically active agents (e.g., a therapeutically effective amount of one or more additional therapeutically active agents). A pharmaceutical combination can further comprise one or more pharmaceutically acceptable carriers, such as one or more of the pharmaceutically acceptable carriers described herein.

Examples of therapies for use in combination with a compound of the present disclosure (e.g., in combination therapy, in a pharmaceutical combination) include standard of care therapies (e.g., standard of care agents). Standard of care therapies are therapies that a clinician should use for a certain type of patient, illness and/or clinical circumstance. For example, a non-limiting example of a standard of care agent for pancreatic cancer is gemcitabine. Non-limiting examples of standard of care agents for colorectal cancer are FOLFIRINOX (a chemotherapy regimen made up of folinic acid, fluorouracil, irinotecan and oxaliplatin). Often, organizations such as National Comprehensive Cancer Network (NCCN) publish guidelines and/or treatment algorithms setting forth best practices for treatment of certain patients, illnesses and/or clinical circumstances. See nccn.org. These guidelines often establish, set forth and/or summarize standard of care therapies.

Radiation therapy can be administered in combination with a compound of the present disclosure in some embodiments. Exemplary radiation therapies include external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At211, I131, I125, Y90, Re186, Re188, Sm153, Bi212, P32, and radioactive isotopes of Lu). Suitable radiation sources for use as a cell conditioner of the present invention include both solids and liquids. By way of non-limiting example, the radiation source can be a radionuclide, such as I125, I131, Yb169, Ir192 as a solid source, I125 as a solid source, or other radionuclides that emit photons, beta particles, gamma radiation, or other therapeutic rays. The radioactive material can also be a fluid made from any solution of radionuclide(s), e.g., a solution of I125 or I131, or a radioactive fluid can be produced using a slurry of a suitable fluid containing small particles of solid radionuclides, such as Au198, Y90. Moreover, the radionuclide(s) can be embodied in a gel or radioactive micro spheres.

Without being limited by any theory, a compound of the present disclosure can render abnormal cells more sensitive to treatment with radiation for purposes of killing and/or inhibiting the growth of such cells. Accordingly, some embodiments include a method for sensitizing abnormal cells in a mammal to treatment with radiation which comprises administering to the mammal an amount of a compound of the present disclosure, which amount is effective at sensitizing abnormal cells to treatment with radiation. The amount of a compound of the present disclosure in this method can be determined according to means for ascertaining effective amounts of such compounds. In some embodiments, standard of care therapy includes radiation therapy.

Non-limiting examples of chemotherapeutic agents for use in combination with a compound of the present disclosure (e.g., in combination therapy, in a pharmaceutical combination) include capecitabine (Xeloda®), N4-pentoxycarbonyl-5-deoxy-5-fluorocytidine, carboplatin (Paraplatin®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), doxorubicin hydrochloride (Adriamycin®, Rubex®), fludarabine phosphate (Fludara®), 5-fluorouracil (Adrucil®, Efudex®), gemcitabine (difluorodeoxycitidine), irinotecan (Camptosar®), L-asparaginase (ELSPAR®), 6-mercaptopurine (Purinethol®), methotrexate (Folex®), pentostatin, 6-thioguanine, thiotepa, and topotecan hydrochloride for injection (Hycamptin®). A further example is bortezomib. Yet further examples include gemcitabine, nabpaclitaxel, erlotinib, fluorouracil and FOLFIRINOX (a chemotherapy regimen made up of folinic acid, fluorouracil, irinotecan and oxaliplatin), or any combination of two or more of the foregoing, e.g., to treat pancreatic cancer (e.g., advanced pancreatic cancer, pancreatic ductal adenocarcinoma).

Anti-cancer agents of particular interest for use in combination with the compounds of the present disclosure include:

Purine antimetabolites and/or inhibitors of de novo purine synthesis: pemetrexed (Alimta®), gemcitabine (Gemzar®), 5-fluorouracil (Adrucil®, Carac® and Efudex®), methotrexate (Trexall®), capecitabine (Xeloda®), floxuridine (FUDR®), decitabine (Dacogen®), azacitidine (Vidaza® and Azadine®), 6-mercaptopurine (Purinethol®), cladribine (Leustatin®, Litak® and Movectro®), fludarabine (Fludara®), pentostatin (Nipent®), nelarabine (Arranon®), clofarabine (Clolar® and Evoltra®), and cytarabine (Cytosar®). Anti-angiogenesis agents include, for example, MMP-2 (matrix-metalloproteinase 2) inhibitors, rapamycin, temsirolimus (CCI-779), everolimus (RAD001), sorafenib, sunitinib, and bevacizumab. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Embodiments of MMP-2 and MMP-9 inhibitors include those that have little or no activity inhibiting MMP-1. Other embodiments include those that selectively inhibit MMP-2 and/or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors useful in some embodiments are AG-3340, RO 323555, and RS 13-0830.

Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used.

In other embodiments, agents useful in methods for combination therapy with a compound of the present disclosure include, but are not limited to: Erlotinib, Afatinib, Iressa, GDC0941, MLN1117, BYL719 (Alpelisib), BKM120 (Buparlisib), CYT387, GLPG0634, Baricitinib, Lestaurtinib, momelotinib, Pacritinib, Ruxolitinib, TG101348, Crizotinib, tivantinib, AMG337, cabozantinib, foretinib, onartuzumab, NVP-AEW541, Dasatinib, Ponatinib, saracatinib, bosutinib, trametinib, selumetinib, cobimetinib, PD0325901, RO5126766, Axitinib, Bevacizumab, Bostutinib, Cetuximab, Crizotinib, Fostamatinib, Gefitinib, Imatinib, Lapatinib, Lenvatinib, Ibrutinib, Nilotinib, Panitumumab, Pazopanib, Pegaptanib, Ranibizumab, Ruxolitinib, Sorafenib, Sunitinib, SU6656, Trastuzumab, Tofacitinib, Vandetanib, Vemurafenib, Irinotecan, Taxol, Docetaxel, Rapamycin or MLN0128.

B-cell receptor signaling antagonists (e.g., Bruton's tyrosine kinase (BTK) inhibitors): Ibrutinib and venetoclax.

Bromodomain inhibitors. A bromodomain inhibitor inhibits at least one bromodomain protein, such as Brd2, Brd3, Brd4 and/or BrdT, for example Brd4. In some of these embodiments, the bromodomain inhibitor is JQ-1 (Nature 2010 Dec. 23; 468(7327):1067-73), BI2536 (ACS Chem. Biol. 2014 May 16; 9(5):1160-71; Boehringer Ingelheim), TG101209 (ACS Chem. Biol. 2014 May 16; 9(5):1160-71), OTX015 (Mol. Cancer Ther. November 201312; C244; Oncoethix), IBET762 (J Med Chem. 2013 Oct. 10; 56(19):7498-500; GlaxoSmithKline), IBET151 (Bioorg. Med. Chem. Lett. 2012 Apr. 15; 22(8):2968-72; GlaxoSmithKline), PFI-1 (J. Med. Chem. 2012 Nov. 26; 55(22):9831-7; Cancer Res. 2013 Jun. 1; 73(11):3336-46; Structural Genomics Consortium) of CPI-0610 (Constellation Pharmaceuticals). In some embodiments, the bromodomain inhibitor is TG101209, BI2536, OTX015, C244, IBET762, IBET151, or PFI-1.

Histone deacetylase (HDAC) inhibitors. A HDAC inhibitor inhibits at least one HDAC protein. HDAC proteins may be grouped into classes based on homology to yeast HDAC proteins with Class I made up of HDAC1, HDAC2, HDAC3 and HDAC 8; Class IIa made up of HDAC4, HDAC5, HDAC7 and HDAC 9; Class IIb made up of HDAC6 and HDAC10; and Class IV made up of HDAC11. In some of these embodiments, the HDAC inhibitor is trichostatin A, vorinostat (Proc. Natl. Acad. Sci. U.S.A. 1998 Mar. 17; 95(6):3003-7), givinostat, abexinostat (Mol. Cancer Ther. 2006 May; 5(5):1309-17), belinostat (Mol. Cancer Ther. 2003 August; 2(8):721-8), panobinostat (Clin. Cancer Res. 2006 Aug. 1; 12(15):4628-35), resminostat (Clin. Cancer Res. 2013 Oct. 1; 19(19):5494-504), quisinostat (Clin. Cancer Res. 2013 Aug. 1; 19(15):4262-72), depsipeptide (Blood. 2001 Nov. 1; 98(9):2865-8), entinostat (Proc. Natl. Acad. Sci. U.S.A. 1999 Apr. 13; 96(8):4592-7), mocetinostat (Bioorg. Med. Chem. Lett. 2008 Feb. 1; 18(3):106771) or valproic acid (EMBO J. 2001 Dec. 17; 20(24):6969-78). For example, in some embodiments the HDAC inhibitor is panobinostat, vorinostat, MS275, belinostat, or LBH589. In some embodiments, the HDAC inhibitor is panobinostat or SAHA.

In embodiments, a compound of the present disclosure is administered in combination with an epidermal growth factor receptor tyrosine kinase (EGFR) inhibitor. Examples of EGFR inhibitors include erlotinib, osimertinib, cetuximab, gefitinib, necitumumab, lapatinib, neratinib, panitumumab, vandetanib, and necitumumab. A combination of a compound as described herein and an EGFR inhibitor may be useful, for example, in the treatment of cancers that are related to EGFR dysregulation, such as non-small-cell lung cancer (NSCLC), pancreatic cancer, breast cancer, and colon cancer. EGFR may be dysregulated, for example, due to activating mutations in exons 18, 19, 20, or 21. In particular embodiments, the EGFR inhibitor is erlotinib or osimertinib. In particular embodiments, the combination of a compound as described herein and an EGFR inhibitor is used to treat EGFR-mutated NSCLC. In particular embodiments, the combination of a compound as described herein and an EGFR inhibitor is used to treat an EGFR inhibitor-resistant cancer, and the compound as described herein sensitized the cancer to the EGFR inhibitor.

EGFR antibodies: cetuximab (Erbitux®).

MTAP inhibitors: (3R,4S)-1-((4-amino-5H-pyrrolo[3,2-d]pyrimidin-7-yl)methyl)-4-((methylthio)methyl)pyrrolidin-3-ol (MT-DADMe-Immucillin-A, CAS 653592-04-2).

Methylthioadenosine: ((2R,3R,4S,5S)-2-(6-amino-9H-purin-9-yl)-5-((methylthio)methyl)tetrahydrofuran-3,4-diol, CAS 2457-80-9).

Epidermal growth factor receptor (EGFR) inhibitors: erlotinib hydrochloride (Tarceva®) and gefitnib (Iressa®).

EGFR antibodies: cetuximab (Erbitux®).

MET inhibitors: capmatinib (INC280, CAS 1029712-80-8).

Platelet-derived growth factor (PDGF) receptor inhibitors: imatinib (Gleevec®); linifanib (N-[4-(3-amino-1H-indazol-4-yl)phenyl]-N′-(2-fluoro-5-methylphenyl)urea, also known as ABT 869, available from Genentech); sunitinib malate (Sutent®); quizartinib (AC220, CAS 950769-58-1); pazopanib (Votrient®); axitinib (Inlyta®); sorafenib (Nexavar®); vargatef (BIBF1120, CAS 928326-83-4); telatinib (BAY57-9352, CAS 332012-40-5); vatalanib dihydrochloride (PTK787, CAS 212141-51-0); and motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470).

Phosphoinositide 3-kinase (PI3K) inhibitors: 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 4-(trifluoromethyl)-5-(2,6-dimorpholinopyrimidin-4-yl)pyridin-2-amine (also known as BKM120 or NVP-BKM120, and described in PCT Publication No. WO 2007/084786); alpelisib (BYL719): (5Z)-5-[[4-(4-Pyridinyl)-6-quinolinyl]methylene]-2,4-thiazolidinedione (GSK1059615, CAS 958852-01-2); 5-[8-methyl-9-(1-methylethyl)-2-(4-morpholinyl)-9H-purin-6-yl]-2-pyrimidinamine (VS-5584, CAS 1246560-33-7) and everolimus (AFINITOR®).

Cyclin-dependent kinase (CDK) inhibitors: ribociclib (LEE011, CAS 1211441-98-3); aloisine A; alvocidib (also known as flavopiridol or HMR-1275, 2-(2-chlorophenyl)-5,7-dihydroxy-8-[(3S,4R)-3-hydroxy-1-methyl-4-piperidinyl]-4-chromenone, and described in U.S. Pat. No. 5,621,002); crizotinib (PF-02341066, CAS 877399-52-5); 2-(2-chlorophenyl)-5,7-dihydroxy-8-[(2R,3S)-2-(hydroxymethyl)-1-methyl-3-pyrrolidinyl]-4H-1-benzopyran-4-one, hydrochloride (P276-00, CAS 920113-03-7); 1-methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265, CAS 927880-90-8); indisulam (E7070); roscovitine (CYC202); 6-acetyl-8-cyclopentyl-5-methyl-2-(5-piperazin-1-yl-pyridin-2-ylamino)-8H-pyrido[2,3-d]pyrimidin-7-one, hydrochloride (PD0332991); dinaciclib (SCH727965); N-[5-[[(5-tert-butyloxazol-2-yl)methyl]thio]thiazol-2-yl]piperidine-4-carboxamide (BMS 387032, CAS 345627-80-7); 4-[[9-chloro-7-(2,6-difluorophenyl)-5H-pyrimido[5,4-d][2]benzazepin-2-yl]amino]-benzoic acid (MLN8054, CAS 869363-13-3); 5-[3-(4,6-difluoro-1H-benzimidazol-2-yl)-1H-indazol-5-yl]-N-ethyl-4-methyl-3-pyridinemethanamine (AG-024322, CAS 837364-57-5); 4-(2,6-dichlorobenzoylamino)-1H-pyrazole-3-carboxylic acid N-(piperidin-4-yl)amide (AT7519, CAS 844442-38-2); 4-[2-methyl-1-(1-methylethyl)-1H-imidazol-5-yl]-N-[4-(methylsulfonyl)phenyl]-2-pyrimidinamine (AZD5438, CAS 602306-29-6); palbociclib (PD-0332991); and (2R,3R)-3-[[2-[[3-[[S(R)]-S-cyclopropylsulfonimidoyl]-phenyl]amino]-5-(trifluoromethyl)-4-pyrimidinyl]oxy]-2-butanol (BAY 10000394).

p53-MDM2 inhibitors: (S)-1-(4-chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, (S)-5-(5-chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, [(4S,5R)-2-(4-tert-butyl-2-ethoxyphenyl)-4,5-bis(4-chlorophenyl)-4,5-dimethylimidazol-1-yl]-[4-(3-methylsulfonylpropyl)piperazin-1-yl]methanone (RG7112), 4-[[(2R,3S,4R,5S)-3-(3-chloro-2-fluorophenyl)-4-(4-chloro-2-fluorophenyl)-4-cyano-5-(2,2-dimethylpropyl)pyrrolidine-2-carbonyl]amino]-3-methoxybenzoic acid (RG7388), SAR299155, 2-((3R,5R,6S)-5-(3-chlorophenyl)-6-(4-chlorophenyl)-1-((S)-1-(isopropylsulfonyl)-3-methylbutan-2-yl)-3-methyl-2-oxopiperidin-3-yl)acetic acid (AMG232), {(3R,5R,6S)-5-(3-chlorophenyl)-6-(4-chlorophenyl)-1-[(2S,3S)-2-hydroxy-3-pentanyl]-3-methyl-2-oxo-3-piperidinyl}acetic acid (AM-8553), (+)-4-[4,5-bis(4-chlorophenyl)-2-(2-isopropoxy-4-methoxy-phenyl)-4,5-dihydro-imidazole-1-carbonyl]-piperazin-2-one (Nutlin-3), 2-methyl-7-[phenyl(phenylamino)methyl]-8-quinolinol (NSC 66811), 1-N-[2-(1H-indol-3-yl)ethyl]-4-N-pyridin-4-ylbenzene-1,4-diamine (JNJ-26854165), 4-[4,5-bis(3,4-chlorophenyl)-2-(2-isopropoxy-4-methoxy-phenyl)-4,5-dihydro-imidazole-1-carboxyl]-piperazin-2-one (Caylin-1), 4-[4,5-bis(4-trifluoromethyl-phenyl)-2-(2-isopropoxy-4-methoxy-phenyl)-4,5-dihydro-imidazole-1-carboxyl]-piperazin-2-one (Caylin-2), 5-[[3-dimethylamino)propyl]amino]-3,10-dimethylpyrimido[4,5-b]quinoline-2,4(3H,10H)-dione dihydrochloride (HLI373) and trans-4-iodo-4′-boranyl-chalcone (SC204072).

Mitogen-activated protein kinase (MEK) inhibitors: XL-518 (also known as GDC-0973, CAS No. 1029872-29-4, available from ACC Corp.); selumetinib (5-[(4-bromo-2-chlorophenyl)amino]-4-fluoro-N-(2-hydroxyethoxy)-1-methyl-1H-benzimidazole-6-carboxamide, also known as AZD6244 or ARRY 142886, described in PCT Publication No. WO 2003/077914); 2-[(2-chloro-4-iodophenyl)amino]-N-(cyclopropylmethoxy)-3,4-difluoro-benzamide (also known as CI-1040 or PD184352 and described in PCT Publication No. WO 2000/035436); N-[(2R)-2,3-dihydroxypropoxy]-3,4-difluoro-2-[(2-fluoro-4-iodophenyl)amino]-benzamide (also known as PD0325901 and described in PCT Publication No. WO 2002/006213); 2,3-bis[amino[(2-aminophenyl)thio]methylene]-butanedinitrile (also known as U0126 and described in U.S. Pat. No. 2,779,780); N-[3,4-difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO 2007/014011); (3S,4R,5Z,8S,9S,11E)-14-(ethylamino)-8,9,16-trihydroxy-3,4-dimethyl-3,4,9;19-tetrahydro-1H-2-benzoxacyclotetradecine-1,7(8H)-dione](also known as E6201 and described in PCT Publication No. WO 2003/076424); 2′-amino-3′-methoxyflavone (also known as PD98059 available from Biaffin GmbH & Co., KG, Germany); (R)-3-(2,3-dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); trametinib dimethyl sulfoxide (GSK-1120212, CAS 1204531-25-80); 2-(2-fluoro-4-iodophenylamino)-N-(2-hydroxyethoxy)-1,5-dimethyl-6-oxo-1,6-dihydropyridine-3-carboxamide (AZD 8330); 3,4-difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655); and 5-[(4-bromo-2-fluorophenyl)amino]-4-fluoro-N-(2-hydroxyethoxy)-1-methyl-1H-benzimidazole-6-carboxamide (MEK162).

B-RAF inhibitors: regorafenib (BAY73-4506, CAS 755037-03-7); tuvizanib (AV951, CAS 475108-18-0); vemurafenib (ZELBORAF®, PLX-4032, CAS 918504-65-1); encorafenib (also known as LGX818); 1-methyl-5-[[2-[5-(trifluoromethyl)-1H-imidazol-2-yl]-4-pyridinyl]oxy]-N-[4-(trifluoromethyl)phenyl-1H-benzimidazol-2-amine (RAF265, CAS 927880-90-8); 5-[1-(2-hydroxyethyl)-3-(pyridin-4-yl)-1H-pyrazol-4-yl]-2,3-dihydroinden-1-one oxime (GDC-0879, CAS 905281-76-7); 5-[2-[4-[2-(dimethylamino)ethoxy]phenyl]-5-(4-pyridinyl)-1H-imidazol-4-yl]-2,3-dihydro-1H-inden-1-one oxime (GSK2118436 or SB590885); (+/−)-methyl (5-(2-(5-chloro-2-methylphenyl)-1-hydroxy-3-oxo-2,3-dihydro-1H-isoindol-1-yl)-1H-benzimidazol-2-yl)carbamate (also known as XL-281 and BMS908662), dabrafenib (TAFINLAR®), and N-(3-(5-chloro-1H-pyrrolo[2,3-b]pyridine-3-carbonyl)-2,4-difluorophenyl)propane-1-sulfonamide (also known as PLX4720).

ALK inhibitors: crizotinib (XALKORI®).

PIM kinase inhibitors:

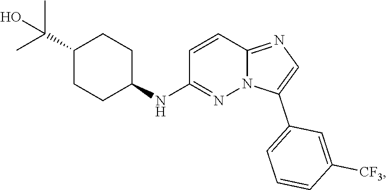 [SMILES: CC(C)(O)[C@H]1CC[C@H](Nc2ccc3ncc(-c4cccc(C(F)(F)F)c4)n3n2)CC1]

or a pharmaceutically acceptable salt thereof.

Proteasome inhibitors: bortezomib (VELCADE®), N-5-benzyloxycarbonyl-Ile-Glu(O-tert-butyl)-Ala-leucinal (PSI), carfilzomib and ixazomib, marizomib (NPI-0052), delanzomib (CEP-18770), and O-methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (oprozomib, ONX-0912, PR-047) (e.g., bortezomib). An RNAi screen identified TNK1 as a potential modulator of proteasome inhibitor sensitivity in myeloma. Zhu et al., Blood (2011) 117 (14): 3847-3857. In some embodiments, a compound of the present disclosure (e.g., a compound of Formula I, or a subformula thereof, or a pharmaceutically acceptable salt of the foregoing) is administered in combination with a proteasome inhibitor described herein, such as bortezomib, e.g., for the treatment of multiple myeloma.

Further non-limiting examples of therapeutically active agents that can be used in combinations with a compound of the present disclosure include chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin as well as a host of chemotherapeutic agents. Non-limiting examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex®, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above.

In some embodiments, the chemotherapeutic agent is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

More non-limiting examples of chemotherapeutic agents for use in combination with a compound of the present disclosure (e.g., in combination therapy, in a pharmaceutical combination) include capecitabine (Xeloda®), N4-pentoxycarbonyl-5-deoxy-5-fluorocytidine, carboplatin (Paraplatin®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), doxorubicin hydrochloride (Adriamycin®, Rubex®), fludarabine phosphate (Fludara®), 5-fluorouracil (Adrucil®, Efudex®), gemcitabine (difluorodeoxycitidine), irinotecan (Camptosar®), L-asparaginase (ELSPAR®), 6-mercaptopurine (Purinethol®), methotrexate (Folex®), pentostatin, 6-thioguanine, thiotepa, and topotecan hydrochloride for injection (Hycamptin®).

Further non-limiting examples of commonly prescribed anti-cancer drugs include Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomeRASe inhibitor RFS 2000; difluoromethylornithine (DMFO).

Non-limiting examples of therapeutically active agents that can be used in combinations with a compound of the present disclosure include mTOR inhibitors. Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-inner salt (SEQ ID NO: 1482) (SF1126, CAS 936487-67-1), and XL765.

In certain other embodiments, a method for treating cancer is provided, the method comprising administering an effective amount of a compound of the present disclosure and a CDK inhibitor to a subject in need thereof.

In embodiments, the CDK inhibitor is a CDK2, CDK4, CDK6, CDK7, CDK8, CDK9, CDK10, and/or CDK11 inhibitor. In some embodiments, the CDK inhibitor is a CDK7, CDK9 inhibitor, or both. In some embodiments, the CDK inhibitor is dinaciclib (ACS Med. Chem. Lett. 2010 May 17; 1(5):204-8; Mol. Cancer Ther. 2010 August; 9(8):2344-53; Merck, Sharp and Dohme), AT7519 (J. Med. Chem. 2008 Aug. 28; 51(16):4986-99; Astex Pharmaceutical) or palbociclib (J. Med. Chem. 2005 Apr. 7; 48(7):2388-406; Pfizer). In certain embodiments, the CDK inhibitor is a CDK9 inhibitor, such as alvocidib. The alvocidib may be administered as the free bases, as a pharmaceutically acceptable salt or as a prodrug. In certain embodiments, the CDK9 inhibitor is alvocidib. In other embodiments, the CDK9 inhibitor is a pharmaceutically acceptable salt of alvocidib. In other embodiments, the CDK9 inhibitor is a prodrug of alvocidib. Prodrugs of alvocidib include those disclosed in WO 2016/187316, the full disclosure of which is hereby incorporated by reference in its entirety.

In one embodiment, a compound of the present disclosure is administered to a subject in need thereof in combination with an ATR inhibitor, such as AZD6738 or VX-970. The administration may be before, concurrently or after administration of the ATR inhibitor. In one specific embodiment, a compound of the present disclosure is administered to a subject in need thereof in combination with an ATR inhibitor, such as AZD6738 or VX-970 for treatment of non-small cell lung cancer. In some of the foregoing embodiments, the ATR inhibitor is AZD6738. In some of the foregoing embodiments, the ATR inhibitor is VX-970. In some of the foregoing embodiments, the ATR inhibitor is a combination of AZD6738 and VX-970.

Some patients may experience allergic reactions to compounds of the present disclosure and/or other therapeutically active agent(s) (e.g., anti-cancer agent(s)) during or after administration. Therefore, anti-allergic agents can be administered in combination with compounds of the present disclosure and/or other therapeutically active agent(s) (e.g., anti-cancer agent(s)) to minimize the risk of an allergic reaction. Suitable anti-allergic agents include corticosteroids (Knutson, S., et al., PLoS One, DOI:10.1371/journal.pone.0111840 (2014)), such as dexamethasone (e.g., DECADRON®), beclomethasone (e.g., BECLOVENT®), hydrocortisone (also known as cortisone, hydrocortisone sodium succinate, hydrocortisone sodium phosphate, sold under the tradenames ALA-CORT®, hydrocortisone phosphate, SOLU-CORTEF®, HYDROCORT ACETATE® and LANACORT®), prednisolone (sold under the tradenames DELTA-CORTEL®, ORAPRED®, PEDIAPRED® and PRELONE®), prednisone (sold under the tradenames DELTASONE®, LIQUID RED®, METICORTEN® and ORASONE®), methylprednisolone (also known as 6-methylprednisolone, methylprednisolone acetate, methylprednisolone sodium succinate, sold under the tradenames DURALONE®, MEDRALONE®, MEDROL®, M-PREDNISOL® and SOLU-MEDROL®); antihistamines, such as diphenhydramine (e.g., BENADRYL®), hydroxyzine, and cyproheptadine; and bronchodilators, such as the beta-adrenergic receptor agonists, albuterol (e.g., PROVENTIL®), and terbutaline (BRETHINE®).

Some patients may experience nausea during and after administration of the compounds described herein and/or other therapeutically active agent(s) (e.g., anti-cancer agent(s)). Therefore, anti-emetics can be used in combination with compounds of the present disclosure and/or other therapeutic agent(s) (e.g., anti-cancer agent(s)) to prevent nausea (upper stomach) and vomiting. Suitable anti-emetics include aprepitant (EMEND®), ondansetron (ZOFRAN®), granisetron HCl (KYTRIL®), lorazepam (ATIVAN®, dexamethasone (DECADRON®), prochlorperazine (COMPAZINE®), casopitant (REZONIC® and ZUNRISA®), and combinations thereof.

Medication to alleviate the pain experienced during the treatment period is often prescribed to make the patient more comfortable. Common over-the-counter analgesics, such TYLENOL®, can also be used in combination with compounds of the present disclosure and/or other therapeutically active agent(s) (e.g., anti-cancer agent(s)). Opioid analgesic drugs such as hydrocodone/paracetamol or hydrocodone/acetaminophen (e.g., VICODIN®), morphine (e.g., ASTRAMORPH® or AVINZA®), oxycodone (e.g., OXYCONTIN® or PERCOCET®), oxymorphone hydrochloride (OPANA®), and fentanyl (e.g., DURAGESIC®) can be useful for moderate or severe pain, and can be used in combination with compounds of the present disclosure and/or other therapeutically agent(s) (e.g., anti-cancer agent(s)).

Immunomodulators (e.g., immunooncology agents) of particular interest for use in combination with compounds of the present disclosure include: afutuzumab (available from ROCHE®); pegfilgrastim (NEULASTA®); lenalidomide (CC-5013, REVLIMID®); thalidomide (THALOMID®); actimid (CC4047); and IRX-2 (mixture of human cytokines including interleukin 1, interleukin 2, and interferon 7, CAS 951209-71-5, available from IRX Therapeutics).

Chimeric antigen receptor T-cell (CAR-T) therapies of particular interest for use in combination with compounds of the present disclosure include: tisagenlecleucel (Novartis), axicabtagene ciloleucel (Kite), and tocilizumab (atlizumab; Roche).

Immune checkpoint inhibitors of interest for use in combination with compounds of the present disclosure include: PD-1 inhibitors, such as pembrolizumab (also known as Lambrolizumab, MK-3475, MK03475, SCH-900475, or KEYTRUDA®) and other anti-PD-1 antibodies (as disclosed in Hamid, O. et al. (2013) New England Journal of Medicine 369 (2): 134-44, U.S. Pat. No. 8,354,509, and WO 2009/114335, incorporated by reference in their entirety), nivolumab (also known as MDX-1106, MDX-1106-04, ONO-4538, BMS-936558, or OPDIVO®) and other anti-PD-1 antibodies (as disclosed in U.S. Pat. No. 8,008,449 and WO 2006/121168, incorporated by reference in their entirety), cemiplimab (LIBTAYO®), spartalizumab (PDR001), pidilizumab (CureTech), MEDI0680 (Medimmune), cemiplimab (REGN2810), dostarlimab (TSR-042), PF-06801591 (Pfizer), sinitilimab, toripalimab, tislelizumab (BGB-A317), camrelizumab (INCSHR1210, SHR-1210), AMP-224 (Amplimmune), CBT-501 (CBT Pharmaceuticals), CBT-502 (CBT Pharmaceuticals), JS001 (Junshi Biosciences), IBI308 (Innovent Biologics), INCSHR1210 (Incyte), also known as SHR-1210 (Hengrui Medicine), BGBA317 (Beigene), BGB-108 (Beigene), BAT-I306 (Bio-Thera Solutions), GLS-010 (Gloria Pharmaceuticals; WuXi Biologics), AK103, AK104, AK105 (Akesio Biopharma; Hangzhou Hansi Biologics; Hanzhong Biologics), LZM009 (Livzon), HLX-10 (Henlius Biotech), MEDIO680 (Medimmune), PDF001 (Novartis), PF-06801591 (Pfizer), Pidilizumab (CureTech) also known as CT-011 and other anti-PD-1 antibodies (as disclosed in Rosenblatt, J. et al. (2011) J Immunotherapy 34(5): 409-18, U.S. Pat. Nos. 7,695,715, 7,332,582, and 8,686,119, incorporated by reference in their entirety), REGN2810 (Regeneron), TSR-042 (Tesaro) also known as ANBO11, or CS1003 (CStone Pharmaceuticals). MEDI0680 (Medimmune), is also known as AMP-514. MEDI0680 and other anti-PD-1 antibodies are disclosed in U.S. Pat. No. 9,205,148 and WO 2012/145493, incorporated by reference in their entirety. Further known anti-PD-1 antibody molecules include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entirety. In one embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety. In one embodiment, the anti-PD-1 antibody molecule comprises the CDRs, variable regions, heavy chains and/or light chains of BAP049-Clone-E or BAP049-Clone-B disclosed in US 2015/0210769. The antibody molecules described herein can be made by vectors, host cells, and methods described in US 2015/0210769, incorporated by reference in its entirety. In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entirety).

Immune checkpoint inhibitors of interest for use in combination with compounds of the present disclosure also include: PD-L1 inhibitors, such as atezolizumab (also known as MPDL3280A, RG7446, RO5541267, YW243.55.S70, or TECENTRIQ®) and other anti-PD-L1 antibodies as disclosed in U.S. Pat. No. 8,217,149, incorporated by reference in its entirety, avelumab (BAVENCIO® also known as MSB0010718C) and other anti-PD-L1 antibodies as disclosed in WO 2013/079174, incorporated by reference in its entirety, durvalumab (IMFINZI® or MEDI4736) and other anti-PD-L1 antibodies as disclosed in U.S. Pat. No. 8,779,108, incorporated by reference in its entirety), FAZ053 (Novartis), and BMS-936559 (Bristol-Myers Squibb). In certain embodiments, the PD-L1 inhibitor is KN035 (Alphamab; 3DMed; Ascletis Pharma), Envafolimab (TRACON Pharmaceuticals), BMS 936559 (Bristol-Myers Squibb), CS1001 (CStone Pharmaceuticals, Ligand Pharmaceuticals), CX-072 (CytomX Therapeutics), FAZ053 (Novartis), SHR-1316 (Hengrui Medicine), TQB2450 (Chiatai Tianqing), STI-A1014 (Zhaoke Pharm; Lee's Pharm, Lonza, Sorrento Therapeutics, NantWorks), LYN00102 (Lynkcell), A167 (Harbour BioMed, Kelun Group), BGB-A333 (Beigene), MSB2311 (Mabspace Biosciences), or HLX-20 (Henlius Biotech). In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entirety. In certain embodiments, the PD-L1 inhibitor is Cosibelimab (Fortress Biotech), LY3300054 or Iodapolimab (Eli Lilly), GS-4224 (Gilead Sciences), STI-A1015 (Yuhan, Sorrento Therapeutics), BCD-135 (BIOCAD), Cosibelimab (Dana-Farber Cancer Institute, TG Therapeutics), APL-502 (Apollomics), AK106 (Akeso Biopharma), MSB2311 (Transcenta Holding), TG-1501 (TG Therapeutics), FAZ053 (Novartis). In certain embodiments, the PD-L1 inhibitor is MT-6035 (Molecular Templates), Icaritin and ZKAB001 (Lonza, Lee's Pharmaceutical Holdings, Sorrento Therapeutics, Shenogen Pharma Group), TRIDENT Antibody (MacroGenics, Zai Lab), YBL-007 (Anh-Gook Pharmaceutical, Y-Biologics), HTI-1316 (Hengrui Therapeutics), PD-L1 Oncology Project (Weizmann Institute of Sciences), JS003 (Shanghai Junshi Biosciences), ND021 (Numab Therapeutics, CStone Pharmaceuticals), Toca 521 (Tocagen), STTO1 (STCube). In certain embodiments, the PD-L1 inhibitor is DB004 (DotBio), MT-5050 (Molecular Templates), KD036 (Kadmon). In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety. In one embodiment, the anti-PD-L1 antibody molecule comprises the CDRs, variable regions, heavy chains and/or light chains of BAP058-Clone O or BAP058-Clone N disclosed in US 2016/0108123.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entirety.

In some embodiments, the immune checkpoint inhibitor is a cytotoxic T-lymphocyte-associated modulator. In some embodiments, the immune checkpoint inhibitor aredrugs that target CTLA-4, such as ipilimumab (YERVOY®), tremelimumab, ALPN-202 (Alpine Immune Sciences), RP2 (Replimune), BMS-986249 (Bristol-Myers Squibb), BMS-986218 (Bristol-Myers Squibb), zalifrelimab (Agenus, Ludwig Institute for Cancer Research, UroGen Pharma, Recepta Biopharma), BCD-217 (BIOCAD), Onc-392 (Pfizer, OncoImmune), IBI310 (Innovent Biologics), KN046 (Alphamab), MK-1308 (Merck & Co), REGN4659 (Regeneron Pharmaceuticals), XmAb20717 (Xencor), XmAb22841 (Xencor), Anti-CTLA-4 NF (Bristol-Myers Squibb), MEDI5752 (AstraZeneca), AGEN1181 (Agenus), MGD019 (MacroGenics), ATOR-1015 (Alligator Bioscience), BCD-145 (BIOCAD), PSB205 (Sound Biologics), CS1002 (CStone Pharmaceuticals), ADU-1604 (Aduro Biotech), PF-06753512 (Pfizer), BioInvent-Transgene Research Program (Transgene), AGEN2041 (Agenus, Recepta Biopharam), ATOR-1144 (Alligator Bioscience), CTLA-4 Research Project (Sorrento Therapeutics), PD-L1/CTLA-4 Research Project (Sorrento Therapeutics), HLX13 (Shanghai Henlius Biotech), ISA203 (ISA Pharmaceuticals), PRS-300 Series A (Pieris Pharmaceuticals), BA3071 (BioAtla), CTLA4 Cancer Research Program (Biosortia Pharmaceuticals), RP3 (Replimune), CG0161 (Cold Genesys), APL-509 (Apollomics, JSR), AGEN2041 (Ludwig Institute for Cancer Research), APC 101 (Advanced Proteome), CTLA-4 Inhibitor (Advanced Proteome), BA3071 (BeiGene), BPI-002 (BeyondSpring Pharmaceuticals), CTLA-4 Antibody (Tikcro Technologies), Immuno-Oncology Research Program II (OliPass), PBP1701 (Prestige BioPharma), DB002 (DotBio), DB003 (DotBio), OR-2299 (OncoResponse), NK044 (Alphamab). In certain embodiments, the CTLA-4 inhibitor is ipilimumab. In other embodiments, the CTLA4 inhibitor is tremelimumab.

Immune checkpoint inhibitors of interest for use in combination with compounds of the present disclosure also include: LAG-3 inhibitors. In some embodiments, the LAG-3 inhibitor is chosen from LAG525 (Novartis), BMS-986016 (Bristol-Myers Squibb), or TSR-033 (Tesaro). In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety. In one embodiment, the anti-LAG-3 antibody molecule comprises the CDRs, variable regions, heavy chains and/or light chains of BAP050-Clone I or BAP050-Clone J disclosed in US 2015/0259420. In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entirety. In one embodiment, the anti-LAG-3 antibody molecule is TSR-033 (Tesaro). In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entirety. In one embodiment, the anti-LAG-3 antibody molecule is IMP761 (Prima BioMed). Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entirety. In one embodiment, the anti-LAG-3 inhibitor is a soluble LAG-3 protein, e.g., IMP321 (Prima BioMed), e.g., as disclosed in WO 2009/044273, incorporated by reference in its entirety.

Immune checkpoint inhibitors of interest for use in combination with compounds of the present disclosure also include: Tim-3 inhibitors. In some embodiments, the TIM-3 inhibitor is MGB453 (Novartis) or TSR-022 (Tesaro). In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety. In one embodiment, the anti-TIM-3 antibody molecule comprises the CDRs, variable regions, heavy chains and/or light chains of ABTIM3-huml 1 or ABTIM3-hum03 disclosed in US 2015/0218274. In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121. APE5137, APE5121, and other anti-TIM-3 antibodies are disclosed in WO 2016/161270, incorporated by reference in its entirety. In one embodiment, the anti-TIM-3 antibody molecule is the antibody clone F38-2E2. Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entirety.

In some embodiments, a compound of the present disclosure (e.g., a compound of structural formula I, or a pharmaceutically acceptable salt thereof, or a hydrate of the foregoing) is administered in combination with a checkpoint inhibitor described herein, e.g., to treat pancreatic cancer (e.g., pancreatic ductal adenocarcinoma). In some embodiments, a compound of the present disclosure (e.g., a compound of structural formula I, or a pharmaceutically acceptable salt thereof, or a hydrate of the foregoing) is administered in combination with a checkpoint inhibitor described herein and/or (e.g., or) an agent selected from gemcitabine, nabpaclitaxel, erlotinib, fluorouracil or FOLFIRINOX (a chemotherapy regimen made up of folinic acid, fluorouracil, irinotecan and oxaliplatin), or any combination of two or more of the foregoing, e.g., to treat pancreatic cancer (e.g., advanced pancreatic cancer, pancreatic ductal adenocarcinoma).

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy in combination with compounds of the present disclosure. Suitable cytoprotective agents include amifostine (ETHYOL®), glutamine, dimesna (TAVOCEPT®), mesna (MESNEX®), dexrazoxane (ZINECARD® or TOTECT®), xaliproden (XAPRILA®), and leucovorin (also known as calcium leucovorin, citrovorum factor and folinic acid).

The structure of the active compounds identified by code numbers, generic or trade names may be taken from the actual edition of the standard compendium “The Merck Index” or from databases, e.g., Patents International (e.g., IMS World Publications).

In another aspect of the present disclosure, a kit comprising two or more separate pharmaceutical compositions, at least one of which contains a compound of the present disclosure is provided. In one embodiment, the kit comprises means for separately retaining said compositions, such as a container, divided bottle, or divided foil packet. An example of such a kit is a blister pack, as typically used for the packaging of tablets, capsules and the like.

The kit of the present disclosure may be used for administering different dosage forms, for example, oral and parenteral, for administering the separate compositions at different dosage intervals, or for titrating the separate compositions against one another. To assist compliance, the kit of the present disclosure typically comprises directions for administration.

A compound of the present disclosure may also be used to advantage in combination with known therapeutic processes, for example, the administration of hormones or especially radiation. A compound of the present disclosure may in particular be used as a radiosensitizer, especially for the treatment of tumors which exhibit poor sensitivity to radiotherapy.

In the combination therapies of the present disclosure, the compound of the present disclosure and the other therapeutically active agent may be manufactured and/or formulated by the same or different manufacturers. Moreover, the compound of the present disclosure and the other therapeutically active agent may be brought together into a combination therapy: (i) prior to release of the combination product to physicians (e.g., in the case of a kit comprising the compound of the present disclosure and the other therapeutically active agent); (ii) by the physician (or under the guidance of a physician) shortly before administration; (iii) in the patient themselves, e.g., during sequential administration of the compound of the present disclosure and the other therapeutically active agent.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition or combination of the present disclosure can be in a unit dosage containing from about 1 to about 1000 mg of active ingredient(s) for a subject of from about 50 to about 70 kg, or from about 1 to about 500 mg, from about 1 to about 250 mg, from about 1 to about 150 mg, from about 0.5 to about 100 mg, or from about 1 to about 50 mg of active ingredient(s) for a subject of from about 50 to about 70 kg. The therapeutically effective dosage of a compound, pharmaceutical composition or pharmaceutical combination is dependent, for example, on the species of the subject, the body weight, age and individual condition of the subject, and the disease, disorder or condition or the severity thereof being treated. A physician, clinician or veterinarian of ordinary skill can readily determine the therapeutically effective amount of each of the active ingredients necessary to prevent or treat the progress of the disease, disorder or condition.

The above-cited dosage properties may be demonstrable in in vitro and in vivo tests using advantageously mammals, e.g., mice, rats, dogs, monkeys, or isolated organs, tissues and preparations thereof. The compounds of the present disclosure can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, among other things, between about 0.1 mg/kg to about 500 mg/kg, or between about 1 mg/kg to about 100 mg/kg.

In some embodiments, the concentration of one or more therapeutic agents provided in a pharmaceutical composition is less than 100%, 90%, 80%, 70%, 60%, 50%, 40%, 30%, 20%, 19%, 18%, 17%, 16%, 15%, 14%, 13%, %12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.00010% w/w, w/v or v/v.

In some embodiments, the concentration of one or more therapeutic agents provided in a pharmaceutical composition is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%1, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more therapeutic agents provided in a pharmaceutical composition is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v.

In some embodiments, the concentration of one or more therapeutic agents provided in a pharmaceutical composition is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v.

### Methods of Treatment

It has now been found that the compounds of the present disclosure inhibit TNK1 activity. TNK1 is non-receptor tyrosine kinase. Recently, it was reported that MARK-mediated phosphorylation on TNK1 at S502 promotes an interaction between TNK1 and 14-3-3, which sequesters and inhibits TNK1 kinase activity. Chan, T.-Y., et al., Nature Comm. (2021) 12:5337, the entire contents of which are incorporated herein by reference. TNK1 activity is restored upon release of TNK1 from 14-3-3 and accumulation at ubiquitin clusters. Chan, T.-Y., et al., Nature Comm. (2021) 12:5337. TNK1 has a high affinity for poly-ubiquitin due to a ubiquitin-association domain on its C-terminus. Chan, T.-Y., et al., Nature Comm. (2021) 12:5337.

Accordingly, provided herein are methods of modulating (e.g., inhibiting) TNK1 activity in a cell (e.g., a cell expressing TNK1), comprising contacting the cell with a compound of the present disclosure (e.g., a compound of structural formula I, or a pharmaceutically acceptable salt thereof, or a hydrate of the foregoing), such as a therapeutically effective amount of a compound of the present disclosure. In some embodiments, the cell is in a subject, such as a human. In some embodiments, the TNK1 carries a genetic alteration (e.g., a C-terminal truncation), resulting from a truncating mutation or chromosome rearrangement (e.g., as described in Gu et al.).

Also provided herein are methods of inhibiting TNK1 activity in a subject in need thereof, comprising administering to the subject a therapeutically effective amount of a compound of the present disclosure (e.g., a compound of structural formula I, or a pharmaceutically acceptable salt thereof, or a hydrate of the foregoing). In some embodiments, the TNK1 carries a mutation (e.g., a C-terminal mutation), such as a truncating mutation (e.g., as described in Gu et al.).

Also provided herein are methods of inhibiting TNK1-dependent STAT (e.g., STAT3, STAT5) phosphorylation in a cell (e.g., a cell expressing STAT), comprising contacting the cell with a compound of the present disclosure (e.g., a compound of structural formula I, or a pharmaceutically acceptable salt thereof, or a hydrate of the foregoing), such as a therapeutically effective amount of a compound of the present disclosure. In some embodiments, the cell is in a subject, such as a human. In some embodiments, the TNK1 carries a mutation (e.g., a C-terminal mutation), such as a truncating mutation (e.g., as described in Gu et al.).

Also provided herein are methods of inhibiting TNK1-dependent STAT (e.g., STAT3, STAT5) phosphorylation in a subject in need thereof, comprising administering to the subject a therapeutically effective amount of a compound of the present disclosure (e.g., a compound of structural formula I, or a pharmaceutically acceptable salt thereof, or a hydrate of the foregoing). In some embodiments, the TNK1 carries a mutation (e.g., a C-terminal mutation), such as a truncating mutation (e.g., as described in Gu et al.).

Also provided herein are methods of treating a TNK1-mediated disease, disorder or condition (e.g., cancer, a gastrointestinal disorder, an inflammatory disorder, tissue injury, MODS, sepsis, an autoimmune disorder, a disease, disorder or condition of the microbiome or a disease, disorder or condition resulting from a trauma and/or intestinal injury) in a subject in need thereof, comprising administering to the subject a therapeutically effective amount of a compound of the present disclosure (e.g., a compound of structural formula I, or a pharmaceutically acceptable salt thereof, or a hydrate of the foregoing).

Also provided herein are methods of treating a TNK1-dependent disease, disorder or condition in a subject in need thereof, comprising administering to the subject a therapeutically effective amount of a compound of the present disclosure (e.g., a compound of structural formula I, or a pharmaceutically acceptable salt thereof, or a hydrate of the foregoing). In some embodiments, the TNK1-dependent disease, disorder or condition is a cancer, a gastrointestinal disorder, an inflammatory disorder, tissue injury, MODS, sepsis, an autoimmune disorder, a disease, disorder or condition of the microbiome or a disease, disorder or condition resulting from a trauma or intestinal injury. In some embodiments, the TNK1-dependent disease, disorder or condition is Hodgkin's lymphoma, ALL or AML. ALL and AML have been identified as TNK1-dependent. Chan, T.-Y., et al., Nature Comm. (2021) 12:5337, the entire contents of which are incorporated herein by reference.

Also provided herein are methods of treating sepsis, bacteremia, acute kidney injury, septic shock and/or organ failure (e.g., multi-organ failure) in a subject in need thereof, comprising administering to the subject a therapeutically effective amount of a compound of the present disclosure (e.g., a compound of structural formula I, or a pharmaceutically acceptable salt thereof, or a hydrate of the foregoing).

Also provided herein are methods of improving intestinal barrier function and/or decreasing intestinal permeability and/or regulating intestinal homeostasis in a subject in need thereof, comprising administering to the subject a therapeutically effective amount of a compound of the present disclosure (e.g., a compound of structural formula I, or a pharmaceutically acceptable salt thereof, or a hydrate of the foregoing). Improving intestinal barrier function and/or decreasing intestinal permeability and/or regulating intestinal homeostasis can be a challenge in cancer (e.g., colon cancer), gastrointestinal disorders, inflammatory disorders, tissue injury, MODS, sepsis (e.g., gut-originated sepsis), autoimmune disorders, microbiome health and sensitivity to immunooncology agents, and following a trauma (e.g., severe trauma, hemorrhagic trauma) and/or intestinal injury, where the intestinal barrier can show signs of being damaged or dysregulated. Accordingly, also provided herein are methods of treating a disease, disorder or condition in a subject that would benefit from improved intestinal barrier function and/or decreased intestinal permeability and/or regulated intestinal homeostasis, e.g., a subject having cancer (e.g., a subject having cancer treatable with an immunooncology agent, a subject having cancer and being administered an immunooncology agent), a gastrointestinal disorder, an inflammatory disorder, tissue injury, MODS, sepsis (e.g., gut-originated sepsis), an autoimmune disorder, a disease, disorder or condition of the microbiome (e.g., a dysregulated or unhealthy microbiome) or a disease, disorder or condition resulting from a trauma (e.g., severe trauma, hemorrhagic trauma) and/or intestinal injury, comprising administering to the subject a therapeutically effective amount of a compound of the present disclosure (e.g., a compound of structural formula I, or a pharmaceutically acceptable salt thereof, or a hydrate of the foregoing). Also provided herein are methods of treating a subject following a trauma (e.g., severe trauma, hemorrhagic trauma) and/or intestinal injury, comprising administering to the subject a therapeutically effective amount of a compound of the present disclosure (e.g., a compound of structural formula I, or a pharmaceutically acceptable salt thereof, or a hydrate of the foregoing).

Also provided herein are methods of treating splenomegaly or a disease that can cause splenomegaly, such as cirrhosis, malaria, a viral, bacterial or parasitic infection or sickle cell disease, in a subject in need thereof, comprising administering to the subject a therapeutically effective amount of a compound of the present disclosure (e.g., a compound of structural formula I, or a pharmaceutically acceptable salt thereof, or a hydrate of the foregoing). Although splenomegaly may cause no symptoms in some cases, it can cause one or more of the following symptoms: pain or fullness in the left upper abdomen that may spread to the left shoulder; feeling full without eating or after eating only a small amount; anemia; fatigue; frequent infections; and easy bleeding. Accordingly, also provided herein are methods of treating pain, fullness, anemia, fatigue, infection, and/or bleeding associated with splenomegaly, comprising administering to the subject a therapeutically effective amount of a compound of the present disclosure (e.g., a compound of structural formula I, or a pharmaceutically acceptable salt thereof, or a hydrate of the foregoing).

Also provided herein are methods of treating an inflammatory disorder or reducing inflammation in a subject in need thereof, comprising administering to the subject a therapeutically effective amount of a compound of the present disclosure (e.g., a compound of structural formula I, or a pharmaceutically acceptable salt thereof, or a hydrate of the foregoing).

Also provided herein are methods of treating an infection (e.g., viral infection, bacterial infection, parasitic infection) in a subject in need thereof, comprising administering to the subject a therapeutically effective amount of a compound of the present disclosure (e.g., a compound of structural formula I, or a pharmaceutically acceptable salt thereof, or a hydrate of the foregoing).

Also provided herein are methods of treating tissue injury in a subject in need thereof, comprising administering to the subject a therapeutically effective amount of a compound of the present disclosure (e.g., a compound of structural formula I, or a pharmaceutically acceptable salt thereof, or a hydrate of the foregoing).

Examples of gastrointestinal disorders amenable to the methods disclosed herein include multiple intestinal neoplasia, ischemia/reperfusion injury, colitis (e.g., ulcerative colitis), infectious diarrhea, celiac disease, familial adenomatous polyposis and inflammatory bowel disease (IBD) (e.g., chronic IBD, Crohn's disease, ulcerative colitis).

Examples of inflammatory disorders amenable to the methods disclosed herein include chronic obstructive pulmonary disease (COPD), allergies, cardiovascular disease, hepatitis, asthma, systemic inflammatory response syndrome (SIRS), multiple sclerosis, Goodpasture syndrome, psoriasis, ankylosing spondylitis, antiphospholipid antibody syndrome, gout, arthritis, myositis, scleroderma, Sjogren's syndrome, systemic lupus erythematosus and vasculitis.

Examples of tissue injury amenable to the methods disclosed herein include tissue injury induced by trauma, hemorrhagic shock and physical, chemical and polytrauma.

Examples of autoimmune disorders amenable to the methods disclosed herein include fibrosis, rheumatoid arthritis, multiple sclerosis, systemic lupus erythematosus, type 1 diabetes mellitus, Guillain-Barre syndrome, chronic inflammatory demyelinating polyneuropathy, Graves' disease, Hashimoto's thyroiditis, myasthenia gravis, IBD (e.g., chronic IBD, Crohn's disease, ulcerative colitis), polymyositis, dermatomyositis, inflammatory myositis, ankylosing spondolytis, ulcerative colitis, psoriasis, vasculitis, Sjogren's disease and transplant rejection.

Also provided herein are methods of treating a cancer in a subject in need thereof, comprising administering to the subject a therapeutically effective amount of a compound of the present disclosure (e.g., a compound of structural formula I, or a pharmaceutically acceptable salt thereof, or a hydrate of the foregoing).

A wide variety of cancers, including solid tumors, leukemias, lymphomas, and myelomas are amenable to the methods disclosed herein. In some embodiments, the cancer is a solid tumor cancer. In some embodiments, the cancer comprises a solid tumor (e.g., a colorectal, breast, prostate, lung, pancreatic, renal or ovarian tumor). Accordingly, in some embodiments, the cancer is a solid tumor cancer. In some embodiments, the cancer is selected from one or more of a cancer of the pulmonary system, a brain cancer, a cancer of the gastrointestinal tract, a skin cancer, a genitourinary cancer, head and neck cancer, a sarcoma, a carcinoma, and a neuroendocrine cancer. In various embodiments, the solid tumor cancer is breast cancer, bladder cancer, endometrial cancer, esophageal cancer, liver cancer, pancreatic cancer, lung cancer, cervical cancer, colon cancer, colorectal cancer, gastric cancer, kidney cancer, ovarian cancer, prostate cancer, testicular cancer, uterine cancer, a viral-induced cancer, melanoma or sarcoma. In some embodiments, the cancer is bladder cancer. In some embodiments, the cancer is lung cancer (e.g., non-small cell lung cancer). In other embodiments, the cancer is liver cancer. In some embodiments, the cancer is a sarcoma, bladder cancer or renal cancer. In some embodiments, the cancer is prostate cancer (e.g., castration-resistant prostate cancer, castration-sensitive prostate cancer). In other embodiments, the cancer is bladder cancer, pancreatic cancer, colorectal cancer, glioblastoma, kidney cancer, non-small cell lung carcinoma, prostate cancer, sarcoma, skin cancer, thyroid cancer, testicular cancer or vulvar cancer. In some embodiments, the cancer is endometrial cancer, pancreatic cancer, testicular cancer, renal cancer, melanoma, colorectal cancer, thyroid cancer, bladder cancer, pancreatic cancer, vulvar cancer, sarcoma, prostate cancer, lung cancer or anal cancer. In some embodiments, the cancer is a sarcoma. In some embodiments, the cancer is a renal cell carcinoma.

In some embodiments, the cancer is a non-solid tumor cancer. In some embodiments, the cancer is a hematologic cancer. Hematologic cancers that can be treated according to the methods described herein include leukemias (e.g., acute leukemias, chronic leukemias), lymphomas (e.g., B-cell lymphoma, T-cell lymphoma) and multiple myeloma. In some embodiments, the hematologic cancer is selected from multiple myeloma, myelodysplastic syndrome (MDS), acute myeloid leukemia (AML), acute lymphoblastic leukemia (ALL), acute lymphocytic leukemia, lymphocytic lymphoma, mycosis fungoides, chronic lymphogenous leukemia, chronic lymphocytic leukemia (CLL), mantle cell lymphoma, diffuse large B-cell lymphoma, follicular lymphoma, Hodgkin's lymphoma, non-Hodgkin's lymphoma or myelofibrosis.

In some embodiments provided herein, the hematologic cancer is a leukemia, such as a mutant leukemia (e.g., a mutant AML, a mutant JMML). Mutant leukemias, such as PTPN11/SHP2 mutant (E76K, D61V, and D61Y) leukemias have been implicated in at least AML and juvenile myelomonocytic leukemia (JMML). Jenkins, C., et al., Sci. Signal. 11(539); doi:10.1126/scisignal.aao5617. Jenkins et al. report that TNK2 directly interacts with PTPN11, and that PTPN11-mutant JMML and AML cells are sensitive to TNK2 inhibition.

PTPN11/SHP2 mutations have also been observed in solid tumors. See Jenkins et al. In some embodiments, the cancer comprises a PTPN11/SHP2 mutant (E76K, D61V, and D61Y) solid tumor (e.g., of the breast, lung, prostate, gastrointestinal tract, kidney).

Also provided herein are methods of inhibiting TNK2 activity in a subject in need thereof (e.g., a subject having a mutant leukemia, such as AML or JMML, or a mutant solid tumor, such as a mutant solid tumor of the breast or lung), comprising administering to the subject a therapeutically effective amount of a compound of the present disclosure (e.g., a compound of Formula I, or a subformula thereof, or a pharmaceutically acceptable salt thereof). In some embodiments, the subject has a cancer with a PTPN11/SHP2 mutation (e.g., a mutant leukemia, such as AML or JMML, or a mutant solid tumor, such as a mutant solid tumor of the breast or lung). Also provided herein are methods of inhibiting TNK2 activity in a cell (e.g., a cell from a subject having a mutant leukemia, such as AML or JMML, or a mutant solid tumor, such as a mutant solid tumor of the breast or lung), comprising contacting the cell with a compound of the present disclosure (e.g., a compound of Formula I, or a subformula thereof, or a pharmaceutically acceptable salt thereof). In some embodiments, the TNK2 is expressed in and/or by a PTPN11/SHP2 mutant cell.

In some embodiments, the cancer is a pre-metastatic cancer. In some embodiments, the cancer is a metastatic cancer.

Examples of cancer treatable according to the methods described herein include, but are not limited to, adenocarcinoma of the breast, prostate, and colon; all forms of bronchogenic carcinoma of the lung; myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, lung cancer (e.g., large cell lung cancer, such as squamous cell carcinoma, non-small cell lung), oat cell, papillary, scirrhous, bronchiolar, bronchogenic, squamous cell, and transitional cell). Additional examples of cancer treatable according to the methods described herein include, but are not limited to, histiocytic disorders; leukemia; histiocytosis malignant; Hodgkin's disease; hypereosinophilia, immunoproliferative small; non-Hodgkin's lymphoma; plasmacytoma; reticuloendotheliosis; melanoma; chondroblastoma; chondroma; chondrosarcoma; dermatofibrosarcoma protuberans, fibrotic cancer (myelofibrosis, pancreatic cancer (e.g., pancreatic ductal adenocarcinoma), kidney cancer, liver cancer, lung cancer (e.g., large cell lung cancer, such as squamous cell carcinoma), breast cancer (e.g., inflammatory breast cancer), ovarian cancer (e.g., high grade serious ovarian carcinoma), endometrial cancer, uterine cancer, uterine sarcoma (e.g., uterine leiomyosarcoma), renal cell cancer, sarcoma (e.g., soft tissue sarcoma), malignant fibrous histiocytoma, fibrosarcoma (e.g., dermatofibrosarcoma protuberans) and hepatocellular carcinoma); fibroma; fibrosarcoma; giant cell tumors; histiocytoma; lipoma; liposarcoma; mesothelioma; myxoma; myxosarcoma; osteoma; osteosarcoma; pediatric malignancy, chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatocellular cancer, hepatoma; hidradenoma; islet cell tumor; Leydig cell tumor; papilloma; sertoli cell tumor; theca cell tumor; leiomyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. Yet more examples of cancer treatable according to the methods described herein include, but are not limited to, angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

Further examples of cancers treatable according to the methods described herein include, but are not limited to, Acute Lymphoblastic Leukemia (ALL); Acute Myeloid Leukemia (AML); Adrenocortical Carcinoma; Adrenocortical Carcinoma, Childhood; AIDS-Related Cancer (e.g., Kaposi Sarcoma, AIDS-Related Lymphoma, Primary CNS Lymphoma); Cancer of the anal region; Anal Cancer; Appendix Cancer; Astrocytomas, Childhood; Atypical Teratoid/Rhabdoid Tumor, Childhood, Central Nervous System (CNS); Neoplasms of the CNS (e.g., primary CNS lymphoma, spinal axis tumors, medulloblastoma, brain stem gliomas or pituitary adenomas), Barrett's esophagus (e.g., pre-malignant syndrome), and mycoses fungoides, Basal Cell Carcinoma of the Skin; Bile Duct Cancer; Bladder Cancer; Bladder Cancer, Childhood; Bone Cancer (including Ewing Sarcoma, Osteosarcoma and Malignant Fibrous Histiocytoma); Brain Tumors/Cancer; Breast Cancer; Burkitt Lymphoma; Carcinoid Tumor (Gastrointestinal); Carcinoid Tumor, Childhood; Cardiac (Heart) Tumors, Childhood; Embryonal Tumors, Childhood; Germ Cell Tumor, Childhood; Primary CNS Lymphoma; Cervical Cancer; Childhood Cervical Cancer; Cholangiocarcinoma; Chordoma, Childhood; Chronic Lymphocytic Leukemia (CLL); Chronic Myelogenous Leukemia (CML); Chronic Myeloproliferative Neoplasms; Colorectal Cancer; Childhood Colorectal Cancer; Craniopharyngioma, Childhood; Cutaneous T-Cell Lymphoma (e.g., Mycosis Fungoides and Sezary Syndrome); Ductal Carcinoma In Situ (DCIS); Embryonal Tumors, Central Nervous System, Childhood; Cancer of the Endocrine system (e.g., cancer of the thyroid, pancreas, parathyroid or adrenal glands), Endometrial Cancer (Uterine Cancer); Ependymoma, Childhood; Esophageal Cancer; Childhood Esophageal Cancer; Esthesioneuroblastoma; Ewing Sarcoma; Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST); Childhood Gastrointestinal Stromal Tumors; Germ Cell Tumors; Childhood Central Nervous System Germ Cell Tumors (e.g., Childhood Extracranial Germ Cell Tumors, Extragonadal Germ Cell Tumors, Ovarian Germ Cell Tumors, Testicular Cancer); Gestational Trophoblastic Disease; Gynecologic Tumors ((e.g., uterine sarcomas, carcinoma of the fallopian tubes, carcinoma of the endometrium, carcinoma of the cervix, carcinoma of the vagina or carcinoma of the vulva), Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer; Cutaneous or Intraocular Melanoma; Childhood Intraocular Melanoma; Islet Cell Tumors, Pancreatic Neuroendocrine Tumors; Kaposi Sarcoma; Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Childhood Lung Cancer; Lymphoma; Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma; Mesothelioma, Malignant; Childhood Mesothelioma; Metastatic Cancer; Metastatic Squamous Neck Cancer with Occult Primary; Midline Tract Carcinoma With NUT Gene Changes; Mouth Cancer; Multiple Endocrine Neoplasia Syndromes; Multiple Myeloma/Plasma Cell Neoplasms; Mycosis Fungoides; Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms, Chronic; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip and Oral Cavity Cancer and Oropharyngeal Cancer; Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors; Papillomatosis (Childhood Laryngeal); Paraganglioma; Childhood Paraganglioma; Paranasal Sinus and Nasal Cavity Cancer; Parathyroid Cancer; Penile Cancer; Pharyngeal Cancer; Pheochromocytoma; Childhood Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma; Rhabdomyosarcoma, Childhood; Salivary Gland Cancer; Sarcoma (e.g., Childhood Rhabdomyosarcoma, Childhood Vascular Tumors, Ewing Sarcoma, Kaposi Sarcoma, Osteosarcoma (Bone Cancer), Soft Tissue Sarcoma, Uterine Sarcoma); Sezary Syndrome; Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; Childhood Stomach (Gastric) Cancer; T-Cell Lymphoma, Cutaneous (e.g., Mycosis Fungoides and Sezary Syndrome); Testicular Cancer; Childhood Testicular Cancer; Throat Cancer (e.g., Nasopharyngeal Cancer, Oropharyngeal Cancer, Hypopharyngeal Cancer); Thymoma and Thymic Carcinoma; Thyroid Cancer; Transitional Cell Cancer of the Renal Pelvis and Ureter; Ureter and Renal Pelvis (e.g., renal cell carcinoma, carcinoma of the renal pelvis), benign prostatic hypertrophy, parathyroid cancer, Transitional Cell Cancer; Urethral Cancer; Uterine Cancer, Endometrial; Uterine Sarcoma; Vaginal Cancer; Childhood Vaginal Cancer; Vascular Tumors; Vulvar Cancer; and Wilms Tumor and Other Childhood Kidney Tumors.

Metastases of the aforementioned cancers can also be treated in accordance with the methods described herein.

In some embodiments, the cancer is Hodgkin's lymphoma, pancreatic cancer, B-cell acute lymphoblastic leukemia, multiple myeloma, colorectal cancer, endometrial cancer, lung cancer (e.g., non-small cell lung cancer), bone cancer, medulloblastoma, glioma, kidney cancer, ovarian cancer, breast cancer or astrocytoma.

In some embodiments, the cancer is prostate cancer (e.g., castration-resistant prostate cancer). In some embodiments, the cancer is pancreatic cancer (e.g., pancreatic ductal adenocarcinoma, advanced pancreatic cancer). In some embodiments, the cancer is Hodgkin's lymphoma. In some embodiments, the cancer is colon cancer. In some embodiments, the cancer is colorectal cancer. In some embodiments, the cancer is lung cancer (e.g., non-small cell lung cancer).

Human, full-length TNK1, such as that found in K562 CML cells, is associated with UniProtKB Accession No. Q13470. Mutations (e.g., truncating mutation(s), rearrangement(s), such as inversion(s)) of TNK1 (e.g., C-terminal mutations, such as C-terminal truncating mutations) have also been observed in humans, for example, in the Hodgkin's lymphoma cell line L540. For example, Gu, T.-L., et al., Leukemia (2010), 24, 861-865, the entire contents of which are incorporated by reference herein, disclose a variant of TNK1 in which the 5′ part of TNK1 including the kinase domain is fused to sequences composed of 31 base pairs from 5′ untranslated region, complete exon 2 and the first 52 base pairs of exon 3 of chromosome 17 open reading frame 61 (C17ORF61) gene, resulting from paracentric inversion (17)(p13.1). See, in particular, FIG. 1(c) of Gu et al. The variant of TNK1 disclosed in Gu et al. lacks the C-terminal inhibitory sequences of full-length TNK1. Gu et al. also disclose that phosphorylation of STAT5 is a reliable surrogate marker for tyrosine kinase activity.

Thus, in some embodiments, the cancer is associated with a TNK1 mutation (e.g., a C-terminal mutation), such as a truncating mutation (e.g., as described in Gu et al.). In some embodiments, the cancer is associated with dysregulated (e.g., enhanced, increased) TNK1 phosphorylation. Examples of cancers associated with a TNK1 mutation include Hodgkin's lymphoma, colorectal cancer and lung cancer (e.g., non-small cell lung cancer). Examples of TNK1 mutations in colorectal cancer include, but are not limited to, R458W, R562I and E522fs. An example of a cancer associated with dysregulated (e.g., enhanced, increased) TNK1 phosphorylation is Hodgkin's lymphoma.

In some embodiments, the cancer is associated with TNK1-dependent STAT5 phosphorylation. In some embodiments, the cancer is associated with dysregulated (e.g., enhanced, increased) STAT5 phosphorylation. An example of a cancer associated with TNK1-dependent and/or dysregulated (e.g., enhanced, increased) STAT5 phosphorylation is Hodgkin's lymphoma.

Also provided herein is a method of treating a TNK1-mediated disease, disorder or condition (e.g., a TNK1-mediated disease, disorder or condition described herein) in a subject carrying a TNK1 mutation (e.g., a TNK1 mutation described herein, e.g., a C-terminal mutation, such as a truncating mutation, e.g., as described in Gu et al.), comprising providing a subject determined to carry a TNK1 mutation; and administering to the subject a therapeutically effective amount of a compound of the present disclosure (e.g., a compound of structural formula I, or a pharmaceutically acceptable salt thereof, or a hydrate of the foregoing). In some embodiments, the subject carries a mutation in a TNK1 gene. In some embodiments, the subject carries a mutation in the TNK1 protein, e.g., that results from a mutation in a TNK1 gene.

Also provided herein is a method of treating a TNK1-mediated disease, disorder or condition (e.g., a TNK1-mediated disease, disorder or condition described herein) in a subject carrying a TNK1 mutation (e.g., a TNK1 mutation described herein, e.g., a C-terminal mutation, such as a truncating mutation, e.g., as described in Gu et al.), comprising determining whether the subject carries a TNK1 mutation; and administering to the subject a therapeutically effective amount of a compound of the present disclosure (e.g., a compound of structural formula I, or a pharmaceutically acceptable salt thereof, or a hydrate of the foregoing) if it is determined that the subject carries the TNK1 mutation. In some embodiments, the subject carries a mutation in a TNK1 gene. In some embodiments, the subject carries a mutation in the TNK1 protein, e.g., that results from a mutation in a TNK1 gene.

Also provided herein is a method of mediating apoptosis in a cell, comprising contacting the cell with a compound of the present disclosure (e.g., a compound of structural formula I, or a pharmaceutically acceptable salt thereof, or a hydrate of the foregoing). In some aspects, the cell is in a subject, such as a human.

Also provided herein is a method of reducing inflammation in a cell, comprising contacting the cell with a compound of the present disclosure (e.g., a compound of structural formula I, or a pharmaceutically acceptable salt thereof, or a hydrate of the foregoing). In some aspects, the cell is in a subject, such as a human.

A therapeutically effective amount of a therapeutically active agent (e.g., a compound of the present disclosure) to be administered to a subject in accordance with the methods described herein can be determined by a clinician of ordinary skill using the guidance provided herein and other methods known in the art. For example, suitable dosages may range, depending on the route of administration, among other things, from about 0.1 mg/kg to about 500 mg/kg, or from about 1 mg/kg to about 100 mg/kg. In some embodiments, a suitable dosage of a compound of the present disclosure is from about 1 mg to about 500 mg, e.g., from about 62 mg to about 229 mg, of the compound of structural formula I, or a pharmaceutically acceptable salt thereof, or a hydrate of the foregoing, based on the molecular weight of the compound of structural formula I as a free base.

A compound of the present disclosure can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. The preferred mode of administration can vary depending on the particular compound chosen. In some embodiments, the compound of the present disclosure is administered orally. In some embodiments, the compound of the present disclosure is administered intravenously.

A compound of the present disclosure can also be administered in combination with one or more other therapies (e.g., a chemotherapy, such as a chemotherapeutic agent; an immunotherapy, such as an immunotherapeutic agent, an immunooncology agent). Accordingly, in some embodiments, the methods further comprise administering to the subject a therapeutically effective amount of one or more additional therapies (e.g., therapeutically active agents). Suitable therapies and therapeutically active agents for use in combination with a compound of the present disclosure in the methods disclosed herein include those discussed herein in connection with combination therapy and pharmaceutical combinations.

When administered in combination with another therapy, the compound of the present disclosure can be administered before, after or concurrently with the other therapy (e.g., an additional therapeutically active agent(s)). When two or more therapeutically active agents are co-administered simultaneously (e.g., concurrently), the compound of the present disclosure and additional therapeutically active agent(s) can be in separate formulations or the same formulation. Alternatively, the compound of the present disclosure and other therapy can be administered sequentially (e.g., as separate compositions) within an appropriate time frame as determined by a skilled clinician (e.g., a time sufficient to allow an overlap of the pharmaceutical effects of the compound of the present disclosure and the other therapy).

### EXEMPLIFICATION

### Example 1. Form Selection

A salt screening was conducted on the free base form of the compound of structural formula I to assess whether a salt form would provide benefits over the free base form. The starting material (Lot No: SY18002581-8, ID No: 818718-01-A) was crystalline and initially named Freebase Type A. The Freebase Type A sample was determined to be a mixture of Freebase Type E+F after polymorph screening of freebase was conducted. Starting from this batch of freebase sample (Freebase Type E+F) (818718-01-A), salt screening was performed under 84 conditions using 17 acids (extra molar charge ratios of 2:1 for H₂SO₄ and 2:1, 3:1, 4:1 for HCl) in four solvent systems. From all the screening experiments, a total of 35 crystalline salt hits were isolated and characterized by x-ray powder diffraction (XRPD), thermo-gravimetric analysis (TGA), and differential scanning calorimetry (DSC). The stoichiometric ratio of salt hits was determined by proton nuclear magnetic resonance (H NMR) or high-performance liquid chromatography (HPLC) combined with ion chromatography (IC). Based on the physical properties of the hits from screening, Succinate Type B, L-Malate Type B, Mesylate Type A, Besylate Type A and L-Tartrate Type A were selected as potential candidate salts for scale-up.

The salt leads of L-Malate Type B and Mesylate Type A were successfully re-prepared. The preparation experiments of L-Tartrate Type A and Besylate Type A resulted in L-Tartrate Type A+B mixture and Besylate Type A+B mixture, respectively. The crystallinity of re-prepared Succinate Type B was relatively low, which might affect the water uptake of Succinate Type B (8.22% at 25° C./80% RH by dynamic vapor sorption (DVS)). Combined with reproducibility and crystallinity of the potential candidate salts, L-Malate Type B and Mesylate Type A were selected as leading salts for further evaluation.

L-Malate Type B and Mesylate Type A were evaluated on hygroscopicity, kinetic solubility in bio-relevant buffers (water/SGF/FaSSIF), and solid-state stability at 25° C./60% relative humidity (RH) and 40° C./75% RH (open) for two weeks, using freebase sample (Freebase Type E+F) received as control. Characterization and evaluation data are summarized in Table 1.1. The results showed that both L-Malate Type B and Mesylate Type A were hygroscopic, and Freebase Type E+F (818718-01-A) was slightly hygroscopic. No form change was observed for Freebase Type E+F, L-Malate Type B and Mesylate Type A after DVS. L-Malate Type B and Mesylate Type A showed increased solubility in water at 37° C. using freebase starting material (Freebase Type E+F) as control. Freebase Type E+F, L-Malate Type B and Mesylate Type A showed relatively high solubility in SGF at 37° C. L-Malate Type B showed much higher solubility than Mesylate Type A and Freebase Type E+F in FaSSIF at 37° C. No solid residue was obtained for the two salts in three media (except for Mesylate Type A in FaSSIF), so XRPD test was not performed. A new form with low crystallinity was observed after Mesylate Type A was suspended in FaSSIF at 37° C. for 1 hour. Freebase Type E+F turned to Freebase Type E after kinetic solubility test in all the three media. All of Freebase Type E+F, L-Malate Type B and Mesylate Type A showed good physical and chemical stability as evidenced by no form change or substantial HPLC purity decrease after storage for two weeks.

A new batch of freebase sample (ID No.: 818718-45-A) was used for polymorphism evaluation. Characterization data showed batch 818718-45-A displayed a new XRPD pattern, named as Freebase Type B. Using Freebase Type B as starting material, Mesylate Type A was successfully re-prepared on 5.0 g scale for polymorph screening.

Using Mesylate Type A and Freebase Type B as starting materials, preliminary polymorph screening was conducted under 66 and 67 conditions, respectively, using different methods of slurry conversion, evaporation, liquid vapor diffusion, solid vapor diffusion, slow cooling and anti-solvent addition. For mesylate, no new form except for Mesylate Type A was discovered from polymorph screening. For freebase, anhydrates Freebase Type B/E/F/G, hydrate Freebase Type C and DCM solvate Freebase Type D were discovered. Slurry competition experiments results among Freebase Type B, C, E, F and G showed: a) in TPAc systems from 5° C. to 50° C., and in acetone/H2O with different a_{w} systems at room temperature (RT), Freebase Type E was observed; b) there might be a potential metastable form, such as EtOH or ACN solvate, in ACN, EtOH/H2O (a_{w}=0.2) and EtOH systems, which might turn to Freebase Type C or G quickly.

Table 1.1 summarizes the characterization and evaluation of the salt leads and free base.

TABLE 1.1
Characterization and Evaluation Summary of Salt Leads and Free Base

| | Freebase Type | | |
|---|---|---|---|
| Crystal Form | E + F | L-Malate Type B | Mesylate Type A |
| Sample ID | 818718-01-A | 818718-21-A1 | 818718-21-A5 |
| Weight Loss in | 2.3 | 3.7 | 1.0 |
| TGA (%) | | | |
| Endotherm in DSC | 90.6, 132.0, | 62.5, 168.6 | 70.2, 263.3 |
| (° C., peak) | 151.5, 159.4 | | |
| HPLC Purity (area %) | 98.63 | 98.93 | 99.26 |
| Stoichiometry | Not applicable | 1.17 | 0.97 |
| (acid/FB)^{#} | | | |
| Hygroscopicity (%)* | Slightly | Hygroscopic (3.00) | Hygroscopic (2.74) |
| | hygroscopic (1.55) | | |
| Form Change after DVS | No | No | No |

| | |
|---|---|
| Kinetic Solubility | L-Malate Type B, Mesylate Type A and Freebase Type E + F |
| | showed relatively high solubility in SGF at 37° C. L-Malate |
| | Type B showed much higher solubility than Mesylate Type A |
| | and Freebase Type E + F in FaSSIF at 37° C. L-Malate Type B |
| | and Mesylate Type A showed increased solubility in water at |
| | 37° C. using Freebase Type E + F as control. |
| Stress Stability | No form change or significant HPLC purity decrease was |
| | observed. |

*Based on water uptake up at 25° C./80% RH: very hygroscopic - >15%, hygroscopic - 2~15%, slightly hygroscopic - 0.2~2%, non-hygroscopic - <0.2%;
^{#}determined by NMR.

According to the basic pKa value of 6.88 estimated by Marvin and approximate solubility of freebase starting material (818718-01-A) at room temperature (25±3° C.), 17 acids and four solvent systems were used for the screening. For each solvent of acetone, EtOAc, DCM and THF/H2O (19:1, v/v), free base and corresponding acid were mixed in a molar charge ratio of 1:1 (one extra molar charge ratio of 2:1 for H2SO4 and three extra charge ratios of 2:1, 3:1, 4:1 for HCl), and stirred at RT overnight. The solids obtained were characterized by XRPD, and the clear solutions were transferred to stirring at 5° C. for three days. Gel was transferred to slurry at 50° C. for three days (except for DCM system). The solids obtained were tested by XRPD. For the clear solutions and gel, anti-solvent (n-heptane for acetone, EtOAc and DCM; H2O for THF/H2O) was added. The mixtures were then stirred at 5° C. overnight. The solids were characterized by XRPD, and the clear solutions were transferred to slow evaporation at RT. The clear solutions, obtained from ACN/H2O (19:1, v/v) systems, were transferred to vacuum volatilization at RT. As summarized in Table 1.2, a total of 35 crystalline hits were obtained. All the potential salt hits obtained were isolated, dried at 50° C. for 2-3 hours and then characterized by XRPD, TGA, DSC and HPLC purity, with the stoichiometry determined by NMR or HPLC/IC. The characterization data are summarized in Table 1.3.

TABLE 1.2
Summary of Salt Screening Results

| | | | | | D |
|---|---|---|---|---|---|
| | | A | B | C | ACN/H₂O |
| Acid | Solvent | Acetone | EtOAc | THF | (19:1, v/v) |
| 0 | Blank | Freebase | Freebase | Freebase | Freebase |
| | | Type E^{#} | Type E^{#} | Type F** | Type E^{&} |
| 1 | HCl (1:1) | Mono-HCl | Mono-HCl | Mono-HCl | Mono-HCl salt |
| | | salt Type A | salt Type | salt Type B | Type B |
| | | | A + B | | |
| 2 | HCl (2:1) | Mono-HCl | Mono-HCl | Bis-HCl salt | Bis-HCl salt |
| | | salt Type | salt Type | Type B | Type B |
| | | A + FB Type A | A + B | | |
| 3 | HCl (3:1) | Bis-HCl salt | Tri-HCl salt | Bis-HCl salt | Bis-HCl salt |
| | | Type A | Type A + Bis- | Type B | Type B |
| | | | HCl salt Type B | | |
| 4 | HCl (4:1) | Tri-HCl salt | Tri-HCl salt | Bis-HCl salt | Bis-HCl salt |
| | | Type A | Type B | Type B** | Type B |
| 5 | H₂SO₄ (1:1) | Mono-sulfate | Mono-sulfate | Amorphous^{&} | Mono-sulfate |
| | | Type A | Type A | | Type B* |
| 6 | H₂SO₄ (2:1) | Bis-sulfate | Amorphous | Gel** | Gel^{&&} |
| | | Type A (low | | | |
| | | crystallinity) | | | |
| 7 | H₃PO₄ | Phosphate | Amorphous^{&} | Phosphate | Phosphate |
| | | Type A | | Type B | Type A |
| 8 | Citric acid | Citrate Type A | Citrate Type A | Citrate Type A | Citrate Type A |
| 9 | Maleic acid | Maleate Type A | Maleate Type B | Amorphous^{&} | Maleate Type A |
| | | | | | (low crystallinity)^{&&} |
| 10 | Fumaric acid | Fumarate | Fumarate | Amorphous^{#} | Fumarate |
| | | Type A | Type B | | Type C |
| 11 | Glycolic Acid | Glycolate | Glycolate | Glycolate | Amorphous^{&&} |
| | | Type A | Type B | Type A + B^{#} | |
| 12 | Acetic acid | Freebase | Freebase | Acetate | Gel^{&&} |
| | | Type E^{&} | Type E^{&} | Type A^{#} | |
| 13 | L-Malic acid | L-Malate | L-Malate | L-Malate | Gel^{&&} |
| | | Type A | Type B | Type C^{#} | |
| 14 | L-Tartaric acid | L-Tartrate | L-Tartrate | L-Tartrate | L-Tartrate |
| | | Type A | Type A | Type A | Type B |
| 15 | Succinic acid | Succinate | Succinate | Low | Succinate |
| | | Type A | Type B | crystallinity^{&} | Type C^{&&} |
| 16 | L-Lactic acid | Low | Low | Low | Gel^{&&} |
| | | crystallinity^{&} | crystallinity^{#} | crystallinity^{&} | |
| 17 | Oxalic acid | Oxalate | Oxalate | Oxalate | Low crystallinity |
| | | Type A | Type A | Type A | |
| 18 | Methanesulfonic | Mesylate | Mesylate | Mesylate | Mesylate |
| | acid | Type A | Type A | Type A^{&} | Type A |
| 19 | Benzenesulfonic | Besylate | Besylate | Besylate | Besylate |
| | acid | Type B^{#} | Type A | Type B^{&} | Type B^{#} |
| 20 | p- | Tosylate | Tosylate | Tosylate | Gel^{&&} |
| | Toluenesulfonic | Type B^{&} | Type A | Type B^{&} | |
| | acid | | | | |
| 21 | HBr | HBr salt | HBr salt | HBr salt | HBr salt |
| | | Type A | Type A | Type B^{&} | Type B |

^{#}obtained from slurry at 5° C.;
*obtained from slurry at 50° C.;
^{&}obtained from anti-solvent addition;
**obtained from evaporation at RT;
^{&&}obtained from vacuum volatilization at RT.

TABLE 1.3
Characterization Summary of Crystalline Hits

| | | | | | Weight | | | |
|---|---|---|---|---|---|---|---|---|
| | | Acid | | | Loss in | Endotherm | Stoichio- | |
| | | Safety | | Sample | TGA | in DSC (° C., | metry | Purity |
| Hit | | Class | M_{w} | ID | (%) | peak) | (acid/FB)^{#} | (area %) |
| Mono- | Type | | | 818718- | 6.7 | 93.4, 175.7 | 0.98 | 99.23 |
| | A | | | 03-A1 | | | | |
| HCI Salt | Type | | | 818718- | 8.8 | 101.1, | 0.97 | 99.07 |
| | B | | | 03-D1 | | 199.5 | | |
| Bis-HC1 | Type | | 36.46 | 818718- | 12.8 | 113.5, | 2.13^{&} | 98.54 |
| | A | | | 03-D3 | | 189.6 | | |
| Salt | Type | | | 818718- | 13.1 | 67.3, 88.6, | 2.48** | 98.71 |
| | B | | | 03-A3 | | 97.4, 170.2, | | |
| | | | | | | 186.4 | | |
| Tri-HC1 | Type | | | 818718- | 14.7 | 63.6, 100.8, | 3.04 | 98.47 |
| | A | | | 03-A4 | | 180.9, | | |
| | | | | | | 183.6 | | |
| Salt | Type | | | 818718- | 15.0 | 65.4, 92.5, | 3.16^{&} | 97.98 |
| | B | | | 03-B4 | | 178.5, | | |
| | | | | | | 193.8 | | |
| Mono- | Type | | | 818718- | 5.5 | 83.5, 183.4 | 0.97 | 98.71 |
| | A | | | 03-A5 | | | | |
| sulfate | Type | | 98.08 | 818718- | 5.1 | 176.7 | 1.04 | 98.45 |
| | B | | | 03-D5 | | | | |
| Bis- | Type | | | 818718- | 10.2 | 73.7, 145.7, | 2.02 | 98.56 |
| sulfate | A | | | 03-A6 | | 154.6 | | |
| Phosphate | Type | | 98.00 | 818718- | 8.0 | 93.7, | 0.97 | 98.86 |
| | A | | | 03-C7 | | 113.6, 130.3 | | |
| | Type | | | 818718- | 8.2 | 77.6, 153.9 | 0.93 | 99.27 |
| | B | | | 03-D7 | | | | |
| Maleate | Type | | 116.08 | 818718- | 5.8 | 86.5, 140.2, | 1.15 | 98.80 |
| | A | | | 03-B9 | | 202.6 | | |
| Fumarate | Type | I | 116.08 | 818718- | 5.2 | 72.4, 135.7, | 0.58 | 99.18 |
| | A | | | 03-A10 | | 171.5*, | | |
| | | | | | | 183.1* | | |
| | | | | | | 187.5*, | | |
| | | | | | | 193.4*, | | |
| | | | | | | 228.9 | | |
| | Type | | | 818718- | 5.2 | 61.4, 127.6, | 1.17 | 99.21 |
| | B | | | 03-B10 | | 140.6, | | |
| | | | | | | 178.9 | | |
| | Type | | | 818718- | 6.0 | 50.1, 78.1, | 1.30 | 98.95 |
| | C | | | 03-D10 | | 104.5, | | |
| | | | | | | 142.7 | | |
| Citrate | Type | | 192.14 | 818718- | 4.7 | 51.0, 107.4, | 1.10 | 99.05 |
| | A | | | 03-D8 | | 146.8, | | |
| | | | | | | 159.8 | | |
| | | | | | | 175.0 | | |
| Glycolate | Type | | 76.05 | 818718- | 5.7 | 76.3, 130.8, | 1.69 | 99.22 |
| | A | | | 03-A11 | | 142.7 | | |
| | Type | | | 818718- | 6.0 | 83.3, 142.3 | 1.81 | 98.94 |
| | B | | | 03-B11 | | | | |
| Acetate | Type | | 60.05 | 820105- | 9.2 | 78.3, 125.2, | 1.01 | 98.69 |
| | A | | | 01-C1 | | 144.5 | | |
| L-Malate | Type | | 134.09 | 820105- | 8.3 | 79.9, 135.4, | 1.04 | 99.47 |
| | A | | | 01-A2 | | 192.8, | | |
| | | | | | | 213.3 | | |
| | Type | | | 820105- | 2.0 | 72.2, 168.2 | 1.01 | 98.65 |
| | B | | | 01-B2 | | | | |
| | Type | | | 820105- | 5.5 | 73.7, 141.1, | 1.27 | 98.66 |
| | C | | | 01-C2 | | 161.1, | | |
| | | | | | | 188.5, | | |
| | | | | | | 212.9 | | |
| L-Tartrate | Type | | 150.09 | 820105- | 2.0 | 45.1, 221.0 | 0.98 | 98.82 |
| | A | | | 01-A3 | | | | |
| | Type | | | 820105- | 5.6 | 112.2 | 0.98 | 98.79 |
| | B | | | 01-D3 | | | | |
| Succinate | Type | | 118.09 | 820105- | 2.0 | 61.7, 154.3 | 1.01 | 99.32 |
| | A | | | 01-A4 | | | | |
| | Type | | | 820105- | 1.3 | 65.3, 157.2 | 1.09 | 99.07 |
| | B | | | 01-B4 | | | | |
| | Type | | | 820105- | 14.9 | 68.3, 85.5, | 0.84 | 99.36 |
| | C | | | 01-D4 | | 111.6 | | |
| Oxalate | Type | II | 90.04 | 820105- | 3.8 | 78.3, 149.3 | 0.91 | 99.15 |
| | A | | | 01-A6 | | | | |
| Mesylate | Type | | 96.10 | 820105- | 1.4 | 66.6, 265.9 | 1.04 | 99.37 |
| | A | | | 01-D7 | | | | |
| Besylate | Type | | 158.18 | 820105- | 2.4 | 68.4, 216.7 | 0.94 | 99.24 |
| | A | | | 01-B8 | | | | |
| | Type | | | 820105- | 5.8 | 112.7, | 0.93 | 99.66 |
| | B | | | 01-D8 | | 216.8 | | |
| Tosylate | Type | | 172.21 | 820105- | 2.4 | 70.9, 182.1, | 0.99 | 98.94 |
| | A | | | 01-B9 | | 208.7 | | |
| | Type | | | 820105- | 1.9 | 72.9, 182.5 | 1.04 | 98.80 |
| | B | | | 01-C9 | | | | |
| HBr Salt | Type | III | 90.04 | 820105- | 5.8 | 88.7, 185.6 | 1.07 | 98.84 |
| | A | | | 01-B10 | | | | |
| | Type | | | 820105- | 7.4 | 65.3, 76.1, | 0.98 | 98.64 |
| | B | | | 01-D10 | | 152.1, | | |
| | | | | | | 201.7 | | |

^{#}estimated by IC/HPLC or NMR;
**although the stoichiometry of 818718-03-A3 was 2.48, the sample was named as Bis-HCl salt;
*exotherm;
^{&}since the total recovery was out of range (95%~105%), the stoichiometric ratio may not be accurate and only used for reference.

L-Malate Type B, Succinate Type B and Mesylate Type A were successfully re-prepared to hundreds of milligrams. The detailed preparation procedures are described in Table 1.4, and the characterization data are summarized in Table 1.1. For Besylate Type A and L-Tartrate Type A, the Besylate Type A+B mixture and L-Tartrate Type A+B mixture were obtained in the re-preparation experiments of Besylate Type A and L-Tartrate Type A, respectively. From the perspective of reproducibility, Besylate Type A and L-Tartrate Type A were not chosen for further evaluation.

TABLE 1.4
Preparation Procedures of Salt Leads

Crystal

| Form | Preparation Procedures | |
|---|---|---|
| L-Malate | 1. | Add 304.8 mg freebase (818718-01-A) and 18.0 mL |
| Type B | | EtOAc to a 100 mL glass vial. A clear solution was |
| (818718- | | obtained. |
| 21-A1) | 2. | Add 83.0 mg L-malic acid into the above solution of |
| | | freebase. |
| | 3. | Stir the mixture at RT for five minutes, and a suspension |
| | | was obtained. |
| | 4. | Sample for XRPD after stirring for one day, and the |
| | | pattern confirmed to L-Malate Type B. |
| | 5. | Centrifuge the suspension obtained and dry the wet cake |
| | | at 50° C. After 1.5 hours, the samples were transferred to |
| | | vacuum drying at RT for 0.5 hour. |
| | 6. | Collect solids of 349.5 mg, with a yield of 92.6%. |
| Succinate | 1. | Add 303.2 mg freebase (818718-01-A) and 18.0 mL |
| Type B | | EtOAc to a 100-mL glass vial. A clear solution was |
| (818718- | | obtained. |
| 21-A2) | 2. | Add 66.1 mg succinic acid into the above solution of |
| | | freebase. |
| | 3. | Stir the mixture at RT. |
| | 4. | Sample for XRPD after stirring overnight, and the |
| | | pattern conformed to Succinate Type B. |
| | 5. | Centrifuge the suspension obtained and dry the wet cake |
| | | at 50° C. After 1.5 hours, the samples were transferred to |
| | | vacuum drying at RT for 0.5 hour. |
| | 6. | Collect solids of 258.8 mg, with a yield of 70.3%. |
| Mesylate | 1. | Add 305.2 mg freebase (818718-01-A) and 3.5 mL |
| Type A | | THF/H₂O (19:1, v/v) to a 20-mL glass vial. A clear |
| (818718- | | solution was obtained. |
| 21-A5) | 2. | Add 35.8 μL methanesulfonic acid to 1.5 mL THF/H20 |
| | | (19:1, v/v) in a 3-mL glass vial, and add the solution of |
| | | acid into the solution of freebase. Stir the mixture at RT. |
| | 3. | Sample for XRPD after stirring overnight, and the |
| | | pattern conformed to Mesylate Type A. |
| | 4. | Centrifuge the suspension obtained and dry the wet cake |
| | | at 50° C. After 1.5 hours, the samples were transferred to |
| | | vacuum drying at RT for 0.5 hour. |
| | 5. | Collect solids of 160.3 mg, with a yield of 52.4%. |

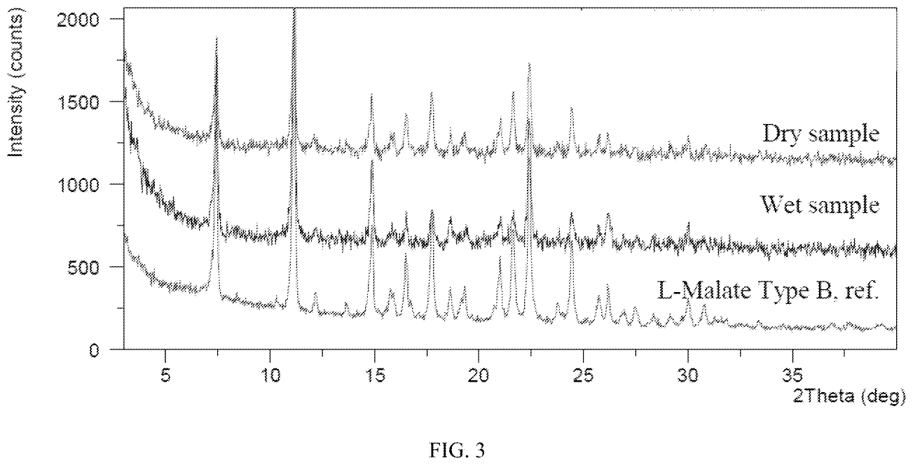

L-Malate Type B (818718-21-A1) was successfully re-prepared as evidenced by XRPD results in FIG. 3. As per TGA and DSC data summarized in Table 1.1, the sample showed a weight loss of 3.7% up to 150° C., and DSC curve showed two thermal signals at 62.5° C. and 168.6° C. (peak temperature; peak onset occurred at 164.1° C. Around 0.08 molar EtOAc (about 1.0 weight percent) was detected by ¹H NMR. Based on the integrals, the stoichiometric ratio of L-malic acid and freebase was determined to be 1.17.

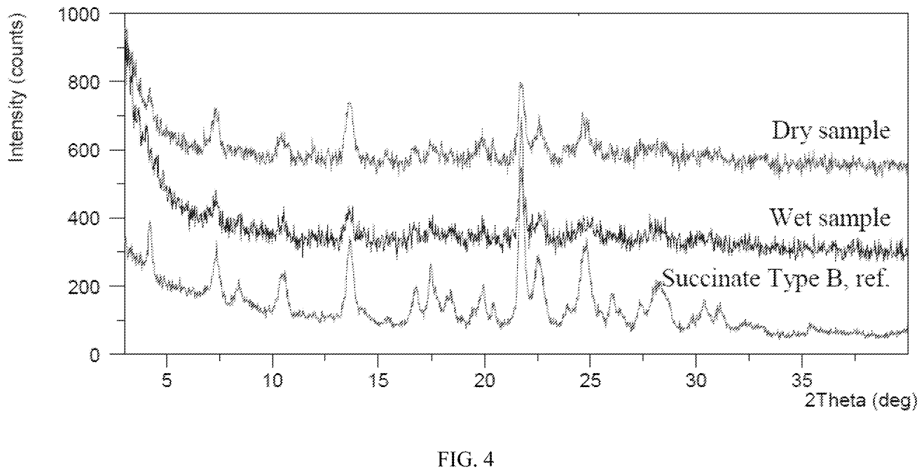

Succinate Type B (818718-21-A2) was successfully re-prepared, as evidenced by XRPD results in FIG. 4. As per TGA and DSC data summarized in Table 1.1, the sample showed a weight loss of 1.8% up to 150° C. and two thermal signals at 53.0° C. and 157.2° C. (peak temperature; peak onset occurred at 151.1° C.). No peak of EtOAc was observed in ¹H NMR. Based on the integrals, the stoichiometric ratio of succinic acid and freebase was determined to be 1.08.

Mesylate Type A (818718-21-A5) was successfully re-prepared, as evidenced by XRPD. As per TGA and DSC data summarized in Table 1.1, the sample showed a weight loss of 1.0% up to 150° C. and two thermal signals at 70.2° C. and 263.3° C. (peak temperature; peak onset occurred at 258.5° C.). No peak of THE was observed in ¹H NMR. Based on the integrals, the stoichiometric ratio of methanesulfonic acid and freebase was determined to be around 0.97.

Further evaluation study on hygroscopicity, kinetic solubility, and solid-state stability was conducted to better understand the physicochemical properties of the candidate salts, using freebase as control.

Hygroscopicity. DVS isotherm plot was collected at 25° C. to investigate the solid form stability as a function of humidity. All of Freebase Type E+F, L-Malate Type B, Succinate Type B and Mesylate Type A were equilibrated at ambient humidity (70% RH or 80% RH) to prevent form change before testing.

As evidenced by the water uptake of 3.00% of L-Malate Type B (818718-21-A1), 8.22% of Succinate Type B (818718-21-A2) and 2.74% of Mesylate Type A (818718-21-A5) up to 80% RH, all of L-Malate Type B, Succinate Type B and Mesylate Type A were hygroscopic. Freebase Type E+F (818718-01-A) was slightly hygroscopic with a water uptake of 1.55% at 80% RH (FIG. 3-7). No form change was observed for all of L-Malate Type B, Succinate Type B, Mesylate Type A and Freebase Type E+F before and after DVS test. It is noteworthy that when the humidity was greater than 50% RH, a significantly increased mass was observed for Succinate Type B (818718-21-A2). The re-prepared Succinate Type B showed low crystallinity, which might affect the water uptake of Succinate Type B. Considering the relatively low crystallinity and hygroscopicity of Succinate Type B, further study was not performed on Succinate Type B.

Kinetic Solubility. Kinetic solubility of salt leads was measured in water, SGF and FaSSIF to evaluate their solubility and disproportionation risk, using Freebase Type E+F (818718-01-A) as control. All the solubility samples (initial solid loading of about 10 mg/mL) were kept rolling on a rolling incubator at a speed of 25 rpm, and sampled at 1, 4 and 24 hours at 37° C. After centrifugation, supernatants were collected for HPLC and pH tests, and wet cakes were collected for XRPD characterization.

The results are summarized in Table 1.5. L-Malate Type B and Mesylate Type A showed increased solubility in water at 37° C. using Freebase Type E+F as control. L-Malate Type B, Mesylate Type A and Freebase Type E+F showed relatively high solubility in SGF at 37° C. L-Malate Type B showed much higher solubility than Mesylate Type A and Freebase Type E+F in FaSSIF at 37° C. After L-Malate Type B was suspended for 1, 4 and 24 hours in three media, clear solutions were obtained. For Mesylate Type A, clear solutions were obtained after Mesylate Type A was suspended for 1 and 24 hours in H₂O (slightly turbid was observed after suspending for 4 hours, but the sample was not enough for XRPD test). After Mesylate Type A was suspended for 1, 4 and 24 hours in SGF, clear solutions were obtained. Clear solutions were not obtained after Mesylate Type A was suspended for 1, 3 or 24 hours in FaSSIF.

TABLE 1.5
Summary of Kinetic Solubility Results at 37° C.

| Crystal | | | | | | | | | |
|---|---|---|---|---|---|---|---|---|---|
| Form | | | | | | | | | |
| (Batch | 1 hr | | | 4 hrs | | | 24 hrs | | |
| No.) | S | pH | FC | S | pH | FC | S | pH | FC |

Kinetic Solubility in H₂O at 37° C.

| | | | | | | | | | |
|---|---|---|---|---|---|---|---|---|---|
| Freebase | 0.0012 | 8.8 | Yes | 0.0012 | 8.6 | Yes | 0.0024 | 8.4 | Yes |
| Type | | | | | | | | | |
| E + F | | | | | | | | | |
| (818718- | | | | | | | | | |
| 01-A) | | | | | | | | | |
| L-Malate | 7.1 | 4.0 | NA | 7.2 | 4.0 | NA | 6.7 | 4.1 | NA |
| Type B | | | | | | | | | |
| (818718- | | | | | | | | | |
| 21-A1) | | | | | | | | | |
| Mesylate | 7.5 | 6.1 | NA | 7.5 | 6.2 | — | 7.4 | 6.0 | NA |
| Type A | | | | | | | | | |
| (818718-2 | | | | | | | | | |
| 1-A5) | | | | | | | | | |

Kinetic Solubility in SGF at 37° C.

| | | | | | | | | | |
|---|---|---|---|---|---|---|---|---|---|
| Freebase | 7.1 | 5.4 | Yes | 7.2 | 5.4 | Yes | 7.2 | 6.2 | Yes |
| Type | | | | | | | | | |
| E + F | | | | | | | | | |
| (818718- | | | | | | | | | |
| 01-A) | | | | | | | | | |
| L-Malate | 7.6 | 3.2 | NA | 7.7 | 3.1 | NA | 6.9 | 3.2 | NA |
| Type B | | | | | | | | | |
| (818718- | | | | | | | | | |
| 21-A1) | | | | | | | | | |
| Mesylate | 8.2 | 2.5 | NA | 8.0 | 2.6 | NA | 7.4 | 2.5 | NA |
| Type A | | | | | | | | | |
| (818718- | | | | | | | | | |
| 21-A5) | | | | | | | | | |

Kinetic Solubility in FaSSIF at 37° C.

| | | | | | | | | | |
|---|---|---|---|---|---|---|---|---|---|
| Freebase | 0.17 | 6.6 | Yes | 0.18 | 5.6 | Yes | 0.11 | 6.5 | Yes |
| Type | | | | | | | | | |
| E + F | | | | | | | | | |
| (818718- | | | | | | | | | |
| 01-A) | | | | | | | | | |
| L-Malate | 7.2 | 5.0 | NA | 6.9 | 4.9 | NA | 7.4 | 4.9 | NA |
| Type B | | | | | | | | | |
| (818718- | | | | | | | | | |
| 21-A1) | | | | | | | | | |
| Mesylate | 0.37 | 6.4 | Yes | 0.36 | 6.3 | Yes | 0.34 | 6.2 | Yes |
| Type A | | | | | | | | | |
| (818718- | | | | | | | | | |
| 21-A5) | | | | | | | | | |

S: solubility in mg/mL; pH: final pH, FC: form change; —: due to limited solids, the data ofXRPD were not collected; NA: clear solutions were obtained.

Solid-state Stability. Solid-state stability of Freebase Type E+F (818718-01-A), L-Malate Type B (818718-21-A1) and Mesylate Type A (818718-21-A5) were evaluated under 25° C./60% RH and 40° C./75% RH (open) for two weeks. Stability samples were characterized by XRPD to check any solid form change, and by HPLC to check purity change. The results are summarized in Table 1.6. XRPD data showed that Freebase Type E+F, L-Malate Type B and Mesylate Type A showed good physicochemical stability under the tested conditions as evidenced by no form change or substantial HPLC purity decrease after storage.

TABLE 1.6
Summary of Two-week Solid-state Stability

| | Initial | | | | |
|---|---|---|---|---|---|
| Solid form | Purity | | Purity | Purity vs | |
| (Batch No.) | (area %) | Condition | (area %) | Initial (%) | Final Form |
| Freebase Type | 98.63 | 25° C./60% RH | 98.64 | 100.0 | Freebase Type |
| E + F | | 40° C./75% RH | 98.75 | 100.1 | E + F |
| (818718-01-A) | | | | | |
| L-Malate Type B | 98.93 | 25° C./60% RH | 98.97 | 100.0 | L-Malate Type |
| (818718-21-A1) | | 40° C./75% RH | 98.96 | 100.0 | B |
| Mesylate Type A | 99.26 | 25° C./60% RH | 99.31 | 100.1 | Mesylate Type |
| (818718-21-A5) | | 40° C./75% RH | 99.43 | 100.2 | A |

Mesylate and freebase were submitted to polymorph screening. Based on investigation results, no new mesylate form except for hydrous Mesylate Type A was discovered during polymorph screening. For freebase, six crystalline hits were obtained, including anhydrates Freebase Type B/E/F/G, hydrate Freebase Type C and DCM solvate Freebase Type D. It is noteworthy that the freebase starting material (818718-01-A) used for salt screening, which was initially assigned as Freebase Type A, was determined to be a mixture of Freebase Type E+F.

Mesylate Type A (818718-48-A) was re-prepared using a new batch of freebase material (818718-45-A) using the detailed preparation procedure shown in Table 1.7, by slurry of a mixture of methanesulfonic acid and freebase (molar ratio 1:1) in EtOAc at RT. No form change was observed after vacuum drying. The data of TGA and DSC showed a weight loss of 2.0% up to 150° C., and two thermal signals at 61.4° C. and 265.1° C. (peak temperature; peak onset occurred at 262.4° C.). Around 0.07 molar EtOAc (about 0.95 weight percent) was detected by ¹H NMR. As shown by PLM, Mesylate Type A (818718-48-A) was composed of rod-like and needle-like particles. Based on the integrals, the stoichiometric ratio of methanesulfonic acid and freebase was determined to be 1.01. The HPLC purity of Mesylate Type A (818718-48-A) was determined to be 99.84 area percent. This batch of Mesylate Type A was used for polymorph screening experiments. As a result, only one crystalline hit of Mesylate Type A was observed from polymorph screening experiments.

TABLE 1.7
Preparation Procedure of Mesylate Type A

Crystal

| Form | Preparation Procedures | |
|---|---|---|
| Mesylate | 1. | Add 5.0975 g Freebase Type B (818718-45-A) and |
| Type A | | 100.0 mL EtOAc to a 250-mL reactor. A suspension |
| (818718- | | was obtained. |
| 48-A) | 2. | Add 608.64 μL methanesulfonic acid to 30 mL EtOAc |
| | | in a 100-mL glass vial, and add the solution of acid |
| | | into the suspension of freebase. |
| | 3. | Sample for XRPD after stirring for about 26 hours at |
| | | RT, and the pattern conformed to Mesylate Type A. |
| | 4. | Filter the suspension obtained and vacuum dry the wet |
| | | cake at RT for about 6.5 hours. |
| | 5. | Collect solids of 5.4823 g (yield: 91.52%). |

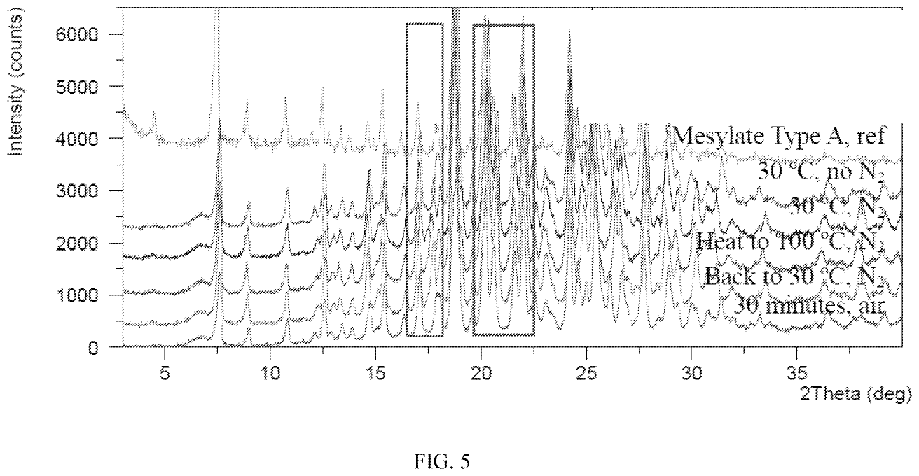

To identify Mesylate Type A, Mesylate Type A was tested by VT-XRPD. The XRPD results shown in FIG. 5 showed diffraction peak shift (shown in the box) was observed after the sample was blown under N2 for twenty minutes, which might be caused by dehydration. No significant further change was observed after heating the sample to 100° C. or cooled back to 30° C. under N2. After the sample was re-exposed to ambient conditions for thirty minutes, Mesylate Type A was obtained again. Thus, Mesylate Type A was speculated to be capable of forming a hydrate, and absorbing water after dehydration.

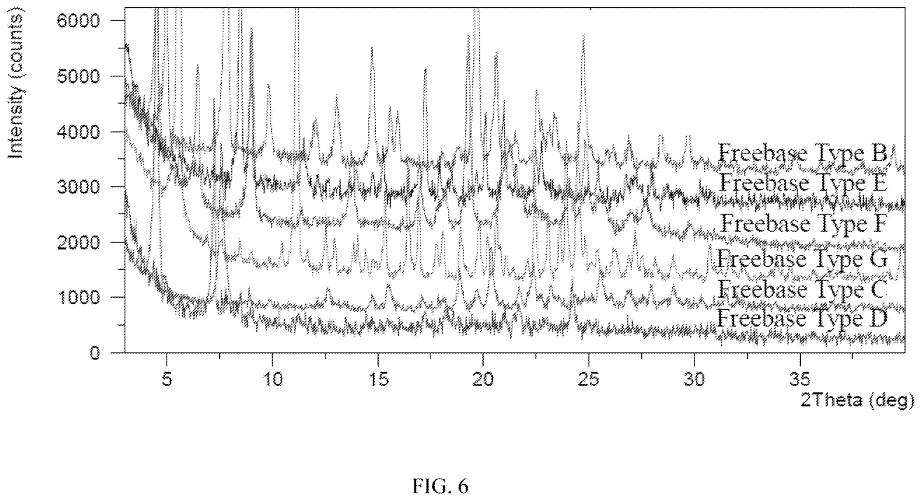

A new batch of freebase (SY18002581-12) was used for polymorph screening. XRPD result showed that the new batch (818718-45-A) was crystalline with a different XRPD pattern from previous batch (SY18002581-8), so this new form was assigned as Freebase Type B. Using Freebase Type B as starting material, a total of 67 polymorph screening experiments were conducted. As a result, anhydrates Freebase Type B/E/F/G, hydrate Freebase Type C and DCM solvate Freebase Type D were discovered with characterization data and XRPD patterns shown in Table 1.8 and FIG. 6, respectively.

TABLE 1.8
Characterization of Freebase Forms

| | | | | Endo- | | |
|---|---|---|---|---|---|---|
| | | | Weight | therm | | Solvent |
| | | Sample | Loss | (peak, | Purity | Residual |
| Crystal Form | | ID | (%) | ° C.) | (area %) | (wt %) |
| Anhydrate | Freebase | 818718- | 0.7 | 152.8 | 99.49 | / |
| | Type B | 45-A | (125° C.) | | | |
| Anhydrate | Freebase | 818718- | 2.0 | 80.9, | 99.70 | 0.3 |
| | Type E | 60-A3 | (125° C.) | 163.3 | | (acetone) |
| Anhydrate | Freebase | 818718- | 2.6 | 49.5, | 99.73 | 1.2* |
| | Type F | 60-A7 | (125° C.) | 154.5, | | (THF) |
| | | | | 161.3 | | |
| Anhydrate | Freebase | 818718- | 2.5 | 174.4 | 99.96 | NA |
| | Type G | 81-B2 | (125° C.) | | | |
| Hydrate | Freebase | 818718- | 3.3 | 81.7, | 99.82 | NA |
| | Type C | 61-A2 | (125° C.) | 151.6 | | |
| Solvate | Freebase | 818718- | 7.8 | 59.3, | 98.70 | 4.3 |
| (DCM) | Type D | 63-A2 | (125° C.) | 95.4, | | (DCM) |
| | | | | 143.0, | | |
| | | | | 159.7 | | |

*the data was collected of another batch of Freebase Type F (818718-76-A3). After the sample was heated to 125° C., no solvent residual (THF) was detected in ¹H NMR. Details could refer to section 6.7.3.

Thermodynamic stability relationship among Freebase Type B, C, E, F and G were investigated by slurry competition experiments. As a result: a) in IPAc systems from 5° C. to 50° C. and in acetone/H₂O with different a_{w} systems at RT, Freebase Type E was observed; b) there might be a potential metastable form, such as EtOH or ACN solvate, in ACN, EtOH/H₂O (a_{w}=0.2) and EtOH systems, which might turn to Freebase Type C or G quickly.

To further examine the stability of Mesylate Type A, XRPD spectra were obtained from a sample of Mesylate Type A stored for six months at 25° C. and 600% relative humidity in a closed container, and a sample of Mesylate Type A stored for six months at 40° C. and 7500 relative humidity in a closed container. FIG. 1 shows the XPRD spectrum of Mesylate Type A stored for six months at 25° C. and 600% relative humidity in a closed container, and Table 1.9 lists various data associated with the XRPD spectrum of FIG. 1. FIG. 7 shows the XPRD spectrum of Mesylate Type A stored for six months at 40° C. and 7500 relative humidity in a closed container, and Table 1.10 lists various data associated with the XRPD spectrum of FIG. 7.

TABLE 1.9
XRPD Characterization of Mesylate
Type A Stored At 25° C. and 60% RH

| Name | Caption (display) | d Value | Rel. Intensity |
|---|---|---|---|
| Peak #1 | 4.405° | 20.04412 Å | 14.0% |
| Peak #2 | 7.464° | 11.83435 Å | 100.0% |
| Peak #3 | 8.798° | 10.04270 Å | 20.1% |
| Peak #4 | 10.658° | 8.29412 Å | 12.1% |
| Peak #5 | 12.437° | 7.11115 Å | 13.8% |
| Peak #6 | 14.521° | 6.09496 Å | 12.4% |
| Peak #7 | 15.246° | 5.80680 Å | 12.0% |
| Peak #8 | 16.907° | 5.23989 Å | 14.1% |
| Peak #9 | 18.650° | 4.75400 Å | 35.6% |
| Peak #10 | 20.193° | 4.39406 Å | 17.5% |
| Peak #11 | 20.570° | 4.31426 Å | 12.2% |
| Peak #12 | 21.892° | 4.05668 Å | 16.9% |
| Peak #13 | 24.081° | 3.69272 Å | 17.3% |
| Peak #14 | 25.236° | 3.52620 Å | 19.6% |

TABLE 1.10
XRPD Characterization of Mesylate
Type A Stored At 40° C. and 75% RH

| Name | Caption (display) | d Value | Rel. Intensity |
|---|---|---|---|
| Peak #1 | 4.363° | 20.23456 Å | 13.6% |
| Peak #2 | 7.392° | 11.94951 Å | 100.0% |
| Peak #3 | 8.763° | 10.08316 Å | 22.6% |
| Peak #4 | 10.608° | 8.33277 Å | 12.2% |
| Peak #5 | 12.379° | 7.14465 Å | 24.8% |
| Peak #6 | 14.480° | 6.11224 Å | 13.2% |
| Peak #7 | 15.202° | 5.82365 Å | 17.3% |
| Peak #8 | 16.873° | 5.25043 Å | 18.3% |
| Peak #9 | 18.598° | 4.76715 Å | 38.5% |
| Peak #10 | 19.884° | 4.46149 Å | 11.0% |
| Peak #11 | 20.066° | 4.42144 Å | 17.4% |
| Peak #12 | 20.432° | 4.34308 Å | 13.5% |
| Peak #13 | 21.416° | 4.14576 Å | 14.8% |
| Peak #14 | 21.825° | 4.06901 Å | 31.5% |
| Peak #15 | 24.030° | 3.70043 Å | 24.1% |
| Peak #16 | 24.804° | 3.58662 Å | 12.1% |
| Peak #17 | 25.193° | 3.53213 Å | 22.8% |
| Peak #18 | 26.453° | 3.36668 Å | 12.4% |
| Peak #19 | 27.586° | 3.23087 Å | 11.5% |
| Peak #20 | 28.661° | 3.11215 Å | 11.5% |

In conclusion, salt screening was conducted on freebase form of the compound of structural formula I. A total of 35 crystalline salt hits were obtained from 84 salt screening experiments. Mesylate Type A and L-Malate Type B were selected as salt leads for further evaluation, including hygroscopicity, kinetic solubility in different bio-relevant media, and solid-state stability using freebase as control. Based on the characterization and evaluation results, both mesylate and freebase were chosen for polymorph screening. For mesylate, no new mesylate form except for hydrate Mesylate Type A was discovered from polymorph screening. For freebase, anhydrates Freebase Type B/E/F/G, hydrate Freebase Type C and DCM solvate Freebase Type D were discovered. Slurry competition experiments results among Freebase Type B, C, D, E, F and G showed Freebase Type E was observed in IPAc systems and acetone/H2O with different a_{w} systems. There might be a potential metastable EtOH or ACN solvate in ACN, EtOH/H2O (a_{w}=0.2) and EtOH systems, which might turn to Freebase Type C or G quickly.

Based on the collected data from screening and evaluation, Mesylate Type A showed good physiochemical stability and low polymorphism risk.

Instruments and Methods:

XRPD Analysis. For XRPD analysis, PANalytical X-ray powder diffractometers in reflection mode were used. The XRPD parameters are listed in Table 1.11.

TABLE 1.11
Parameters for XRPD

| | |
|---|---|
| Parameters | Reflection Mode |

| Model | Empyrean | X′ Pert³ |
|---|---|---|
| X-Ray wavelength | Cu, kα, | |
| | Kα1 (Å): 1.540598 | |
| | Kα2 (Å): 1.544426 | |
| | Kα2/Kα1 intensity ratio: 0.50 | |
| X-Ray tube setting | 45 kV, 40 mA | |
| Scan mode | Continuous | |
| Scan range (°2 Theta) | 3°-40° | |
| Divergence slit | Automatic | ⅛° |
| Scan step time (s) | 17.8 | 46.7 |
| Step size (°2 Theta) | 0.0167 | 0.0263 |
| Test Time | 5 min 30 s | 5 min 4 s |

TGA and DSC. TGA data were collected using a TA Q5000 TGA or TA Discovery TGA5500 from TA Instruments and DSC was performed using a TA Discovery DSC2500 from TA Instruments. Detailed parameters used are listed in Table 1.12.

TABLE 1.12
Parameters for TGA and DSC

| Parameters | TGA | DSC |
|---|---|---|
| Method | Ramp | Ramp |
| Sample pan | Aluminum, open | Aluminum, crimped |
| Temperature | RT - desired temperature | 25° C. - desired temperature |
| Heating rate | 10° C./min | 10° C./min |
| Purge gas | N₂ | N₂ |

HPLC. Agilent 1290 UPLC was utilized, and detailed chromatographic conditions for purity test are listed in Table 1.13.

TABLE 1.13
Chromatographic Conditions and Parameters for HPLC

| Parameters | Value | |
|---|---|---|
| Mobile phase | A: 0.05% TFA in H₂O | |
| | B: 0.05% TFA in acetonitrile | |
| | Time (min) | % B |
| | 0.0 | 5 |
| | 6.0 | 40 |
| Gradient table | 8.0 | 95 |
| | 8.1 | 5 |
| | 10.0 | 5 |

| | | |
|---|---|---|
| Run time | 10.0 | min |
| Post time | 0.0 | min |
| Flow rate | 0.5 | mL/min |
| Injection volume | 5 | μL |

| | |
|---|---|
| Detector wavelength | UV at 220 nm |
| Column temperature | 40° C. |
| Sampler temperature | RT |
| Diluent | 50% ACN or ACN:H₂O (1:5, v:v) |

IC. Ion chromatography (IC) method for counter anion ion content measurement to determine stoichiometric ratio is listed in Table 1.14.

TABLE 1.14
IC Method for Counter Anion Ion Content Measurement

| Parameters | Value |
|---|---|
| Column | IonPac AS18 Analytical Column (4 × 250 mm) |
| Mobile Phase | 25 mM NaOH |
| Injection volume | 25 μL |
| Flow rate | 1.0 mL/min |
| Cell temperature | 35° C. |
| Column temperature | 35° C. |
| Current | 80 mA |
| Run Time | 8 (C1⁻) mins or 11 (SO₄²⁻) mins or 13 (Br⁻) mins or |
| | 16 (C₂O₄²⁻) mins or 40 (PO₄³⁻) mins |

DVS. Dynamic Vapor Sorption (DVS) was measured via a SMS (Surface Measurement Systems) DVS Intrinsic. The relative humidity at 25° C. was calibrated against deliquescence point of LiCl, Mg(NO3)2 and KCl. Actual parameters for DVS test are listed in Table 1.15.

TABLE 1.15
Parameters for DVS

| Parameters | Value |
|---|---|
| Temperature | 25° C. |
| Sample size | 10~20 mg |
| Gas and flow rate | N₂, 200 mL/min |
| dm/dt | 0.002%/min |
| Min. dm/dt stability duration | 10 min |
| Max. equilibrium time | 180 min |
| RH range | 0% RH to 95% RH |
| RH step size | 10% RH from 0% RH to 90% RH |
| | 5% RH from 90% RH to 95% RH |

Solution NMR. Solution NMR was collected on Bruker 400M NMR Spectrometer using DMSO-d₆ as solvent.

### Example 2. Capsule Formulation Design of Experiments (DOE)

The formulation development work started with a six-factor, four-response DOE. As shown in Table 2.1, eight formulations were prepared to evaluate the following six factors:
Filler type (mannitol or lactose);
Disintegrant level;
Lubricant type (sodium stearyl fumarate or magnesium stearate);
Glidant level;
Microcrystalline cellulose (MCC)-to-filler ratio; and
Capsule size (1 mg or 50 mg).

TABLE 2.1
DOE Round 1 Formulations

| | Density | | | | | | | | |
|---|---|---|---|---|---|---|---|---|---|
| | (g/cc) | Run 1 | Run 2 | Run 3 | Run 4 | Run 5 | Run 6 | Run 7 | Run 8 |
| Compound of | 0.21 | 22.40 | 1.02 | 1.02 | 22.40 | 1.02 | 22.40 | 22.40 | 1.02 |
| Structural | | | | | | | | | |
| Formula (I) | | | | | | | | | |
| (%) | | | | | | | | | |
| Microcrystalline | 0.33 | 33.30 | — | 43.24 | — | 46.74 | — | 23.12 | — |
| Cellulose | | | | | | | | | |
| (Avicel PH- | | | | | | | | | |
| 200) (%) | | | | | | | | | |
| Mannitol | 0.45 | 33.30 | 94.98 | — | 65.10 | 46.74 | — | — | — |
| (Pearlitol SD | | | | | | | | | |
| 100) (%) | | | | | | | | | |
| Lactose (Fast | 0.55 | — | — | 43.24 | — | — | 72.10 | 46.23 | 87.98 |
| Flo 316) (%) | | | | | | | | | |
| Ac-Di-Sol (%) | 0.53 | 10.00 | 3.00 | 10.00 | 10.00 | 3.00 | 3.00 | 6.50 | 10.00 |
| Cab-O-Sil (%) | 0.05 | 0.50 | 0.50 | 2.00 | 2.00 | 2.00 | 2.00 | 1.25 | 0.50 |
| Magnesium | 0.17 | — | 0.50 | 0.50 | 0.50 | — | — | 0.50 | — |
| Stearate (%) | | | | | | | | | |
| Sodium | 0.28 | 0.50 | — | — | — | 0.50 | 0.50 | — | 0.50 |
| Stearyl | | | | | | | | | |
| Fumarate (%) | | | | | | | | | |
| Total (%) | | 100.00 | 100.00 | 100.00 | 100.00 | 100.00 | 100.00 | 100.00 | 100.00 |
| API Potency | | 98.21 | 98.21 | 98.21 | 98.21 | 98.21 | 98.21 | 98.21 | 98.21 |
| (%) | | | | | | | | | |
| Blend Potency | | 220 | 10 | 10 | 220 | 10 | 220 | 220 | 10 |
| Capsule Dose | | 50 | 1 | 1 | 50 | 1 | 50 | 50 | 1 |
| (mgA) | | | | | | | | | |
| Capsule Fill | | 227.3 | 100.0 | 100.0 | 227.3 | 100.0 | 227.3 | 227.3 | 100.0 |
| Weight (mg) | | | | | | | | | |
| Capsule Size | | 1 | 4 | 4 | 1 | 4 | 1 | 1 | 4 |
| Capsule | | 0.50 | 0.21 | 0.21 | 0.50 | 0.21 | 0.50 | 0.50 | 0.21 |
| Volume (mL) | | | | | | | | | |
| Filled Capsule | | 0.45 | 0.48 | 0.48 | 0.45 | 0.48 | 0.45 | 0.45 | 0.48 |
| Density | | | | | | | | | |
| (g/mL) | | | | | | | | | |

*Note:
Potency correction used for API. Used potency of 98.21% (subtracting residual solvent, water and impurities). Dose includes mesylate salt.

The formulations were evaluated using the following four responses (see Table 2.2):
Disintegration time;
Bulk density;
Flowability; and
Weight variation.

TABLE 2.2
DOE Round 1 Results

| | | | | | | | Disintegration | | | |
|---|---|---|---|---|---|---|---|---|---|---|
| | | | | | | | Time | Bulk | | Weight |
| | Filler | Disintegrant | Lubricant | Glidant | Capsule | MCC:Filler | (average, | Density | | Variation |
| Run | Type | level | type | level | Size | Ratio | min) | (g/cc) | FF | (% RSD) |
| 1 | Mannitol | 10 | SSF | 0.5 | 50 | 1 | 2.0 | 0.426 | 3.48 | 1.48 |
| 2 | Mannitol | 3 | MgSt | 0.5 | 1 | 0 | 2.0 | 0.517 | 4.90 | 2.79 |
| 3 | Lactose | 10 | MgSt | 2 | 1 | 1 | 1.6 | 0.500 | 4.17 | 3.83 |
| 4 | Mannitol | 10 | MgSt | 2 | 50 | 0 | 2.1 | 0.391 | 3.22 | 1.77 |
| 5 | Mannitol | 3 | SSF | 2 | 1 | 1 | 1.4 | 0.414 | 4.56 | 2.22 |
| 6 | Lactose | 3 | SSF | 2 | 50 | 0 | 2.4 | 0.441 | 3.28 | 3.21 |
| 7 | Lactose | 6.5 | MgSt | 1.25 | 50 | 0.5 | 3.9 | 0.441 | 3.93 | 2.72 |
| 8 | Lactose | 10 | SSF | 0.5 | 1 | 0 | 1.9 | 0.618 | 4.83 | 2.90 |

The results of the DOE were analyzed with an ANOVA to determine which factors had statistically significant (p-value<0.1) impact on the different responses. The DOE analysis is shown in Table 2.3.

TABLE 2.3
DOE Round 1 Data Analysis

| | Disintegration | | | Weight |
|---|---|---|---|---|
| | Time | Bulk Density | | Variation |
| | (average, min) | (g/cc) | FF | (% RSD) |
| Filler Type | | P-value = 0.03 | | P-value = 0.01 |
| (Mannitol or | | | | |
| Lactose) | | | | |
| Disintegrant | | | | |
| Level | | | | |
| Lubricant Type | | | | |
| (SSF or MgSt) | | | | |
| Glidant Level | | P-value = 0.03 | P-value = 0.08 | |
| Capsule Size | P-value = 0.10 | P-value = 0.02 | P-value = 0.001 | P-value = 0.08 |
| (50 mg or 1 mg) | | | | |
| MCC:Filler | | P-value = 0.08 | | |
| Ratio | | | | |

Based upon these results, mannitol was selected as the filler, a disintegrant level of 5% was selected, sodium stearyl fumarate was selected as the lubricant, a glidant level of 0.5% was selected, and an MCC-to-Filler ratio of 1:1 was selected.

Four additional formulations were manufactured using the above selections to refine the formulation in a second round of DOE experiments. In the second round, the focus was on the blend characterization responses (flowability and bulk density) and the capsule characterization responses (weight uniformity and content uniformity). The content uniformity test was added at this stage of the development to ensure manufacturing feasibility, particularly for the 1 mg capsules. DOE round 2 formulations are shown in Table 2.4.

TABLE 2.4
DOE Round 2 Formulations

| | Density | | | | |
|---|---|---|---|---|---|
| | (g/cc) | Run 9 | Run 10 | Run 11 | Run 12 |
| Compound of Structural Formula | 0.21 | 26.47 | 1.12 | 1.12 | 1.12 |
| (I) (%) | | | | | |
| Microcrystalline Cellulose | 0.33 | 33.76 | 46.44 | | 46.44 |
| (Avicel PH-200) (%) | | | | | |
| Microcrystalline Cellulose | 0.25 | | | 46.44 | |
| (Avicel PH-105) (%) | | | | | |
| Mannitol (Pearlitol SD 100) (%) | 0.45 | 33.76 | 46.44 | | 46.44 |
| Mannitol (Pearlitol 50 C) (%) | 0.55 | | | 46.44 | |
| Lactose (Fast Flo 316) (%) | 0.55 | — | — | — | — |
| Ac-Di-Sol (%) | 0.53 | 5.00 | 5.00 | 5.00 | 5.00 |
| Cab-O-Sil (%) | 0.05 | 0.50 | 0.50 | 0.50 | 0.50 |
| Magnesium Stearate (%) | 0.17 | — | — | — | — |
| Sodium Stearyl Fumarate (%) | 0.28 | 0.50 | 0.50 | 0.50 | 0.50 |
| Total (%) | | 100.00 | 100.00 | 100.00 | 100.00 |
| API Potency (%) | | 98.21 | 98.21 | 98.21 | 98.21 |
| Blend Potency | | 260 | 11 | 11 | 11 |

*Note: Potency correction used for API. Used potency of 98.21% (subtracting residual
solvent, water and impurities). Dose includes mesylate salt.

| | | | | |
|---|---|---|---|---|
| Capsule Dose (mgA) | 50 | 1 | 1 | 1 |
| Capsule Fill Weight (mg) | 192.3 | 90.9 | 90.9 | 90.9 |
| Capsule Size | 1 | 4 | 4 | 4 |
| Capsule Volume (mL) | 0.50 | 0.21 | 0.21 | 0.21 |
| Filled Capsule Density (g/mL) | 0.38 | 0.43 | 0.43 | 0.43 |
| Formulation Density (g/mL) | 0.35 | 0.39 | 0.40 | 0.39 |

The round 2 formulations were evaluated using the same responses as those from Round 1, and the results of both Rounds 1 and 2 are shown in Table 2.5. The content uniformity data are shown separately in Table 2.6, and indicated that it would not be feasible to manufacture the 1 mg formulations. Based upon the data in Tables 2.5 and 2.6, it was determined that higher active pharmaceutical ingredient (API) loading would be needed in the final formulation, but that the prior parameter selections (e.g., mannitol) were appropriate.

TABLE 2.5
DOE Round 1 and 2 Results

| | | | | | | | Disintegration | Bulk | | Weight |
|---|---|---|---|---|---|---|---|---|---|---|
| | | Disintegrant | Lubricant | Glidant | Capsule | MCC:Filler | Time (average, | Density | | Variation |
| Run | Filler Type | level | type | level | Size | Ratio | min) | (g/cc) | FF | (% RSD) |
| 1 | Mannitol | 10 | SSF | 0.5 | 50 | 1 | 2.0 | 0.426 | 3.48 | 1.48 |
| 2 | Mannitol | 3 | MgSt | 0.5 | 1 | 0 | 2.0 | 0.517 | 4.90 | 2.79 |
| 3 | Lactose | 10 | MgSt | 2 | 1 | 1 | 1.6 | 0.500 | 4.17 | 3.83 |
| 4 | Mannitol | 10 | MgSt | 2 | 50 | 0 | 2.1 | 0.391 | 3.22 | 1.77 |
| 5 | Mannitol | 3 | SSF | 2 | 1 | 1 | 1.4 | 0.414 | 4.56 | 2.22 |
| 6 | Lactose | 3 | SSF | 2 | 50 | 0 | 2.4 | 0.441 | 3.28 | 3.21 |
| 7 | Lactose | 6.5 | MgSt | 1.25 | 50 | 0.5 | 3.9 | 0.441 | 3.93 | 2.72 |
| 8 | Lactose | 10 | SSF | 0.5 | 1 | 0 | 1.9 | 0.618 | 4.83 | 2.90 |
| 9 | Mannitol | 5 | SSF | 0.5 | 50 | 1:1 | 2.5 | 0.374 | 3.06 | 1.89 |
| 10 | Mannitol | 5 | SSF | 0.5 | 1 | 1:1 | 2.0 | 0.505 | 4.83 | 3.33 |
| 11 | Mannitol | 5 | SSF | 0.5 | 1 | 1:1 | 2.2 | 0.428 | 2.91 | 4.37 |
| 12 | Mannitol | 5 | SSF | 0.5 | 1 | 1:1 | 2.1 | 0.453 | 5.23 | 2.64 |

TABLE 2.6
DOE Round 2 Content Uniformity Data

| | Avg. | | | USP <905> Acceptance |
|---|---|---|---|---|
| Sample | Potency | Standard | | Value |
| Description | (% LC) | Deviation | RSD | (<15 is passing) |
| 50 mg Run 9 | 102.1 | 2.7 | 0.0 | 7.1 |
| 1 mg Run 10 | 107.7 | 14.5 | 0.1 | 40.9 |
| 1 mg Run 12 | 100.9 | 14.5 | 0.1 | 34.7 |
| 1 mg Run 11 | 105.3 | 12.5 | 0.1 | 33.9 |

### Example 3. Roller Compaction Development

To determine the feasibility of using roller compaction to fill capsules with greater drug load (of 50 mg, 100 mg, and 200 mg), blends were prepared based on the blends used in Example 2 and calculations shown in Table 3.1 to obtain 350 drug load in the granulation. A granulation bulk density of about 0.5 g/mL was targeted for development. The granulation was formulated into capsules. See Table 3.2.

TABLE 3.1

| | Blend for 7 and 28 mg | | Roller Compaction Development | | |
|---|---|---|---|---|---|
| | 7 mg | 28 mg | 50 mg | 100 mg | 200 mg |
| Structural Formula I | 8.15% | 19.35% | 35.00% | 35.00% | 35.00% |
| Silicified MCC (SMCC 90) | 43.18% | 37.58% | 29.75% | 29.75% | 29.75% |
| Mannitol (Pearlitol SD 100) | 43.18% | 37.58% | 29.75% | 29.75% | 29.75% |
| Ac-Di-Sol | 5.00% | 5.00% | 5.00% | 5.00% | 5.00% |
| Sodium Stearyl Fumarate | 0.50% | 0.50% | 0.50% | 0.50% | 0.50% |
| Total | 100.00% | 100.00% | 100.00% | 100.00% | 100.00% |

TABLE 3.2

| | | | |
|---|---|---|---|
| Capsule Dose (mg) | 50 | 100 | 200 |
| Capsule Fill Weight (mg) | 146 | 291 | 582 |
| Capsule Size | 4 | 1 | 00 |
| Capsule Volume (mL) | 0.21 | 0.50 | 0.91 |
| Filled Capsule Density (g/mL) | 0.69 | 0.58 | 0.64 |

### Example 4. Four-Week Stability Studies

Materials: 50 mg capsule (Lot #: T12-704-52); 100 mg capsule (Lot #: T12-704-54); 200 mg capsule (Lot #: T12-704-53).

Methods: Samples were stored (closed) at 40° C. and 7500 relative humidity (RH). Appearance, water content, assay and related substances (HPLC) were tested (n=6), sink dissolution.

Results: Appearance, water content, and assay and related substances remained similar to t=0. As seen in Table 4.1, at t=4 weeks, capsules contained 2.6-2.8% by weight water, which is comparable to t=0. Also, appearance remained the same as at t=0 of the study. As seen in Table 4.2, assay values were comparable to t=0; impurity profile matched that of the API standard. The 4-week diluent peak was excluded from the impurity workup.

TABLE 4.1
Appearance and Water Content

| | | | Water Content | |
|---|---|---|---|---|
| | | Appearance | (wt %) | |
| Sample | | t = 0 and 4 wks 40/75 | | 4 wks 40/75 |
| Description | Lot | Closed | t = 0 | Closed |
| 50 mg | T12- | Off white capsule shell, | 2.72 | 2.75 |
| Capsules | 704-52 | contents are an off-white | | |
| | | powder | | |
| 200 mg | T12- | Off white capsule shell, | 2.58 | 2.58 |
| Capsules | 704-53 | contents are an off-white | | |
| | | powder | | |
| 100 mg | T12- | Off white capsule shell, | 2.58 | 2.64 |
| Capsules | 704-54 | contents are an off-white | | |
| | | powder | | |

TABLE 4.2
4 Week Stability Characterization

| | | | | | | | 100 mg |
|---|---|---|---|---|---|---|---|
| | | Capsules, | 50 mg | Capsules, | 200 mg | Capsules, | Capsule, |
| | Structural | 50 mg | Capsule, 4 | 200 mg | Capsule, 4 | 100 mg | 4 wks |
| | Formula I | Lot: T12- | wks 40/75 | Lot: T12- | wks 40/75 | Lot: T12- | 40/75 |
| RRT | Lot: 1912515057 | 704-52 | Closed | 704-53 | Closed | 704-54 | Closed |
| 0.84 | Detected, | Detected, | | Detected, | | | |
| | <0.05% | <0.05% | | <0.05% | | | |
| 0.89 | 0.15% | 0.10% | 0.14% | 0.08% | 0.11% | 0.09% | 0.09% |
| 0.97 | 0.15% | 0.14% | 0.15% | 0.15% | 0.20% | 0.14% | 0.14% |
| 1.08 | 0.11% | 0.10% | 0.10% | 0.11% | 0.11% | 0.11% | 0.11% |
| 1.13 | Detected, | Detected, | | | | | |
| | <0.05% | <0.05% | | | | | |
| 1.20 | 0.09% | 0.10% | 0.11% | 0.09% | 0.11% | 0.09% | 0.10% |
| 1.21 | 0.05% | 0.06% | 0.06% | 0.05% | 0.06% | 0.05% | 0.05% |
| 1.24 | 0.08% | 0.08% | 0.09% | 0.08% | 0.08% | 0.08% | 0.08% |
| 1.29 | Detected, | | | | | | |
| | <0.05% | | | | | | |
| 1.33 | Detected, | Detected, | Detected, | Detected, | Detected, | Detected, | |
| | <0.05% | <0.05% | <0.05% | <0.05% | <0.05% | <0.05% | |
| Total | 0.63% | 0.57% | 0.66% | 0.57% | 0.68% | 0.56% | 0.58% |
| Impurities | | | | | | | |
| Assay | NA | 97.8 | 96.4 | 96.7 | 95.5 | 95.4 | 96.6 |
| (%LC) | | | | | | | |

### Example 5. Representative Synthesis of the Compound of Structural Formula I

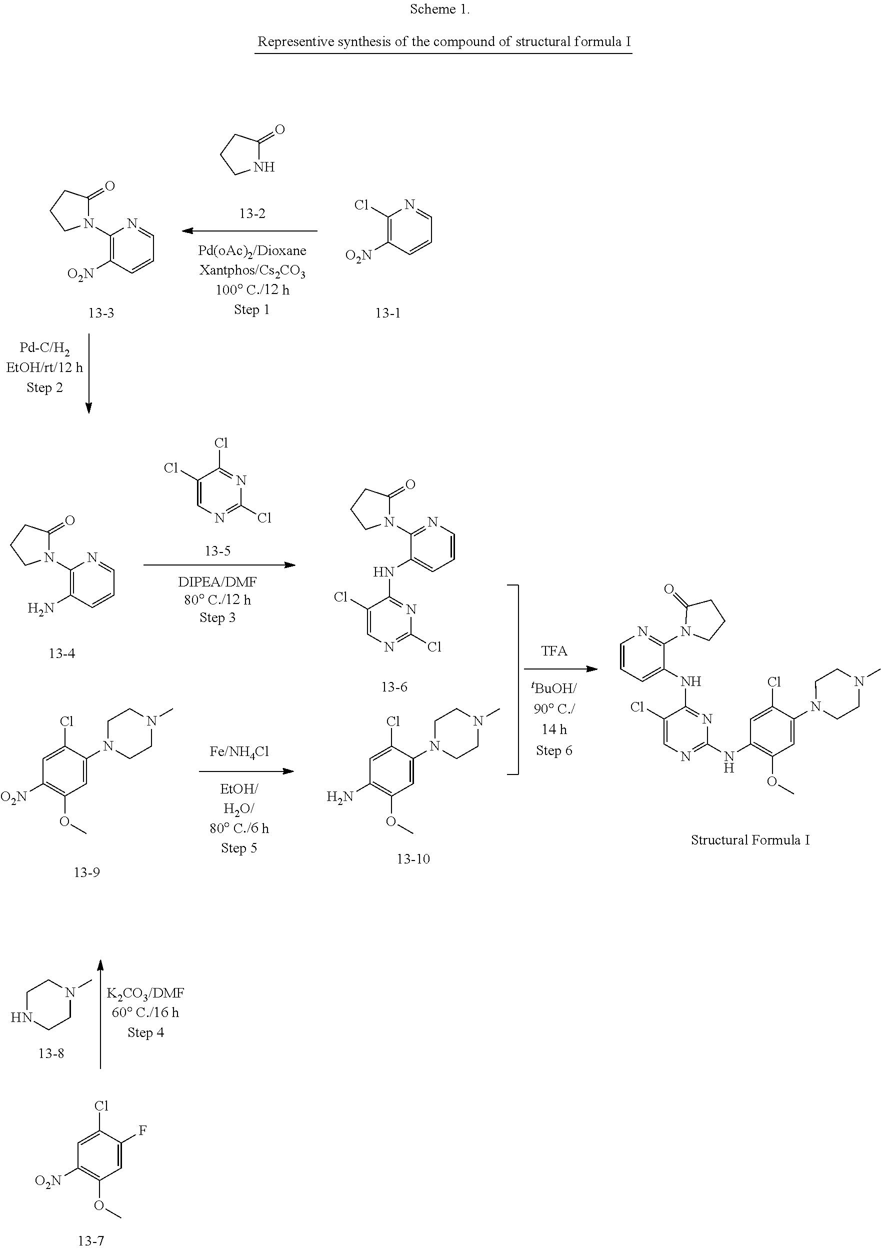

In step 1, to a dioxane solution (80 ml) of 2-chloro-3-nitro pyridine (10.0 g, 63.1 mmol), was added pyrrolidin-2-one (6.4 g, 75.2 mmol) and Cs₂CO₃ (30.8 g, 94.5 mmol). The resulting reaction mixture was argon degassed for 15 minutes. Then, to the degassed reaction mixture was added Pd(OAc)₂ (0.715 g, 3.2 mmol) and Xanthophos (3.6 g, 6.2 mmol) under argon, and the reaction mixture was heated to 100° C. in a sealed tube for 12 hours. The reaction mixture was evaporated to get crude material, which was purified using isolera column chromatography to afford 1-(3-nitropyridin-2-yl)pyrrolidin-2-one (7.5 g, 36.2 mmol, 57.4% yield) as a white solid, LCMS (ES⁺, m/z): 208.1 (M+1).

In step 2, to an ethanolic solution (100 ml) of 1-(3-nitropyridin-2-yl)pyrrolidin-2-one (10.9 g, 52.6 mmol) was added dry Pd/C (1.1 g). The resulting reaction mixture was kept stirring at RT under hydrogen atmosphere for 12 hours. TLC and LCMS of the reaction indicated complete consumption of starting material. The reaction mixture was diluted with ethanol and filtered through a bed of Celite. The filtered reaction mixture was evaporated to get crude material, which was purified using isolera column chromatography to afford 1-(3-aminopyridin-2-yl)pyrrolidin-2-one (7.3 g, 41.2 mmol, 78.0% yield) as a black-colored solid, LCMS (ES+, m/z): 178.1 (M+1).

In step 3, to a dimethyl formamide (70 ml) solution of 1-(3-aminopyridin-2-yl)pyrrolidin-2-one (7.3 g, 41.2 mmol) and 2,4,5-trichloropyrimidine (8.9 g, 48.5 mmol) in a sealed tube was added DIPEA (21.0 ml, 120.6 mmol). The resulting reaction mixture was heated to 80° C. for 12 hours. TLC and LCMS of the reaction indicated complete consumption of starting material. The reaction mixture was quenched with ice-cold water then extracted by ethyl acetate (2×50 ml). The combined organic layers were dried over Na₂SO₄, and evaporated to get crude material, which was purified using isolera column chromatography to afford 1-(3-((2,5-dichloropyrimidin-4-yl)amino)pyridin-2-yl)pyrrolidin-2-one (10.9 g, 33.6 mmol, 82.0% yield) as a brown-colored solid, LCMS (ES+, m/z): 324.0 (M+1).

In step 4, to a dimethyl formamide solution (50 ml) of 1-chloro-2-fluoro-4-methoxy-5-nitrobenzene (5.0 g, 24.4 mmol) was added 1-methylpiperazine (2.7 g, 26.9 mmol) and K₂CO₃ (4.3 g, 31.2 mmol) in a round-bottomed flask. The resulting reaction mixture was heated to 80° C. for 16 hours. TLC and LCMS of the reaction indicated complete consumption of starting material. The reaction mixture was quenched with ice-cold water, and the solid was filtered through a Buchner funnel to get pure 1-(2-chloro-5-methoxy-4-nitrophenyl)-4-methylpiperazine (6.6 g, 23.1 mmol, 95.0% yield) as a yellow-colored solid, LCMS (ES+, m/z): 286.1 (M+1).

In step 5, to an ethanolic solution (60 ml) of 1-(2-chloro-5-methoxy-4-nitrophenyl)-4-methylpiperazine (6.6 g, 23.1 mmol) was added Fe powder (6.3 g, 112.8 mmol), NH₄Cl (6.1 g, 114.1 mmol) and water (12.0 ml). The resulting reaction mixture was heated to 80° C. for 6 hours. TLC and LCMS of the reaction indicated complete consumption of starting material. The reaction mixture was evaporated to get crude material, which was purified using isolera column chromatography to afford 5-chloro-2-methoxy-4-(4-methylpiperazin-1-yl) aniline (5.2 g, 20.3 mmol, 88.0% yield) as a violet colour solid, LCMS (ES⁺, m/z): 256.1 (M+1).

In step 6, to a t-butanol solution (10.0 ml) of 1-(3-((2,5-dichloropyrimidin-4-yl)amino)pyridin-2-yl)pyrrolidin-2-one (1.0 g, 3.08 mmol) and 5-chloro-2-methoxy-4-(4-methylpiperazin-1-yl)aniline (0.787 g, 3.08 mmol) was added 1 ml TFA in a sealed tube, which was heated to 90° C. for 14 hours. After confirming the completion of reaction by TLC and LCMS, the reaction mixture was evaporated to get crude material. Then, the crude product was purified using PREP HPLC to afford 1-(3-((5-chloro-2-((5-chloro-2-methoxy-4-(4-methylpiperazin-1-yl)phenyl)amino)pyrimidin-4-yl)amino)pyridin-2-yl)pyrrolidin-2-one (0.7 g, 1.29 mmol, 42.0% yield) as a light brown-colored solid, ¹H-NMR (400 MHz, DMSO-d6): δ 8.86 (s, 1H), 8.31-8.29 (m, 1H), 8.24 (d, J=8.00 Hz, 1H), 8.17 (s, 1H), 7.91 (s, 1H), 7.75 (s, 1H), 7.40-7.37 (m, 1H), 6.76 (s, 1H), 4.02 (t, J=6.80 Hz, 2H), 3.82 (s, 3H), 2.96 (brs, 4H), 2.60 (t, J=6.80 Hz 2H), 2.24 (s, 3H), 2.13-2.1 (m, 3H); LCMS (ES⁺, m/z): 544.2 (M+1).

### Example 6. ApcMin Mouse Efficacy Model

TNK1 upregulation is reported to play a role in the pathogenic process of intestinal barrier disruption in a transgenic mice model of increased TNK1 expression/activation (PMID: 30320600). Therefore, the efficacy of the compound of structural formula I was evaluated in germline mutant mouse model of intestinal neoplasia and cachexia, Apc^{Min+/-} model, which is also known to induce gut barrier dysfunction (PMID: 21914473). Apc^{Min+/-} mice are reported to start the development of tumors at approximately 4 weeks of age. Twenty female Apc^{Min+/-} mice (JAX Stock No: 002020) were enrolled in the study at the age of 10 weeks. Randomization was done based on the body weight, and animals were divided into four treatment groups (5 mice/group). The treatment was initiated at day 1 after randomization and mice were administered vehicle (2% Tween-80, 10% ethanol, 30% PEG400 in water (v/v)), 25 or 50 mg/kg compound of structural formula I or 120 mg/kg celecoxib once daily via oral gavage. Celecoxib is a COX-2 inhibitor, and was used as a positive control because it has been reported to prevent and regress adenomas in APC model (PMID: 11016626). Mice were sacrificed 2 hours after the last dose of total 10 weeks of dosing (71 days), at the age of 20 weeks. After dosing started, the animals were checked daily for morbidity and mortality.

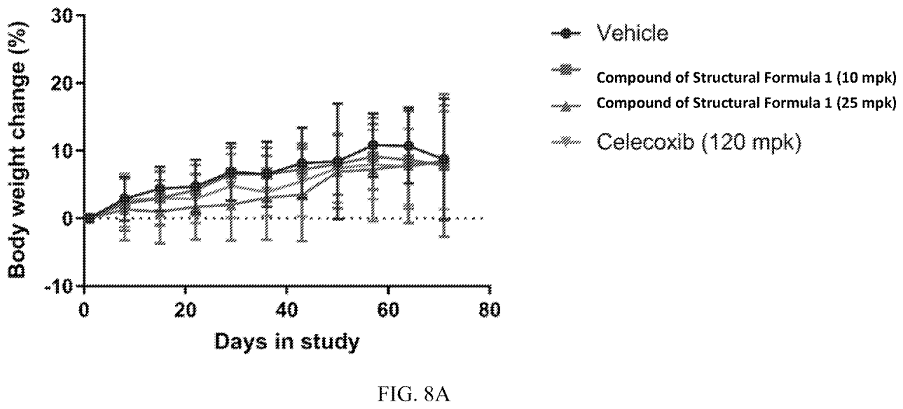

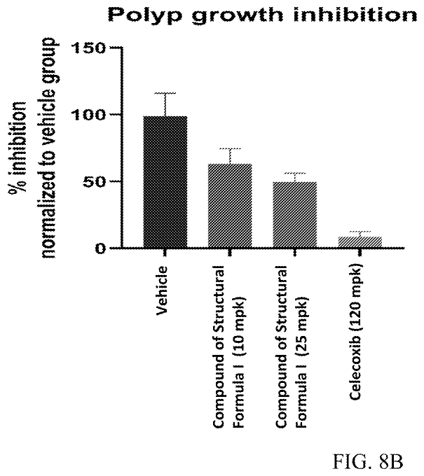

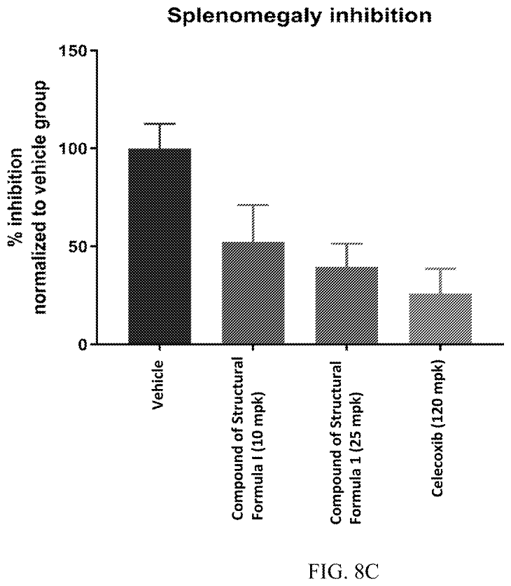

FIG. 8A shows that no significant differences were observed in body weight changes with treatments. FIG. 8B shows that a significant decrease in number of polyps was observed in the intestine of mice treated with compound of structural formula I (approximately 49% at 25 mg/kg) and celecoxib (approximately 90%) compared to mice administered vehicle.

Treatment groups also showed a significant reduction in spleen size compared to vehicle-treated group. FIG. 8C shows that 10 mg/kg compound of structural formula I reduced spleen sizes by approximately 52%, 25 mg/kg compound of structural formula I reduced spleen sizes by approximately 40% and 120 mg/kg celecoxib reduced spleen sizes by approximately 26%. The mean spleen weight of mice administered vehicle was 484 mg, the mean spleen weight of mice treated with 10 mpk compound of structural formula I was 253 mg, the mean spleen weight of mice treated with 25 mpk compound of structural formula I was 191 mg and the mean spleen weight of mice treated with 120 mpk celecoxib was 125 mg.

### Example 7. 3D Viability Assay

In cancer research, natural cell characteristics and architectures can often be more closely mimicked by 3D cell models than monolayer cultures. The comparative efficacy of compound of structural formula I was explored in 2D and 3D culture systems using the soft agar colony formation assay and PDXO cultures. In each case, viability of the culture was assessed after treatment with compound of structural formula I.

Soft Agar Colony Formation Assay. Soft agar colony formation assay was performed in 10 cell lines from a variety of tumor types ranging from lung cancers to colorectal and prostate cancers (Pro Quinase Reaction Biology, Project Number: 17262). A complete list of tumor types used in the assay can be found in Table 7.1. Table 7.1 shows that increased sensitivity to treatment with compound of structural formula I was observed in 3D colonies compared to 2D counterparts in most of the cell lines tested. The most prominent increase in sensitivity was observed in DU145, HT29 and A549 cells, where shifts in IC₅₀s of approximately 19-fold, approximately 32-fold and approximately 40-fold, respectively, were observed.

TABLE 7.1
IC₅₀ Values of Compound of Structural Formula
I in Soft Agar Colony Formation Assay

| Cell Line | Tissue Origin | 2D IC₅₀ (μM) | 3D IC₅₀ (μM) |
|---|---|---|---|
| DU145 | Prostate | 2.7 | 0.14 |
| HT29 | Colorectal | 7.68 | 0.24 |
| A549 | Lung | >10 | 0.25 |
| HCT116 | Colorectal | 3.36 | 0.75 |
| PC3 | Prostate | >10 | 0.87 |
| Lovo | Colorectal | 3.58 | 2.90 |
| SW948 | Colorectal | | 0.73 |
| C33A | Cervical | | 0.90 |
| A2058 | Melanoma | | 0.98 |
| T84 | Colorectal | | 1.5 |

Viability Assay in Patient-Derived Xenograft Organoid Cell Lines (PDXO). To mimic more closely cancer tissue-like architecture, PDX organoids derived from patient-derived xenograft tissue were utilized. Viability assay was performed in 10 PDXO models, and the IC₅₀ values were assessed after 5 days of treatment with compound of structural formula I using Cell Titre Glo. Table 7.2 shows that the compound of structural formula I had an IC₅₀ value<10 μM against all of the organoids tested, except CR6863B. The most responsive organoids were BL5001B, LU6800B, LU11873B and ES6470B, where compound of structural formula I exhibited IC₅₀ values of 0.40, 0.40, 0.44 and 0.51 μM respectively.

TABLE 7.2
IC₅₀ Values of Compound of Structural
Formula I in PDXO Viability Assay

| Organoid line | Tissue Origin | IC₅₀ (μM) |
|---|---|---|
| BL5001B | Bladder | 0.40 |
| CR1489B | Colorectal | 2.18 |
| CR5088B | Colorectal | 3.25 |
| CR6863B | Colorectal | 10.13 |
| GA6891B | Gastric | 1.24 |
| LU0743B | Lung | 2.43 |
| LU6800B | Lung | 0.40 |
| LU11693B | Lung | 2.12 |
| LU11873B | Lung | 0.44 |
| ES6470B | Esophageal | 0.51 |

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A solid form of a mesylate salt of a compound of the following structural formula: 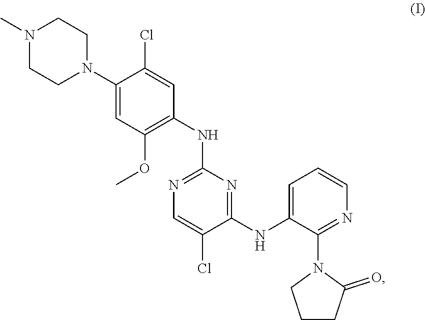 [SMILES: COc1cc(N2CCN(C)CC2)c(Cl)cc1Nc1ncc(Cl)c(Nc2cccnc2N2CCCC2=O)n1]
or a hydrate thereof.

2. A crystalline form of a mesylate salt of a compound of the following structural formula: 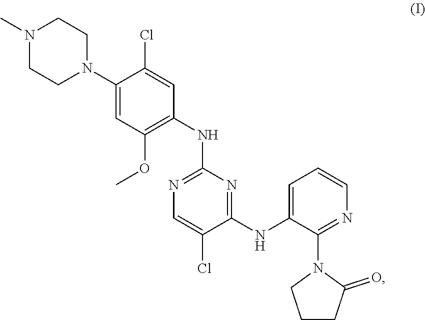 [SMILES: COc1cc(N2CCN(C)CC2)c(Cl)cc1Nc1ncc(Cl)c(Nc2cccnc2N2CCCC2=O)n1]
or a hydrate thereof.

3. The form of claim 2, comprising Type A.

4. The form of claim 2, consisting of Type A.

5. The form of any one of claims 1-4, wherein the form is substantially pure.

6. The form of any one of claims 1-5, characterized by an x-ray powder diffraction pattern comprising at least three peaks at 2-theta angles selected from the group consisting of 7.5±0.2°, 8.8±0.2°, 18.7±0.2°, 20.2±0.2° and 25.2±0.2°.

7. The form of claim 6, characterized by an x-ray powder diffraction pattern comprising at least four peaks at 2-theta angles selected from the group consisting of 7.5±0.2°, 8.8±0.2°, 18.7±0.2°, 20.2±0.2° and 25.2±0.2°.

8. The form of claim 7, characterized by an x-ray powder diffraction pattern comprising peaks at the following 2-theta angles: 7.5±0.2°, 8.8±0.2°, 18.7±0.2° and 20.2±0.2°.

9. The form of any one of claims 6-8, characterized by an x-ray powder diffraction pattern further comprising a peak at the following 2-theta angle: 16.9±0.2°.

10. The form of any one of claims 6-9, characterized by an x-ray powder diffraction pattern further comprising a peak at the following 2-theta angle: 12.4±0.2°.

11. The form of any one of claims 6-10, characterized by an x-ray powder diffraction pattern further comprising a peak at the following 2-theta angle: 20.6±0.2°.

12. The form of any one of claims 6-11, characterized by an x-ray powder diffraction pattern further comprising a peak at the following 2-theta angle: 15.2±0.2°.

13. The form of any one of claims 1-12, having an x-ray powder diffraction pattern substantially in accordance with that depicted in FIG. 1.

14. The form of claim 13, wherein the x-ray powder diffraction pattern is substantially in accordance with that depicted in FIG. 1 after storage for six months at about 25° C. and about 60% relative humidity in a closed container.

15. The form of any one of claims 6-14, wherein the x-ray powder diffraction pattern is as measured by x-ray powder diffraction using an x-ray wavelength of 1.5406 Å.

16. The form of any one of claims 1-15, characterized by a differential scanning calorimetry thermogram comprising an endothermic peak at 266° C.

17. The form of any one of claims 1-16, characterized by a differential scanning calorimetry thermogram comprising a thermal signal at 67° C.

18. The form of any one of claims 1-17, characterized by a differential scanning calorimetry thermogram substantially in accordance with that depicted in FIG. 2.

19. The form of claim 16, 17 or 18, wherein the differential scanning calorimetry thermogram is as measured by differential scanning calorimetry over a range of 25° C. to 300° C. using a scanning rate of 10° C./minute.

20. The form of any one of claims 1-19, characterized by a melting temperature of 262° C.

21. The form of any one of claims 1-20, characterized by a thermogravimetric analysis thermal curve with about 1.4% weight loss over the range of from about 25° C. to about 100° C.

22. The form of any one of claims 1-21, characterized by a thermogravimetric analysis thermal curve substantially in accordance with that shown in FIG. 2.

23. The form of claim 21 or 22, wherein the thermogravimetric analysis thermal curve is as measured using a heating rate of 10° C./minute.

24. The form of any one of claims 1-23, wherein the mesylate salt is a mesylate salt of the compound of structural formula I.

25. A pharmaceutical composition comprising a form of any one of claims 1-24 and a pharmaceutically acceptable carrier.

26. A pharmaceutical combination comprising a form of any one of claims 1-24 and one or more additional therapeutic agents.

27. A pharmaceutical composition comprising:
a compound of the following structural formula: 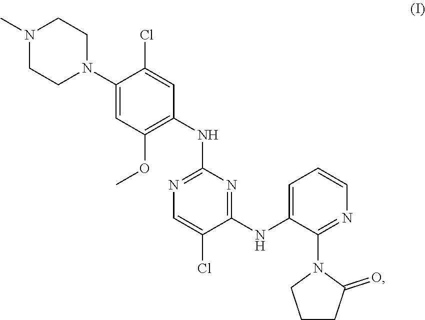 [SMILES: COc1cc(N2CCN(C)CC2)c(Cl)cc1Nc1ncc(Cl)c(Nc2cccnc2N2CCCC2=O)n1]
or a pharmaceutically acceptable salt thereof, or a hydrate of the foregoing; and
silicified microcrystalline cellulose; or
croscarmellose sodium; or
sodium stearyl fumarate.

28. The pharmaceutical composition of claim 27, comprising the compound of structural formula (I), or a pharmaceutically acceptable salt thereof, or a hydrate of the foregoing; silicified microcrystalline cellulose; croscarmellose sodium; and sodium stearyl fumarate.

29. The pharmaceutical composition of claim 27 or 28, comprising from about 5% to about 50% by weight of a compound of structural formula (I), or a pharmaceutically acceptable salt thereof, or a hydrate of the foregoing, based on the molecular weight of the compound of structural formula I as a free base.

30. The pharmaceutical composition of claim 29, comprising about 35% by weight of a compound of structural formula (I), or a pharmaceutically acceptable salt thereof, or a hydrate of the foregoing, based on the molecular weight of the compound of structural formula I as a free base.

31. The pharmaceutical composition of any one of claims 27-30, wherein the compound of structural formula (I) is in a form of any one of claims 1-24.

32. The pharmaceutical composition of any one of claims 27-31, comprising from about 25% to about 50% by weight silicified microcrystalline cellulose.

33. The pharmaceutical composition of claim 32, comprising about 30% by weight silicified microcrystalline cellulose.

34. The pharmaceutical composition of any one of claims 27-33, wherein the silicified microcrystalline cellulose has an average particle size by laser diffraction of about 125 μm.

35. The pharmaceutical composition of any one of claims 27-34, wherein the silicified microcrystalline cellulose has a bulk density of from about 0.25 to about 0.37 g/mL.

36. The pharmaceutical composition of any one of claims 27-35, comprising from about 1% to about 10% by weight croscarmellose sodium.

37. The pharmaceutical composition of claim 36, comprising about 5% by weight croscarmellose sodium.

38. The pharmaceutical composition of any one of claims 27-37, comprising from about 0.1% to about 5% by weight sodium stearyl fumarate.

39. The pharmaceutical composition of claim 38, comprising about 0.5% by weight sodium stearyl fumarate.

40. The pharmaceutical composition of any one of claims 27-39, further comprising mannitol, or a pharmaceutically acceptable salt thereof.

41. The pharmaceutical composition of claim 40, comprising from about 25% to about 50% by weight mannitol, or a pharmaceutically acceptable salt thereof.

42. The pharmaceutical composition of claim 41, comprising about 30% by weight mannitol, or a pharmaceutically acceptable salt thereof.

43. The pharmaceutical composition of any one of claims 40-42, wherein the mannitol, or a pharmaceutically acceptable salt thereof, is D-mannitol.

44. The pharmaceutical composition of any one of claims 40-43, wherein the mannitol, or a pharmaceutically acceptable salt thereof, has a d₁₀ of about 40 μm, a d₅₀ of about 130 μm and a d₉₀ of about 200 μm.

45. The pharmaceutical composition of any one of claims 25 and 27-44 or the pharmaceutical combination of claim 26, formulated for oral administration.

46. The pharmaceutical composition of any one of claims 25 and 27-45, containing less than 3% by weight water, as measured by Karl Fischer titration after four weeks at about 40° C. and about 75% relative humidity in a closed container.

47. The pharmaceutical composition of any one of claims 25 and 27-46, having a purity of at least 95%, as measured by high-performance liquid chromatography (HPLC) after four weeks at about 40° C. and about 75% relative humidity in a closed container.

48. The pharmaceutical composition of any one of claims 25 and 27-47, containing total impurities of less than 1%, as measured by HPLC after four weeks at about 40° C. and about 75% relative humidity in a closed container.

49. The pharmaceutical composition of claim 48, containing total impurities of less than 0.75%, as measured by HPLC after four weeks at about 40° C. and about 75% relative humidity in a closed container.

50. A unit dosage form comprising a pharmaceutical composition of any one of claims 25 and 27-49.

51. The unit dosage form of claim 50, comprising from about 5 mg to about 250 mg of the compound of structural formula I, or a pharmaceutically acceptable salt thereof, or hydrate of the foregoing, based on the molecular weight of the compound of structural formula I as a free base.

52. The unit dosage form of claim 50, comprising about 50 mg of the compound of structural formula I, or a pharmaceutically acceptable salt thereof, or hydrate of the foregoing, based on the molecular weight of the compound of structural formula I as a free base.

53. The unit dosage form of claim 50, comprising about 100 mg of the compound of structural formula I, or a pharmaceutically acceptable salt thereof, or hydrate of the foregoing, based on the molecular weight of the compound of structural formula I as a free base.

54. The unit dosage form of claim 50, comprising about 200 mg of the compound of structural formula I, or a pharmaceutically acceptable salt thereof, or hydrate of the foregoing, based on the molecular weight of the compound of structural formula I as a free base.

55. The unit dosage form of any one of claims 50-54, formulated for oral administration.

56. The unit dosage form of any one of claims 50-55, in the form of a capsule.

57. A method of treating a TNK1-mediated disease, disorder or condition in a subject in need thereof, comprising administering to the subject a therapeutically effective amount of a form of any one of claims 1-24, a pharmaceutical composition of any one of claims 25 and 27-49, a pharmaceutical combination of claim 26 or a unit dosage form of any one of claims 50-56.

58. The method of claim 57, wherein the TNK1-mediated disease, disorder or condition is a cancer, a gastrointestinal disorder, an inflammatory disorder, tissue injury, MODS, sepsis, an autoimmune disorder, a disease, disorder or condition of the microbiome or a disease, disorder or condition resulting from a trauma or intestinal injury.

59. A method of treating a cancer in a subject in need thereof, comprising administering to the subject a therapeutically effective amount of a form of any one of claims 1-24, a pharmaceutical composition of any one of claims 25 and 27-49, a pharmaceutical combination of claim 26 or a unit dosage form of any one of claims 50-56.

60. The method of claim 58 or 59, wherein the cancer comprises a solid tumor.

61. The method of any one of claims 58-60, wherein the cancer is pancreatic cancer.

62. The method of any one of claims 58-60, wherein the cancer is prostate cancer.

63. The method of claim 58 or 59, wherein the cancer is a hematologic cancer.

64. The method of claim 58, 59 or 63, wherein the cancer is an acute leukemia.

65. The method of claim 64, wherein the acute leukemia is acute myeloid leukemia or acute lymphocytic leukemia.

66. The method of claim 58, 59 or 63, wherein the cancer is a chronic leukemia.

67. The method of claim 66, wherein the chronic leukemia is chronic myeloid leukemia or chronic lymphocytic leukemia.

68. The method of claim 58, 59 or 63, wherein the cancer comprises a lymphoma.

69. The method of claim 58, 59, 63 or 68, wherein the cancer is Hodgkin's lymphoma.

70. The method of claim 58, 59, 63 or 68, wherein the cancer is non-Hodgkin's lymphoma.

71. The method of claim 58, 59 or 63, wherein the cancer is multiple myeloma.

72. The method of any one of claims 58-71, wherein the cancer is associated with a TNK1 mutation.

73. The method of claim 72, wherein the cancer is Hodgkin's lymphoma.

74. The method of claim 72, wherein the cancer is a colorectal cancer.

75. The method of claim 72, wherein the cancer is a lung cancer.

76. The method of claim 75, wherein the lung cancer is non-small cell lung cancer.

77. A method of treating a TNK1-mediated disease, disorder or condition in a subject carrying a TNK1 mutation, comprising:
determining whether the subject carries a TNK1 mutation; and
administering to the subject a therapeutically effective amount of a form of any one of claims 1-24, a pharmaceutical composition of any one of claims 25 and 27-49, a pharmaceutical combination of claim 26 or a unit dosage form of any one of claims 50-56 if it is determined that the subject carries the TNK1 mutation.

78. The method of any one of claims 72-77, wherein the TNK1 mutation is a C-terminal truncating mutation.

79. The method of claim 77 or 78, wherein the TNK1-mediated disease, disorder or condition is a cancer selected from colorectal cancer, Hodgkin's lymphoma or lung cancer.

80. A method of treating an inflammatory disorder or reducing inflammation in a subject in need thereof, comprising administering to the subject a therapeutically effective amount of a compound of the following structural formula: 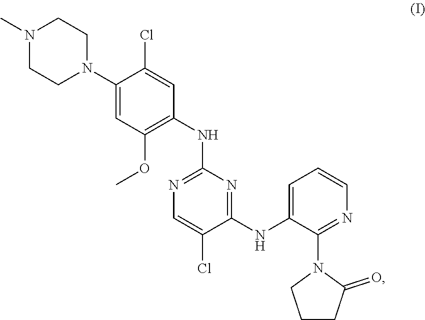 [SMILES: COc1cc(N2CCN(C)CC2)c(Cl)cc1Nc1ncc(Cl)c(Nc2cccnc2N2CCCC2=O)n1]
or a pharmaceutically acceptable salt thereof, or a hydrate of the foregoing, a form of any one of claims 1-24, a pharmaceutical composition of any one of claims 25 and 27-49, a pharmaceutical combination of claim 26 or a unit dosage form of any one of claims 50-56.

81. A method of treating tissue injury in a subject in need thereof, comprising administering to the subject a therapeutically effective amount of a compound of the following structural formula: 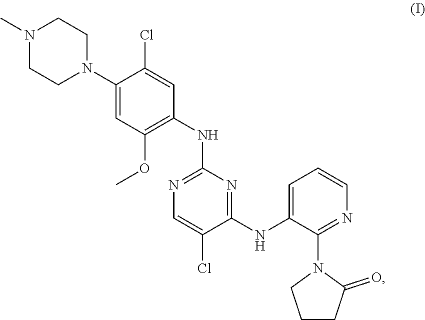 [SMILES: COc1cc(N2CCN(C)CC2)c(Cl)cc1Nc1ncc(Cl)c(Nc2cccnc2N2CCCC2=O)n1]
or a pharmaceutically acceptable salt thereof, or a hydrate of the foregoing, a form of any one of claims 1-24, a pharmaceutical composition of any one of claims 25 and 27-49, a pharmaceutical combination of claim 26 or a unit dosage form of any one of claims 50-56.

82. A method of improving intestinal barrier function in a subject in need thereof, comprising administering to the subject a therapeutically effective amount of a form of any one of claims 1-24, a pharmaceutical composition of any one of claims 25 and 27-49, a pharmaceutical combination of claim 26 or a unit dosage form of any one of claims 50-56.

83. A method of treating a disease, disorder or condition in a subject that would benefit from improved intestinal barrier function, comprising administering to the subject a therapeutically effective amount of a form of any one of claims 1-24, a pharmaceutical composition of any one of claims 25 and 27-49, a pharmaceutical combination of claim 26 or a unit dosage form of any one of claims 50-56.

84. The method of claim 83, wherein the disease, disorder or condition is a cancer, a gastrointestinal disorder, an inflammatory disorder, tissue injury, multi-organ dysfunction syndrome (MODS), sepsis, an autoimmune disorder, a disease, disorder or condition of the microbiome, or a disease, disorder or condition resulting from a trauma or intestinal injury.

85. The method of claim 58 or 84, wherein the gastrointestinal disorder is multiple intestinal neoplasia, ischemia/reperfusion injury, colitis, infectious diarrhea, celiac disease or inflammatory bowel disease (IBD).

86. The method of claim 58, 80, 84 or 85, wherein the inflammatory disorder is chronic obstructive pulmonary disease (COPD), allergies, cardiovascular disease, hepatitis, asthma, systemic inflammatory response syndrome (SIRS), multiple sclerosis, Goodpasture syndrome, psoriasis, ankylosing spondylitis, antiphospholipid antibody syndrome, gout, arthritis, myositis, scleroderma, Sjogren's syndrome, systemic lupus erythematosus or vasculitis.

87. The method of any one of claims 58, 81 and 84-86, wherein the tissue injury is induced by trauma, hemorrhagic shock or physical, chemical or polytrauma.

88. The method of any one of claims 58 and 84-87, wherein the autoimmune disorder is fibrosis, rheumatoid arthritis, multiple sclerosis, systemic lupus erythematosus, type 1 diabetes mellitus, Guillain-Barre syndrome, chronic inflammatory demyelinating polyneuropathy, Graves' disease, Hashimoto's thyroiditis, myasthenia gravis, IBD, polymyositis, dermatomyositis, inflammatory myositis, ankylosing spondolytis, ulcerative colitis, psoriasis, vasculitis, Sjogren's disease or transplant rejection.

89. A method of treating splenomegaly in a subject in need thereof, comprising administering to the subject a therapeutically effective amount of a form of any one of claims 1-24, a pharmaceutical composition of any one of claims 25 and 27-49, a pharmaceutical combination of claim 26 or a unit dosage form of any one of claims 50-56.

90. The method of any one of claims 57-89, further comprising administering to the subject one or more additional therapeutic agents.

91. The method of claim 90, comprising administering to the subject one or more standard of care agents.

92. The method of claim 90 or 91, comprising administering to the subject a proteasome inhibitor.

93. The method of claim 92, wherein the proteasome inhibitor is selected from bortezomib, N-5-benzyloxycarbonyl-Ile-Glu(O-tert-butyl)-Ala-leucinal, carfilzomib, ixazomib, marizomib (NPI-0052), delanzomib (CEP-18770), or O-methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912), or a pharmaceutically acceptable salt thereof.

94. The method of claim 93, wherein the proteasome inhibitor is bortezomib, or a pharmaceutically acceptable salt thereof.

95. The method of any one of claims 90-94, wherein the disease, disorder or condition is multiple myeloma.

96. The method of any one of claims 90-95, comprising administering to the subject an immune checkpoint inhibitor.

97. The method of claim 96, wherein the immune checkpoint inhibitor is a PD-1 inhibitor, PD-L1 inhibitor or CTLA-4 inhibitor.

98. A method of mediating apoptosis in a cell, comprising contacting the cell with a compound of the following structural formula: 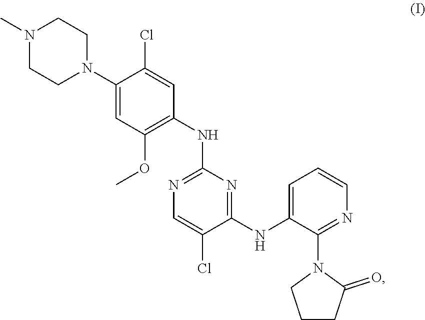 [SMILES: COc1cc(N2CCN(C)CC2)c(Cl)cc1Nc1ncc(Cl)c(Nc2cccnc2N2CCCC2=O)n1]
or a pharmaceutically acceptable salt thereof, or a hydrate of the foregoing, a form of any one of claims 1-24, a pharmaceutical composition of any one of claims 25 and 27-49, a pharmaceutical combination of claim 26 or a unit dosage form of any one of claims 50-56.

99. A method of reducing inflammation in a cell, comprising contacting the cell with a compound of the following structural formula: 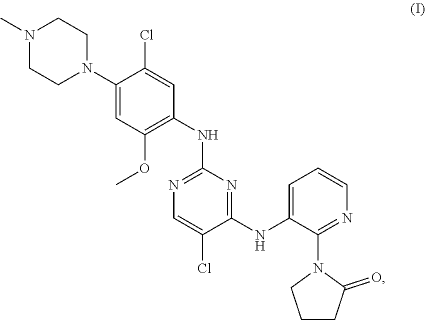 [SMILES: COc1cc(N2CCN(C)CC2)c(Cl)cc1Nc1ncc(Cl)c(Nc2cccnc2N2CCCC2=O)n1]
or a pharmaceutically acceptable salt thereof, or a hydrate of the foregoing, a form of any one of claims 1-24, a pharmaceutical composition of any one of claims 25 and 27-49, a pharmaceutical combination of claim 26 or a unit dosage form of any one of claims 50-56.

100. A method of inhibiting TNK1 activity in a cell, comprising contacting the cell with a form of any one of claims 1-24, a pharmaceutical composition of any one of claims 25 and 27-49, a pharmaceutical combination of claim 26 or a unit dosage form of any one of claims 50-56.

101. The method of claim 98, 99 or 100, wherein the cell is in a human.

102. A method of inhibiting TNK1 activity in a subject in need thereof, comprising administering to the subject a therapeutically effective amount of a form of any one of claims 1-24, a pharmaceutical composition of any one of claims 25 and 27-49, a pharmaceutical combination of claim 26 or a unit dosage form of any one of claims 50-56.

103. The method of any one of claims 100-102, wherein the TNK1 carries a mutation.

104. The method of claim 103, wherein the mutation is a truncating mutation.

105. The method of any one of claims 57-97 and 102-104, comprising administering to the subject from about 62 mg to about 229 mg of the compound of structural formula I, or a pharmaceutically acceptable salt thereof, or a hydrate of the foregoing, based on the molecular weight of the compound of structural formula I as a free base.

106. A method of making a mesylate salt of a compound of the following structural formula: 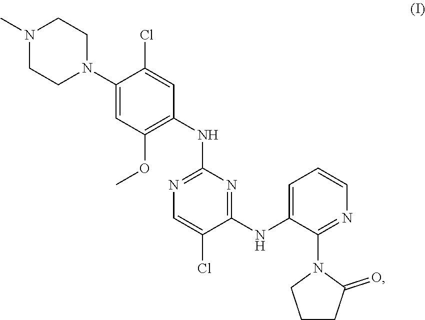 [SMILES: COc1cc(N2CCN(C)CC2)c(Cl)cc1Nc1ncc(Cl)c(Nc2cccnc2N2CCCC2=O)n1]
or a hydrate thereof, comprising contacting the compound of structural formula I with methanesulfonic acid in a solvent, thereby making the mesylate salt of a compound of structural formula I, or a hydrate thereof.

107. The method of claim 106, comprising forming a mixture of the compound of structural formula I in the solvent, and contacting the mixture with methanesulfonic acid, thereby making the mesylate salt of a compound of structural formula I, or a hydrate thereof.

108. The method of claim 107, wherein the mixture is a solution.

109. The method of claim 107, wherein the mixture is a suspension.

110. The method of any one of claims 106-109, wherein the solvent is an organic solvent, or an aqueous mixture thereof.

111. The method of claim 110, wherein the solvent is tetrahydrofuran, a mixture of acetonitrile and water, acetone or ethyl acetate.

112. The method of any one of claims 106-111, further comprising precipitating the mesylate salt of a compound of structural formula I, or a hydrate thereof.

113. The method of claim 112, further comprising isolating the precipitated mesylate salt of a compound of structural formula I, or a hydrate thereof.
